(19) United States
(12) Patent Application Publication
(10) Pub. No.: US 2026/0200923 A1
(43) Pub. Date: Jul. 16, 2026

(54) HETEROCYCLIC COMPOUNDS TARGETING THE NUDT5 PROTEIN

(71) Applicant: Oxford University Innovation Limited, Oxford (GB)

(72) Inventors: Kilian HUBER, Oxford (Oxfordshire) (GB); Anne-Sophie MARQUES, Oxford (Oxfordshire) (GB); Stefan KUBICEK, Vienna (AT); George LIN, Vienna (AT)

(21) Appl. No.: 19/133,942

(22) PCT Filed: Dec. 14, 2023
(86) PCT No.: PCT/GB2023/053237

§ 371 Date: May 29, 2025

(30) Foreign Application Priority Data
Dec. 14, 2022 (EP) 22306873.5
Feb. 21, 2023 (GB) 2302439.1

(51) Int. Cl. C07D 473/08; A61K 31/522

(52) U.S. Cl. CPC C07D 473/08; A61K 31/522

(57) ABSTRACT

The invention concerns compounds which are useful in targeting and degrading the NUDT5 protein. The invention also concerns the compounds of the invention for use in methods of treatment, particularly for use in treatment of cancer, inflammatory disorders and folate-related disorders, as well as to reduce the side-effects and/or toxicity of a second drug. The invention further concerns screening methods.

### FIELD OF THE INVENTION

The invention provides a compound of formula (I), formula (IA), or formula (IB), which is useful in targeting and degrading the NUDT5 protein. The invention also provides the compounds of the invention for use in methods of treatment, particularly for use in treatment of cancer and inflammatory disorders, as well as to reduce the side-effects and/or toxicity of a second drug. The invention further provides screening methods.

### BACKGROUND TO THE INVENTION

Folate metabolism is essential for providing one-carbon units to the biosynthesis of numerous metabolites, including purines, thymidylate, and methionine (1). Consequently, impairment of folate metabolism via gene mutations or dietary folate deficiency causes varied pathologies including developmental defects and an increased risk of cancer (2-6). Therapeutically, the dependence of proliferating cells on this pathway is exploited by the use of antifolates in cancer therapy (7, 8).

The folate pathway is compartmentalized between cytoplasm and mitochondria, with additional roles of selected enzymes in the nucleus (9, 10). The normal direction of the folate catalytic cycle of mitochondrial formate production and cytosolic formate utilization can be reversed following mutation of key folate enzymes(11). Under these conditions, as well as pharmacological inhibition with cancer drugs like methotrexate, destabilization of the folate scaffold might further contribute to the overall phenotype (12, 13). MTHFD1 (C-1 tetrahydrofolate synthase) is a tri-functional enzyme that catalyzes the cytoplasmic interconversion of 10-formyltetrahydrofolate (10-CHO-THF), methenyltetrahydrofolate (CH⁺-THF), and methylenetetrahydrofolate (CH₂-THF) by its formyltetrahydrofolate synthetase and methylenetetrahydrofolate dehydrogenase/methenyltetrahydrofolate cyclohydrolase domains (FIG. 1A). While CH₂-THF provides one-carbon units for the synthesis of methionine and thymidylate, 10-CHO-THF delivers two of the carbons to the purine scaffold generated in the de novo synthesis pathway. This process might be enhanced by direct interaction of MTHFD1 with the purinosome (14). Knock-out of MTHFD1 in yeast and human cells results in purine auxotrophy, presumably by preventing purine de novo synthesis (15-20). In contrast, in cells from MTHFD1 deficient patients purine de novo synthesis was unaffected, while thymidylate and methionine biosynthesis were impaired due to point mutations in the cyclohydrolase domain of the enzyme (21). To date, no comprehensive studies of the metabolic, pharmacologic, and genetic dependencies caused by loss of the distinct MTHFD1 enzymatic activities are available. Performing such analysis, the present inventors have now shown a key role of MTHFD1 for switching between adenosine requirement and toxicity, and find that the antiproliferative effect of adenosine and other purine analogs in this context is dependent on a previously unidentified scaffolding function of NUDT5 (also referred to as NUDIX5), revealing this NUDIX family member as a key regulator of de novo purine synthesis.

The inventors have recognised that, in light of this mechanism, reducing the activity of protein NUDT5 would have a number of therapeutic benefits due to its knock-on effect on the crucial MTHFD1 pathway. The inventors and other workers in this field have tested the efficacy of inhibitors of NUDT5, but found that these inhibitors do not yield a satisfactory result in terms of observed disruption of the MTHFD1 pathway. However, the inventors have now developed novel degrader molecules which degrade rather than inhibit the NUDT5 pathway. These have been found to be associated with exceptionally high activity and corresponding therapeutic benefits.

### SUMMARY OF THE INVENTION

Accordingly, the invention provides a compound of formula (I):

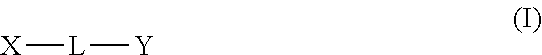

or a pharmaceutically acceptable salt thereof,
wherein X is a moiety capable of binding to NUDT5;
L is a linker comprising at least one heterocyclic moiety, said heterocyclic moiety being a bivalent, saturated, nitrogen-containing heterocyclic moiety; and
Y is a moiety capable of binding to E3 ligase.

Without wishing to be bound by theory, it is speculated that the particular heterocycle-containing linkers L provide an excellent balance of properties enabling the degraders to act efficiently. The linkers may be sufficiently rigid to hold the X and Y groups together in an advantageous conformation without leading to steric stress, while also providing excellent solubility characteristics.

In addition to the above-described linkers L, the inventors have also identified a second group of linkers which provide excellent degrading activity when used to couple X and Y groups. These are believed to provide an alternative route to the excellent balance of properties enabling the degraders to act efficiently. Accordingly, the invention also provides a compound of formula (IA):

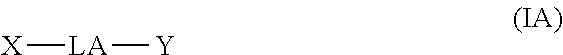

or a pharmaceutically acceptable salt thereof,
wherein X is a moiety capable of binding to NUDT5;
LA is a linker of formula:

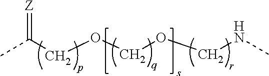

wherein
Z is O or S;
p, q, r and s are each integers independently selected from 1, 2, 3 or 4, each q being independently selected from 1, 2, 3 or 4 where s>1; and
Y is a moiety capable of binding to E3 ligase.

An E3 ligase is a species which promote the ubiquitination of proteins, and the consequent destruction of those proteins by the proteasome. Thus, bringing an E3 ligase into close contact with NUDT5 leads to the destruction of the NUDT5 protein. However, E3 ligases are not the only species which are capable of flagging a protein for destruction by the proteasome (or recruiting the protein into any other destruction pathway). The skilled person is aware of numerous pathways by which a protein may be destroyed by existing machinery within living cells, and of small molecules which are known to bind to chemical species active within those pathways. Any such protein-destroying pathway may in principle be utilised to destroy the NUDT5 protein by a degrader according to the present invention.

Thus, also provided herein is a compound of formula (IB):

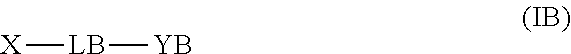

or a pharmaceutically acceptable salt thereof,
wherein X is a moiety capable of binding to NUDT5;
LB is a linker; and
YB is a moiety capable of recruiting cellular destruction machinery, except that YB is not a moiety capable of binding to VHL or CRBN.

It is particularly preferred that X is the following group:

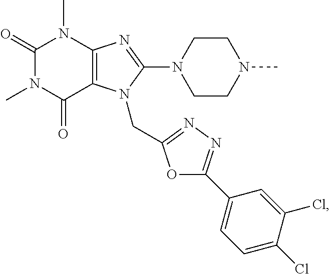

where a dotted line indicates a covalent bond to L.

Thus, the invention also provides a compound of formula (I′):

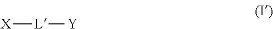

or a pharmaceutically acceptable salt thereof, wherein
X is a group of formula

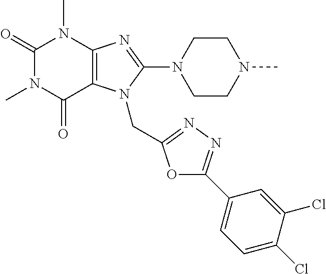

L′ is a linker (for instance, selected from a linker L as described herein, C₁-C₆ alkylene; —NH—, —O—, —C(═O)—, and any combination thereof); and
Y is a moiety capable of binding to E3 ligase.

The invention further provides a pharmaceutical composition comprising a compound of the invention and a pharmaceutically excipient or carrier.

The present invention also provides a compound of the invention for use in the treatment of the human or animal body.

The invention also provides a compound of the invention for use in a method of treating an inflammatory disorder, autoimmune disorder, cancer or metabolic disorder in a subject.

The invention further provides a compound of the invention for use in a method of reducing the side-effects or toxicity of a second drug, wherein the subject is also being treated with said second drug.

The invention also provides a compound of the invention and methotrexate for simultaneous, separate, or sequential administration to treat cancer or arthritis.

The invention further provides a compound of the invention and 6-thioguanine for simultaneous, separate, or sequential administration to treat cancer.

The invention also provides a compound of the invention for use in a method of treatment of a subjecting by inhibiting NUDT14.

Also provided is a compound able to reduce the association of NUDT5 and phosphoribosyl pyrophosphate amidotransferase (PPAT) for use in a method of treatment.

Further provided is a compound able to reduce the association of NUDT5 and phosphoribosyl pyrophosphate amidotransferase (PPAT) for use in a method of treating an inflammatory disorder, autoimmune disorder, cancer or metabolic disorder in a subject.

Also provided is a method of screening for a therapeutic agent comprising determining the ability of a test agent to reduce the association between NUDT5 and PPAT.

### BRIEF DESCRIPTION OF THE FIGURES

FIG. 1. MTHFD1 enzymatic functions control a switch between adenosine dependency and toxicity. (A) Schematic overview of the enzymatic functions exerted by the MTHFD1 synthetase (S), cyclohydrolase (C) and dehydrogenase (D) activities, and corresponding folate intermediate metabolites. (B) Domain structure of MTHFD1 showing K56R and K386E mutations that ablate the enzymatic reactions of each respective domain. (C) Normalized cell growth of WT HAP1, MTHFD1^{KO}, MTHFD1^{386E}, and MTHFD1^{K56R} cells to adenosine supplementation. Depicted are cell numbers following 72 h growth in media containing dialyzed serum (DIA) or dialyzed serum supplemented with 50 μM adenosine (ADE), normalized to the respective clone grown in media containing undialyzed serum (FULL). n=3 biological replicates, Mean±s.d, *p<0.05. (D) Representative cell cycle plots for WT and MTHFD1 mutant HAP1 cells in conditions of adenosine supplementation. (E) Representative images of γH2AX and RPA2 staining of MTHFD1^{K56R} cells with adenosine supplementation. Scale bar is 10 μm. (F) Growth assays of fibroblasts derived from MTHFD1 deficiency patients in full, dialyzed and adenosine supplemented media conditions (n=3 biological replicates, Mean±s.d, *p<0.05).

FIG. 2. Genome-wide knockout screens identify modulators of MTHFD1-mediated adenosine responses. (A) Schematic overview of the genome-wide CRISPR screens. (B) Significantly depleted genes, colored by essentiality, in the genome-wide knock-out screen in MTHFD1^{KO} cells treated for two weeks with adenosine. (C) Cell number quantification of MTHFD1^{KO}, MTHFD1^{KO}PML^{KO}, MTHFD1^{KO}PARP8^{KO}, MTHFD1^{KO}ATF7^{KO} cells grown in FULL, DIA, and ADE media for 72 h. (n=3 biological replicates, mean±s.d, *p<0.05) (D) Representative western blots for phospho-AMPK and AMPK protein levels in WT, MTHFD1^{KO}, MTHFD1^{KO}PML^{KO}, and MTHFD1^{KO}PARP8^{KO} cells pretreated in FULL, DIA, ADE for 24 h. (E) Results of the genome-wide enrichment screen of MTHFD1^{K56R} cultivated for two weeks with, in that context, toxic concentrations of 50 μM adenosine. (F) Normalized cell number of the parental MTHFD1^{K56R} cell line and two clonal lines of MTHFD1^{K56R}NUDT5^{KO} in adenosine supplemented media (ADE). Cell numbers were normalized to the FULL media condition for each respective cell line (n>2 biological replicates, mean±s.d, *p<0.05). (G) Representative western blots for phospho-AMPK and AMPK protein levels in WT, NUDT5^{KO}, MTHFD1^{K56R}, and MTHFD1^{K56R}NUDT5^{KO} cells pretreated in FULL or ADE for 24 h.

FIG. 3. Knock-out or chemical degradation, but not enzymatic inhibition, of NUDT5 prevents adenosine-mediated toxicity. (A) Normalized cell counts of MTHFD1^{K56R} cells treated for 72 h with 50 μM adenosine and 10 μM NUDT5 inhibitor TH5427 alone and in combination. (B) Normalized cell counts of MTHFD1^{KO}NUDT5^{KO} double knock-out cells reconstituted with either wildtype NUDT5 or the catalytically inactive NUDT5(E 112Q) mutant cultivated in FULL and ADE media conditions for 72 h. (C) Chemical structure of the NUDT5 targeting PROTAC dNUDT5 and its inactive analog dNUDT5nc. (D) Representative Western blot of dose-dependent dNUDT5 effects on NUDT5 protein levels after 20 h treatment in HAP1 cells. (E) Time-dependence of NUDT5 degradation with 100 nM dNUDT5 in HAP1 cells. (F) Competition of NUDT5 degradation by inhibitors of NUDT5 (TH5427), Nedd8 activating enzyme (MLN4924) and the proteasome (MG132) in HEK293 cells. (G) Untargeted global proteomics of HAP1 cells treated with 100 nM dNUDT5 for 6 and 24 h. (H) Overlay of the crystal structures of NUDT5 bound to TH5427 and dNUDT5. (I) Dose dependent effects of dNUDT5 on the growth of WT and MTHFD1^{K56R} cells cultivated in media with and without adenosine supplementation for 72 h.

FIG. 4. NUDT5 binds to PPAT to repress de novo purine synthesis. (A) Quantitative proteomics analysis of proteins pulled-down using a TH5427 affinity probe in HAP1 cell lysates treated with control DMSO or an excess of 20 μM free TH5427 as competitor. (B) Western blot for NUDT5 and PPAT following pull-down using a TH5427 affinity probe in HAP1 cell lysates treated with control DMSO or an excess of 20 μM free TH5427 as competitor. (C) Immunoprecipitation of endogenous PPAT following treatment of HAP1 cells with 100 nM dNUDT5, dNUDT5nc and TH5427 for 20 h. (D) Immunoprecipitation of endogenous PPAT following treatment of HAP1 cells with 50 μM adenosine and 100 nM dNUDT5 alone and in combination for 24 h. (E) Schematic overview of the purine de novo synthesis and salvage pathways. (F) Ratio of de novo versus salvage derived IMP (M+4/M+2) and the ratio of de novo versus unlabelled IMP (M+2/M). n>2, ****,*p<0.05.

FIG. 5. Large-scale compound screen reveals that only adenine-containing compounds rescue the growth of MTHFD1^{KO} cells. (A) Schematic of the two-stage compound screen workflow. (B) Chemical structures of the validated hit compounds. (C) Normalized cell numbers of MTHFD1^{KO} cells grown in dialyzed media supplemented with the validated hits from the screen and various folate intermediates (n=2 biological replicates, mean±s.d, *p<0.05). (D) Normalized cell numbers of MTHFD1^{KO} cells grown in dialyzed media supplemented with various nucleosides and nucleotides. (n=2 biological replicates, mean±s.d, *p<0.05). (E) MTHFD1 reintroduction into MTHFD1^{KO} cells abolishes effects of dialyzed serum and adenosine supplementation.

FIG. 6. Quantification of cell cycle and DNA damage effects of adenosine supplementation. (A) Quantification of the percentage of the cells in specific cell cycle states. Cells were pretreated in FULL, DIA, or ADE for 24 h. (n=3 biological replicates, mean±s.d). (B) Quantification of the average number of γH2AX nuclear foci and the integrated intensity (size and brightness) of RPA2 foci of WT and K56R cells grown in FULL, DIA, or ADE media (n=3 biological replicates, mean±s.d, *p<0.05).

FIG. 7. Adenosine supplementation leads to DNA damage and cell cycle arrest in MTHFD1-deficient, patient-derived fibroblasts. (A) Schematic representation of MTHFD1 domain architecture as well as the patient-specific mutations. (B) Highest enriched GO terms comparing the transcriptome of patient cells in ADE versus DIA media. (C) Representative images and quantification of the γH2AX foci per nuclei of patient 2 cells pretreated in FULL, DIA, ADE media for 24 h. (n=3 biological replicates, Mean±s.d, *p<0.05). (D) Cell cycle analysis of patient 2 cells pretreated in FULL, DIA, ADE media conditions. (n=3 biological replicates, Mean±s.d, *p<0.05).

FIG. 8. Adenosine supplementation jumpstarts internal OXPHOS-based ATP production in MTHFD1^{KO} cells. (A) CellTiter-Glo (CTG) assay reveals a dose-dependent increase in ATP levels of MTHFD1^{KO} cells after a 90 min incubation with 50 M adenosine. (n=4 biological replicates, Mean±s.d, *p<0.05). (B) Quantification of western blot signals phospho-AMPK and AMPK protein levels in WT, MTHFD1^{KO}, MTHFD1^{KO}PML^{KO}, MTHFD1^{KO}PARP8^{KO} pretreated in FULL, DIA, ADE for 24 h. (n=3 biological replicates, Mean±s.d, *p<0.05). (C) Representative OCR and ECAR graphs of the Seahorse bioenergetics studies of MTHFD1^{KO}, MTHFD1^{KO}PML^{KO}, MTHFD1^{KO}PARP8^{KO} cells grown in FULL or ADE media. Scale bars are mean±s.d. (E) The normalized levels total ATP production, ATP production by glycolysis, and ATP production by OXPHOS in MTHFD1^{KO}, MTHFD1^{KO}PML^{KO}, MTHFD1^{KO}PARP8^{KO} cells pretreated with FULL or ADE media for 24 h. Results were normalized to the MTHFD1^{KO} FULL condition (n=2 biological replicates, Mean±s.d, *p<0.05, one-way ANOVA with Tukey's).

FIG. 9. Reversal of adenosine-mediated toxicity in MTHFD1 mutant cells by dNUDT5. (A) NanoBRET assay for target engagement of compounds to luciferase-tagged NUDT5 following 2 h treatment in dose-response. (B) Ternary complex formation assay for the NUDT5-CRBN interaction in using the nanoBRET protein-protein interaction format following compound treatment for 4 h. (C) Representative Western blot of dose-dependent dNUDT5 effects on NUDT5 protein levels after 20 h treatment in HEK293 cells. (D) Time-dependence of NUDT5 degradation with 100 nM dNUDT5 in HEK293 cells. (E) Western blot quantification for DC50 calculation of dNUDT5 in HEK293 and HAP1 cells. (F) Control experiments for NUDT5 degradation at 100 nM compound treatment for 6 h in HAP1 and HEK293 cells. (G) Normalized cell growth of MTHFD1^{K56R} cells treated with various concentrations of dNUDTnc. (n=2 biological replicates, Mean±s.d, *p<0.05). (H) Normalized cell growth of two patient-derived fibroblast lines, which harbor MTHFD1 mutations. (n=3 biological replicates, Mean±s.d, *p<0.05).

FIG. 10. Loss of NUDT5 leads to increased de novo purine synthesis production and decreased purine salvage. (A) Total levels of measured IMP, including labelled and unlabelled species. n>2 biological replicates, Mean±s.d, ****p<0.05. (B) Ratio of de novo versus salvage derived (M+4/M+2), the ratio of de novo versus unlabelled (M+2/M) and total ATP. n>2, Mean±s.d, ****, **, *p<0.05. (C) Ratio of de novo versus salvage derived (M+4/M+2), the ratio of de novo versus unlabelled (M+2/M) and total GTP. n>2, Mean±s.d, ****, **, *p<0.05.(D) Total levels of intracellular isotope labelled Adenosine. n>2, Mean±s.d, ****, **, *p<0.05.

FIG. 11. NUDT5 degradation prevents 6-thioguanine (6-TG) toxicity. (A) Dose-response curves for dNUDT5, dNUDT5nc and TH5427 in HAP1 and NB4 cells treated with 0.2 μg/ml 6-thioguanine or control DMSO for 72 h. (B) Quantification of relative luminescence of CellTiteGlo signal measuring ATP levels in HAP1 and NB4 cells treated with 0.2 μg/ml 6-thioguanine or control DMSO for 72 h.

FIG. 12. In vitro characterization of NUDT5 degraders. (A) Degradation efficiency of NUDT5 degraders in HEK293 cells after 20 h across five concentrations. Degradation was calculated from normalized Western Blot intensities from two biological replicates. (B) NanoBRET PPI assay of NL-NUDT5 and HT-CRBN in HEK293 cells after 4 h. (C) Representative Western Blot of NUDT5 degradation in HAP-1, A549 and MCF7 cells with 100 nM compounds after 20 h. Bar graph shows median of normalized NUDT5 levels of 4 biological replicates. (D) CRBN knock-out rescues degrader-mediated NUDT5 degradation in HEK293 cells after 20 h. (E) NUDT5 degraders rescue 6-TG induced toxicity in dose dependent fashion. (F) Cell viability is unaffected by degraders treatment in HEK293 and HAP-1 cells after 72 h measured with CTG assay.

### DETAILED DESCRIPTION OF THE INVENTION

### Definitions

As used in this specification and the appended claims, the singular forms “a”, “an”, and “the” include plural referents unless the content clearly dictates otherwise. Thus, for example, reference to “a fluorophore” includes two or more fluorophores; or reference to “a protein” includes one or more proteins.

Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to provide the disclosed products and perform the disclosed methods.

“Alkyl” as used herein refers to monovalent straight-chained and branched alkyl groups. Typically, the alkyl group is a straight-chained alkyl group. An alkyl group may have from 1 to 30 carbon atoms (i.e. is a C₁₋₃₀ alkyl group). Typically, an alkyl group is a C₁₋₂₀ alkyl group or a C₁₋₁₀ alkyl group. Preferred alkyl groups include C₁₋₆ alkyl groups, for example C₁₋₄ alkyl groups. Examples of alkyl groups include methyl and ethyl groups, and straight-chained or branched propyl, butyl and pentyl groups. Particular alkyl groups include methyl, ethyl, n-propyl, isopropyl, n-butyl, sec-butyl, isobutyl, and tert-butyl groups.

“Alkenyl” as used herein refers to a monovalent hydrocarbon moiety comprising one or more carbon-carbon double bonds. Typically an alkenyl group contains one carbon-carbon double bond. The hydrocarbon moiety may be a straight-chain or branched; typically, the hydrocarbon moiety is a straight chain. An alkenyl group may have from 2 to 30 carbon atoms (i.e. is a C₂₋₃₀ alkenyl group). Typically, an alkenyl group is a C₂₋₂₀ or a C₂₋₁₀ alkenyl group. Preferred alkenyl groups include C₂₋₆ alkenyl groups, for example C₂₋₄ alkenyl groups.

“Alkoxy” as used herein refers to a moiety of the formula —O-alkyl, where an alkyl group is as defined herein. Preferred alkoxy groups include —O—C₁₋₆ alkoxy groups, for example —O—C₁₋₄ alkoxy groups. Examples of alkoxy groups include methoxy and ethoxy groups.

“Alkylthio” as used herein refers to a moiety of the formula —S-alkyl, where an alkyl group is as defined herein. Preferred alkylthio groups include —S—C₁₋₆, for example —S—C₁₋₄ groups. Examples of alkylthio groups include methylthio and ethylthio groups.

“Alkylene” as used herein refers to a divalent saturated hydrocarbon moiety which may be straight-chained or branched. Typically, the alkylene group is a straight-chained alkylene group. An alkylene group typically has from 1 to 10 carbon atoms (i.e. is a C₁₋₁₀ alkylene group). However, preferred alkylene groups include C₁₋₆ alkylene groups, especially C₁₋₄ alkylene groups. Examples of alkylene groups include methylene (—CH₂—) and ethylene (—CH₂CH₂—) groups.

“Alkyleneoxy” as used herein refers to a moiety of the formula —O-alkylene-, where an alkylene group is as defined herein.

“Alkylenethio” as used herein refers to a moiety of the formula —S-alkylene-, where an alkylene group is as defined herein.

“Alkenylene” as used herein refers to a divalent hydrocarbon moiety comprising one or more carbon-carbon double bonds. Typically an alkenylene group contains one carbon-carbon double bond. The hydrocarbon moiety may be a straight-chain or branched; typically, the hydrocarbon moiety is a straight chain. An alkenylene group typically has from 2 to 10 carbon atoms (i.e. is a C₂₋₁₀ alkenylene group). However, preferred alkenylene groups include C₂₋₆ alkenylene groups, for example C₂₋₄ alkenylene groups.

The term “aminoalkyl” as used herein refers to a moiety of the formula —NH-alkyl or —N(alkyl)₂, wherein an alkyl group is as defined herein. Preferred aminoalkyl groups include —NH—C₁₋₆ aminoalkyl groups (that is, moieties of formula —NH—(C₁₋₆ alkyl)), or moieties of formula —N(C₁₋₆ alkyl)₂. Exemplary aminoalkyl groups include —NHCH₃, —NHCH₂CH₃, and —N(CH₃)₂.

“Cycloalkyl” as used herein refers to a monovalent group derived from a saturated, typically monocyclic, hydrocarbon. A cycloalkyl group may have, for instance, 3 to 12 carbon atoms but “cycloalkyl” typically refers to a C₃₋₁₀ cycloalkyl group. C₃₋₇ cycloalkyl groups are particularly preferred. Exemplary cycloalkyl groups include cyclopentyl and cyclohexyl groups.

“Cycloalkylene” as used herein refers to a divalent moiety derived from a saturated, typically monocyclic, hydrocarbon. A cycloalkylene may have, for instance, 3 to 12 carbon atoms but “cycloalkylene” typically refers to a C₃₋₁₀ cycloalkylene group. C₃₋₇ cycloalkylene groups are particularly preferred. Exemplary cycloalkylene groups include cyclopentylene and cyclohexylene groups.

“Cycloalkenyl” as used herein refers to a non-aromatic monovalent group derived from a typically monocyclic hydrocarbon comprising one or more carbon-carbon double bonds. Typically a cycloalkenyl group contains one carbon-carbon double bond. A cycloalkenyl group may have from 3 to 12 carbon atoms; “cycloalkenyl” typically refers to a C₄₋₁₀ cycloalkenyl group. C₅₋₇ cycloalkenyl groups are particularly preferred.

“Cycloalkenylene” as used herein refers to a non-aromatic divalent group derived from a typically monocyclic hydrocarbon comprising one or more carbon-carbon double bonds. Typically a cycloalkenylene group contains one carbon-carbon double bond. A cycloalkenylene group may have from 3 to 12 carbon atoms; “cycloalkenylene” typically refers to a C₄₋₁₀ cycloalkenylene group. C₅₋₇ cycloalkenylene groups are particularly preferred.

“Heterocycloalkyl” as used herein refers to a monovalent saturated ring comprising at least one heteroatom selected from oxygen, sulphur and nitrogen. Heterocycloalkyl groups typically comprise one, two or three heteroatoms each independently selected from oxygen, sulphur and nitrogen, usually one such heteroatom. Heterocycloalkyl rings may be monocyclic (e.g. piperidinyl) or polycyclic (e.g. decahydroquinolinyl). A heterocycloalkyl group typically comprises from 5 to 14 carbon atoms.

“Heterocycloalkylene” as used herein refers to a divalent saturated ring comprising at least one heteroatom selected from oxygen, sulphur and nitrogen. Heterocycloalkylene groups typically comprise one, two or three heteroatoms each independently selected from oxygen, sulphur and nitrogen, usually one such heteroatom. Heterocycloalkylene rings may be monocyclic or polycyclic. A heterocycloalkylene group typically comprises from 5 to 14 carbon atoms.

“Heterocycloalkenyl” as used herein refers to a non-aromatic monovalent ring derived from an unsaturated heterocycloalkenyl group. Heterocycloalkenyl groups comprise one or more carbon-carbon double bonds. Typically a heterocycloalkenyl group contains one carbon-carbon double bond. A heterocycloalkenyl group also comprises at least one heteroatom selected from oxygen, sulphur and nitrogen. Heterocycloalkenyl groups typically comprise one, two or three heteroatoms each independently selected from oxygen, sulphur and nitrogen, usually one such heteroatom. Heterocycloalkenyl rings may be monocyclic or polycyclic. A heterocycloalkenyl group typically comprises from 5 to 14 carbon atoms.

“Heterocycloalkenylene” as used herein refers to a non-aromatic divalent ring derived from an unsaturated heterocycloalkenyl group. Heterocycloalkenylene groups comprise one or more carbon-carbon double bonds. Typically a heterocycloalkenylene group contains one carbon-carbon double bond. A heterocycloalkenylene group also comprises at least one heteroatom selected from oxygen, sulphur and nitrogen. Heterocycloalkenylene groups typically comprise one, two or three heteroatoms each independently selected from oxygen, sulphur and nitrogen, usually one such heteroatom. Heterocycloalkenylene rings may be monocyclic or polycyclic. A heterocycloalkenylene group typically comprises from 5 to 14 carbon atoms.

“Aryl” as used herein refers to a monovalent unsaturated aromatic carbocyclic group which may be monocyclic (for instance a phenyl group) or polycyclic, having multiple condensed rings (e.g. a naphthyl group). An aryl group typically contains from 6 to 14 carbon atoms. A preferred aryl group is phenyl.

“Arylene” as used herein refers to a divalent unsaturated aromatic carbocyclic group which may be monocyclic (for instance a phenylene group) or polycyclic, having multiple condensed rings (e.g. a naphthylene group). An arylene group typically contains from 6 to 14 carbon atoms. A preferred arylene group is phenylene.

“Heteroaryl” as used herein refers to a monovalent aromatic heterocyclic group having at least one heteroatom selected from oxygen, sulphur and nitrogen. Heteroaryl groups typically comprise one, two or three heteroatoms each independently selected from oxygen, sulphur and nitrogen, usually one such heteroatom. Heteroaryl groups may be monocyclic or polycyclic and are typically monocyclic. A heteroaryl group typically comprises from 5 to 14 carbon atoms.

“Heteroarylene” as used herein refers to a divalent aromatic heterocyclic group having at least one heteroatom selected from oxygen, sulphur and nitrogen. Heteroarylene groups typically comprise one, two or three heteroatoms each independently selected from oxygen, sulphur and nitrogen, usually one such heteroatom. Heteroarylene groups may be monocyclic or polycyclic and are typically monocyclic. A heteroarylene group typically comprises from 5 to 14 carbon atoms.

The term “halogen” or “halo” as used herein is intended to include fluorine, chlorine, bromine and iodine atoms, typically fluorine, chlorine or bromine.

The term “heterocyclic moiety” as used herein refers to the bivalent, saturated, nitrogen-containing heterocyclic moiety present in linker L. The moiety is a cyclic moiety containing at least one nitrogen atom. Other heteroatoms may be present within the heterocycle, but typically the heterocyclic moiety contains only one or more nitrogen atoms as the heteroatom(s) present. The heterocyclic moiety typically comprises one, two or three heteroatoms. Thus, the heterocyclic moiety may be a heterocycloalkylene group as described herein wherein at least one nitrogen heteroatom is present.

The heterocyclic moiety is typically a 5- to 7-membered ring. The heterocyclic moiety is typically monocyclic. The heterocyclic moeity typically comprises from 3 to 14 carbon atoms, more usually from 4 to 14 carbon atoms; often from 4 to 6 carbon atoms.

Throughout, a dotted covalent line indicates a covalent bond linking the moiety carrying that bond to another moiety.

The term “degrader” as used herein indicates a chimeric molecule comprising a moiety capable of binding to NUDT5, a moiety capable of recruiting cellular destruction machinery, and a linker joining the two aforementioned moieties. A degrader is therefore a species which is capable of bringing NUDT5 into close proximity with cellular destruction machinery, leading to the destruction of NUDT5. A degrader is therefore distinct from an inhibitor which merely binds to NUDT5, rather than destroying it.

Reference to “a compound of the invention” or “compounds of the invention” encompasses compounds of formula (I), (IA) and (IB). Compounds of formula (I) and (IA) are particularly preferred.

### Linker L

The compounds of formula (I) comprise linker L. L is a linker comprising at least one heterocyclic moiety, said heterocyclic moiety being a bivalent, saturated, nitrogen-containing heterocyclic moiety. The or each heterocyclic moiety therefore comprises at least one nitrogen atom.

Where linker L includes more than one heterocyclic moiety, each such heterocyclic moeity may be the same or different (although each is a heterocyclic moeity as described herein). Typically, L may include one, two or three heterocyclic moieties. Preferably, L includes one or two heterocyclic moieties. Reference to “the heterocyclic moeity” hereafter may be taken to refer to “the or each heterocyclic moeity” where multiple heterocyclic moeities are present.

The heterocyclic moiety is bivalent, meaning that it is attached at two points to other species. These other species are be selected from the X group, the Y group, and another atom within linker L. The another atom within linker L is typically a carbon atom but could also be an oxygen atom, a nitrogen atom, or a sulphur atom. Thus, there are two atoms within each heterocyclic moiety which are covalently bonded to another species selected from the X group, the Y group, and another atom within linker L.

These said two atoms which are covalently bonded to another species may or may not be adjacent to one another within the heterocyclic moiety. Typically, these said atoms are not adjacent to one another within the heterocyclic moiety; this may confer a steric advantage. For instance, where the heterocyclic moiety is a 6-membered heterocyclic moiety, the said two atoms may be situated para- to one another within the heterocyclic moiety.

Generally, at least one of the said two atoms which are covalently bonded to another species is a nitrogen atom.

The heterocyclic moiety is saturated, meaning that it contains only single covalent bonds within the ring. The heterocyclic moiety is non-aromatic.

The heterocyclic moiety may or may not be monocyclic. For instance, the heterocyclic moiety may be bicyclic. Typically, however, the heterocyclic moiety is monocyclic.

The heterocyclic moiety comprises at least three atoms within the ring. The heterocyclic moiety may be, for instance, a 3- to 16-membered heterocyclic moiety. By this is meant that the heterocyclic moiety may comprise from 3 to 16 ring atoms (by “ring atoms” is meant the atoms which form the cycle; it does not include substituent atoms). The ring atoms include at least one nitrogen atom. Generally, the heterocyclic moiety is a 4- to 10-membered heterocyclic moiety. Preferably, the heterocyclic moiety is a 5- to 7-membered heterocyclic moiety, as these rings are particularly stable. Particularly preferably, the heterocyclic moiety is a 5- or a 6-membered heterocyclic moiety, and most preferably a 6-membered heterocyclic moiety.

The heterocyclic moiety may comprise one or more other heteroatoms, in addition to the at least one nitrogen atom, within the ring. For instance, the heterocyclic moiety may comprise one or more atoms selected from O and S within the ring. Where another heteroatom in addition to the nitrogen atom(s) is present, the heterocyclic moiety typically only comprises one such other heteroatom, either O or S, and preferably O.

In exemplary embodiments, the only heteroatom(s) within the ring of the heterocyclic moiety is the one or more nitrogen atoms.

The heterocyclic moiety comprises at least one nitrogen atom. For instance, the heterocyclic moiety may comprise one, two or three nitrogen atoms. Preferably, the heterocyclic moiety comprises one or two nitrogen atoms. Particularly preferably, the heterocyclic moiety comprises one or two nitrogen atoms and no other heteroatoms.

The heterocyclic moiety may be selected from any of the following, where the two dotted lines indicate a covalent bond attaching the heterocyclic moiety to X, Y or another atom within linker L:

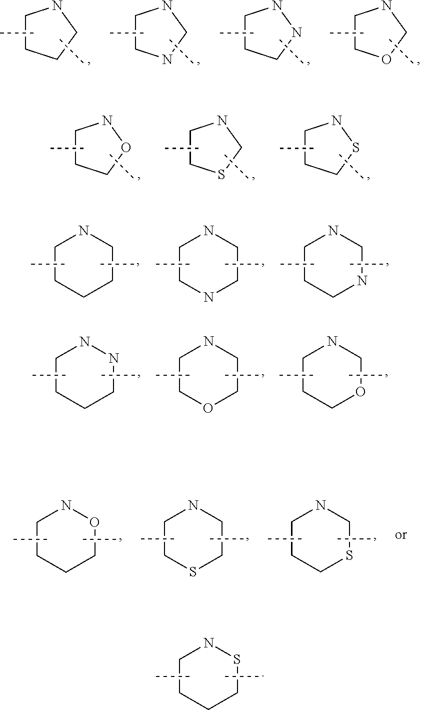

Preferably, the heterocyclic moiety is selected from:

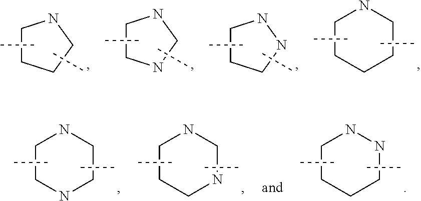

For example, the heterocyclic moiety may be selected from

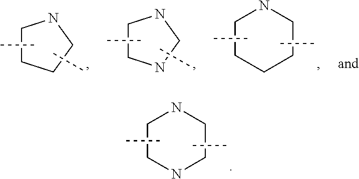

In particular, the heterocyclic moiety may be selected from

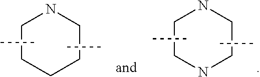

As noted above, each heterocyclic moiety comprises two atoms which are covalently bound to another species, the another species being selected from the X group, the Y group, and another atom within linker L. Generally, at least one of said two atoms is a nitrogen atom. Accordingly, the heterocyclic moiety may be selected from

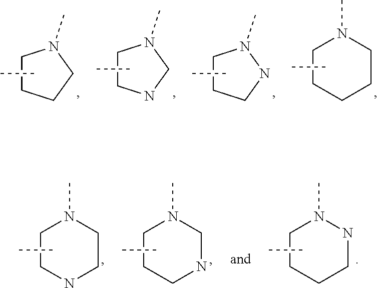

The heterocyclic moiety may be from the following moieties:

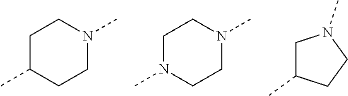

wherein, as explained above a dotted line indicates a covalent bond attaching the heterocyclic moiety to X, Y, or another portion of linker L (being another atom within linker L). Thus, often, the linker L comprises at least one moiety selected from any of the following:

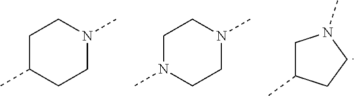

Preferably, the or each heterocyclic moiety is selected from the following:

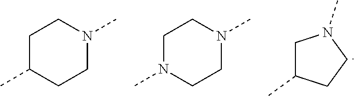

The linker L may consist entirely of the one or more heterocyclic moieties. More usually, however, the linker L additionally comprises one or more further bivalent moieties. The one or more further bivalent moieties are covalently bound to two species each selected from the X group, the Y group, a heterocyclic moiety and another bivalent group.

Suitable examples of the one or more further bivalent moieties include —O—, —NH—, alkylene, and —C(═Z)— where Z is S or O (preferably O). Exemplary bivalent moieties include —O—, —NH—, C₁-C₄ alkylene and —C(═Z)—, where Z is O or S.

Accordingly, linker L may comprise, in addition to the one or more heterocyclic moeities, one or more further bivalent moieties selected from —O—, —NH—, alkylene, and —C(═Z)— where Z is S or O. By way of example, linker L may comprise one, two, three, four or five further bivalent moieties, each independently selected from —O—, —NH—, alkylene, and —C(═Z)— where Z is S or O.

It should be noted that, although unsubstituted bivalent moieties are recited above, linker L may optionally be substituted. For instance, linker L may be substituted by 1 or 2 substituents each independently selected from halo, C₁-C₆ alkyl, or C₁-C₆ alkoxy. Alternatively, linker L may be unsubstituted.

In one aspect, linker L may comprise two heterocyclic moieties as described herein, optionally separated by a further bivalent moiety as described herein. For instance, linker L may comprise two heterocyclic moieties as described herein, intervened by one or more further bivalent moieties selected from —O—, —NH—, alkylene, and —C(═Z)— where Z is S or 0.

In such cases, preferably the two heterocyclic moieties are linked by 0, 1, 2 or 3 further bivalent moieties selected from —O—, —NH—, alkylene, and —C(═Z)— where Z is S or O; for example by 0 or 1 further such bivalent moieties.

Accordingly, in an exemplary aspect, linker L comprises or consists of a bicyclic moiety of formula (II):

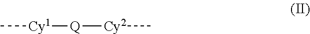

wherein:
Cy¹ and Cy² are each bivalent, saturated, nitrogen-containing heterocyclic moieties as described herein;
Q is selected from a covalent bond, C₁-C₆ alkylene; —NH—, —O—, —C(═Z)— where Z is O or S (preferably O), and any combination thereof; and
a dotted line indicates a covalent bond attaching the bicyclic moiety of formula (II) to X, Y, or another portion of linker L (another portion of linker L being another atom within linker L).

Preferably, Q is selected from a covalent bond, C₁-C₆ alkylene; —NH—, —O—, and —C(═O)—. More preferably, Q is selected from C₁-C₆ alkylene, —NH—, and —O—. For instance, Q may be methylene or ethylene.

Also preferably, Cy¹ and Cy² are 5- to 7-membered rings, particularly containing one or two heteroatoms and no other heteroatoms. Thus, in an exemplary aspect of formula (II), Cy¹ and Cy² are each 5- or 6-membered heterocyclic moieties, containing one or two nitrogen atoms and no other heteroatoms; and Q is selected from C₁-C₆ alkylene, —NH—, and —O—. For example, Cy¹ and Cy² may each be 6-membered heterocyclic moieties, containing one or two nitrogen atoms and no other heteroatoms; and Q is selected from methylene or ethylene.

One or more further bivalent moieties may be situated adjacent to Cy¹ and/or Cy² in the above formula (II). For instance, Cy¹ may be bound to a further bivalent moiety as described herein, and/or Cy² may be bound to a further bivalent moiety as described herein. For example, either Cy¹ or Cy² may be bound to a single further bivalent moiety, said single further bivalent moiety being selected from —O—, —NH—, alkylene, and —C(═Z)— where Z═S or O.

In an aspect, said further bivalent moiety may be —C(═O)— or —C(═S)—. For instance, linker L may comprise or consist of a bicyclic moiety of formula (III):

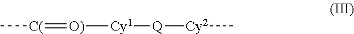

wherein Cy¹, Cy², Q and the dotted lines are as described herein. Preferably, Q is selected from a covalent bond, C₁-C₆ alkylene; —NH—, —O—, and —C(═Z)— where Z is O or S (preferably O). More preferably, Q is selected from C₁-C₆ alkylene; —NH—, and —O—. For instance, Q may be methylene or ethylene.

Also preferably, Cy¹ and Cy² are 5- to 7-membered rings, particularly containing one or two nitrogen atoms and no other heteroatoms. Thus, in an exemplary aspect of formula (III), Cy¹ and Cy² are each 5- or 6-membered heterocyclic moieties, containing one or two nitrogen atoms and no other heteroatoms; and Q is selected from C₁-C₆ alkylene, —NH—, and —O—. For example, Cy¹ and Cy² may each be 6-membered heterocyclic moieties, containing one or two nitrogen atoms and no other heteroatoms; and Q is selected from methylene or ethylene.

Formulae (II) and (III) concern linkers comprising two heterocyclic moieties. However, excellent degradation ability has also been observed in compounds of formula (I) having linkers containing one heterocyclic moiety. Accordingly, in another aspect, linker L may comprise a monocyclic moiety of any of formulae (IV), (V) or (VI):

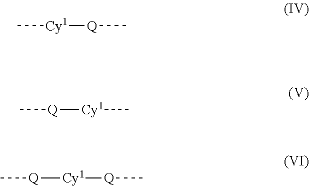

wherein Cy¹, Q and the dotted lines are as described herein. Preferably, Q is selected from a covalent bond, C₁-C₆ alkylene; —NH—, —O—, and —C(═Z)— where Z is O or S (preferably 0). More preferably, Q is selected from C₁-C₆ alkylene; —NH—, and —O—. For instance, Q may be methylene or ethylene.

Also preferably, Cy¹ is a 5- to 7-membered ring, particularly containing one or two nitrogen atoms and no other heteroatoms. Thus, in an exemplary aspect of formulae (IV), (V) and (VI), Cy¹ is a 5- or 6-membered heterocyclic moiety, containing one or two nitrogen atoms and no other heteroatoms; and Q is selected from C₁-C₆ alkylene, —NH—, and —O—. For example, Cy¹ may be a 6-membered heterocyclic moiety, containing one or two nitrogen atoms and no other heteroatoms; and Q is selected from methylene or ethylene.

Examples of linker L include the following. In this context, as there are no other atoms in the linker, a dotted line indicates a covalent bond to the X group or to the Y group.

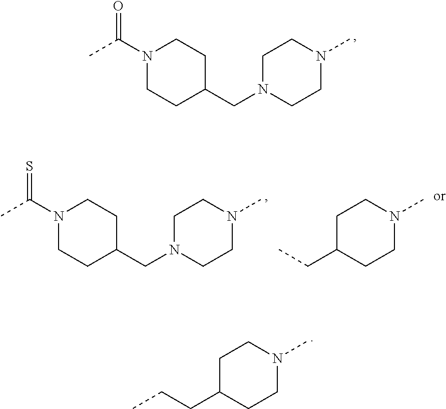

Particularly preferably, L is:

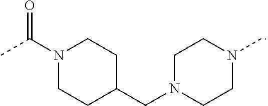

### Linker LA

A second group of linkers has also been identified which provide excellent NUDT5-degrading activity when used to couple X and Y groups. Accordingly, in another aspect, the invention provides a compound of formula (IA):

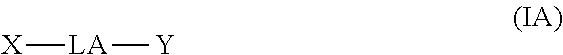

or a pharmaceutically acceptable salt thereof,
wherein X is a moiety capable of binding to NUDT5;
LA is a linker of formula:

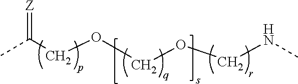

wherein
Z is O or S;
p, q, r and s are each integers independently selected from 1, 2, 3 or 4, each q being independently selected from 1, 2, 3 or 4 where s>1; and
Y is a moiety capable of binding to E3 ligase.

X and Y are as described herein.

Z is preferably O.

p may optionally be selected from 1, 2 or 3 and is most preferably 2.

q may optionally be selected from 1, 2 or 3 and is most preferably 2.

r may optionally be selected from 1, 2 or 3 and is most preferably 2.

s may optionally be selected from 1, 2 or 3.

Thus, p, q and r may all be selected from 1, 2 or 3; and in a particularly preferred aspect of linker LA, p, q and r are all 2.

Exemplary linkers of formula LA are shown below.

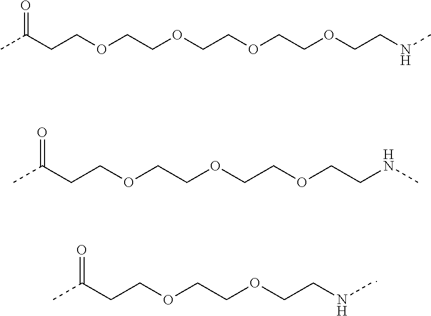

### Linker LB

The compound of formula (IB) comprises linker LB. LB is a bivalent moiety covalently bound to group X and group YB.

Generally, linker LB comprises at least one heteroatom. This may assist in conferring solubility on the compound of formula (IB). More preferably, therefore, linker LB comprises at least three heteroatoms. For instance, linker LB may comprise 3 to 10 heteroatoms each selected from O, N and S; preferably from O and N.

It is desirable that linker LB is sufficiently long to separate the moieties of formula X and YB to avoid interference, but that the linker LB is not so long that the moieties are too distant to bring NUDT5 and the cellular destruction machinery into close contact. Typically, the linker LB separates groups X and YB via a continuous chain of at least 3 atoms. Also typically, the linker LB separates groups X and YB by a continuous chain of no more than 30 atoms. For instance, the linker LB may separate groups X and YB by a continuous chain of from 2-30 atoms, preferably from 3-25 atoms, more preferably from 4-20 atoms; most preferably from 5-16 atoms. Where two differing possible continuous chains arise (for instance, as is possible where the linker LB comprises a cyclic moiety), the number of atoms linking X and YB is taken to be that in the shortest continuous chain of atoms linking X and YB.

Typically, linker LB comprises one or more groups selected from —O—, —S—, —NH—, —C(═Z)— where Z is O or S, alkylene, alkenylene, cycloalkylene, cycloalkenylene, heterocycloalkylene, heterocycloalkenylene, arylene, and heteroarylene. Preferably, LB comprises one or more groups selected from —O—, —S—, —NH—, —C(═Z)— where Z is O or S, alkylene, cycloalkylene, and heterocycloalkylene.

For example, linker LB may comprise one or more groups selected from —O—, —S—, —NH—, —C(═Z)— where Z is O or S, C₁₋₁₀ alkylene, C₂₋₁₀ alkenylene, C₅₋₇ cycloalkylene, C₅₋₇ cycloalkenylene, 5- to 7-membered heterocycloalkylene, 5- to 7-membered heterocycloalkenylene, C₆ arylene, and 5- to 7-membered heteroarylene. Preferably, linker LB comprises one or more groups selected from —O—, —S—, —NH—, —C(═Z)— where Z is O or S, C₁₋₁₀ alkylene, C₅₋₇ cycloalkylene, and 5- to 7-membered heterocycloalkylene.

Preferably, linker LB is a linker of formula L or formula LA as described herein. Thus, particularly preferably, linker LB is any of the following:

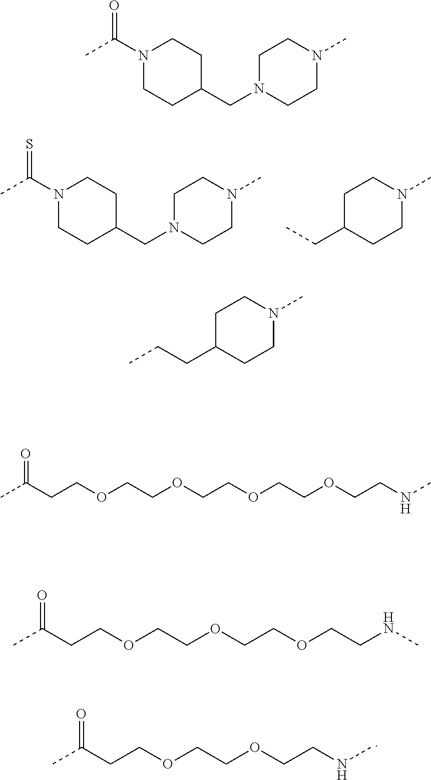

### Optional Substitution of Linkers

The linkers L, LA and LB described herein may optionally be substituted. By “substituted” is meant that any hydrogen atom present within linker L or LA may be replaced by a substituent group other than hydrogen.

The linkers L, LA and LB may be substituted at any suitable position. For instance, a nitrogen atom or a carbon atom may be substituted. A heterocyclic moiety and/or a further bivalent moiety may be substituted.

The linkers L, LA and LB may be unsubstituted (that is, substituted by 0 substituents) or substituted by one or more substituents. For instance, a linker L, LA or LB may be substituted by 0, 1, 2, 3, 4 or 5 substituents. Typically, the number of substituents is small or the linker is unsubstituted. Usually, therefore, the linker L, LA or LB is substituted by 0, 1 or 2 substituents. Preferably, L, LA or LB is substituted by 0 substituents (that is, it is unsubstituted).

Where present, a substituent does not detrimentally affect the ability of the compound of formula (I), (IA) or (IB) to form a ternary complex including NUDT5 and an active component of an cellular destruction pathway (such as an E3 ligase). The substituent(s) are typically small species.

Substituents may include halo, hydroxy (—OH), amino (—NH₂), alkyl, alkenyl, alkoxy, alkylthio, aminoalkyl, cycloalkyl, heterocycloalkyl, cycloalkenyl, heterocycloalkenyl, aryl or heteroaryl groups. Exemplary substituents include halo, hydroxy, amino, alkyl, alkoxy, or aminoalkyl groups. For instance, the linker L, LA or LB may be substituted by 0-5 substituents each independently selected from halo, hydroxy, amino, alkyl, alkoxy, or aminoalkyl. More particularly, the linker L, LA or LB may be substituted by 0, 1 or 2 substituents each independently selected from halo, hydroxy, amino, C₁-C₆ alkyl, C₁-C₆ alkoxy, —NH(C₁-C₆ alkyl) or —N(C₁-C₆ alkyl)₂.

Preferably, the level of substitution is low. Accordingly, L, LA or LB may be substituted by 0, 1 or 2 substituents each independently selected from halo, C₁-C₆ alkyl, or C₁-C₆ alkoxy; most preferably L, LA and LB are unsubstituted.

### Group X

X is a moiety capable of binding to NUDT5 (that is, the protein NUDT5). Typically, X is a moiety which can bind to NUDT5 with high efficiency. Compounds of formula (I), (IA) and (IB) all comprise an X group.

Previous workers have investigated inhibitors of NUDT5, which are species capable of binding to NUDT5. Such species are suitable X moieties. Accordingly, X may comprise an inhibitor of NUDT5. For instance, X may comprise a known inhibitor of NUDT5, or a variant thereof.

A suitable class of X moieties is based on Ibrutinib, and derivatives thereof. Accordingly, X may have the general formula:

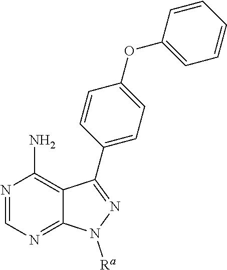

wherein R^{a} is a substituent. Either R^{a} is a monovalent substituent, and any hydrogen atom on the skeleton shown above is replaced by a covalent bond to the linker L or LA; or, more typically, R^{a} is a bivalent substituent which binds to the skeleton as shown above and to the linker, L or LA.

The inventors have found that considerable variability is tolerated at the R^{a} position, without affecting the ability of the group to bind to NUDT5. Accordingly, wide variation may be tolerated at the R^{a} position.

Where R^{a} is a monovalent substituent, R^{a} may for instance be hydrogen, alkyl, alkenyl, cycloalkyl, cycloalkenyl, heterocycloalkyl or heterocycloalkenyl group.

Where R^{a} is a bivalent substituent, R^{a} may be a covalent bond, alkylene, alkenylene, cycloalkylene, cycloalkenylene, heterocycloalkylene or heterocycloalkenylene group.

Typically, R^{a} is a bivalent substituent. For example, R^{a} may be a covalent bond, C₁-C₆ alkylene, C₂-C₆ alkenylene, C₅-C₇ cycloalkylene, C₅-C₇ cycloalkenylene, 5- to 7-membered heterocycloalkylene or 5- to 7-membered heterocycloalkenylene. In particular, R^{a} may be C₅-C₇ cycloalkylene or 5- to 7-membered heterocycloalkylene. Particularly, R^{a} may be a or 5- to 7-membered heterocycloalkylene containing a single nitrogen heteroatom, and most preferably a 6-membered heterocycloalkylene containing a single nitrogen heteroatom.

A particularly preferred class of X moieties has the following formula:

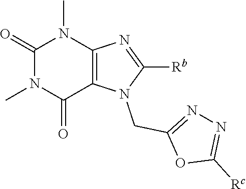

wherein
either R^{b} is a divalent moiety bound to the linker L or LA, or R^{b} is a monovalent moiety and any hydrogen atom in the skeleton shown above is replaced by a covalent bond to linker L or LA; and
R^{c} is a monovalent aryl or heteroaryl group.

Where R^{b} is a monovalent substituent, R^{b} may for instance be hydrogen, halogen, hydroxy, —SH, alkyl, alkenyl, alkoxy, alkylthio, cycloalkyl, cycloalkenyl, heterocycloalkyl or heterocycloalkenyl group.

Where R^{b} is a bivalent substituent, R^{b} may be a covalent bond, —O—, —S—, alkylene, alkenylene, alkyleneoxy, alkylenethio, alcycloalkylene, cycloalkenylene, heterocycloalkylene or heterocycloalkenylene group.

Typically, R^{b} is a bivalent substituent. For example, R^{b} may be a covalent bond, —O—, —S—, C₁-C₆ alkylene, C₂-C₆ alkenylene, C₁-C₆ alkyleneoxy, C₁-C₆ alkylenethio, C₅-C₇ cycloalkylene, C₅-C₇ cycloalkenylene, 5- to 7-membered heterocycloalkylene or 5- to 7-membered heterocycloalkenylene. In particular, R^{b} may be C₅-C₇ cycloalkylene or 5- to 7-membered heterocycloalkylene. Particularly, R^{b} may be a or 5- to 7-membered heterocycloalkylene containing a one or two nitrogen heteroatom, and most preferably a 6-membered heterocycloalkylene containing one or two nitrogen heteroatoms.

R^{c} is typically a 5- to 7-membered aryl or heteroaryl group, and is preferably a phenyl group.

R^{c} may be optionally substituted, as explained below in more detail. For instance, R^{c} may be substituted by one, two or three substituents each independently selected from halogen, alkyl, and alkoxy. In particular, R^{c} may be substituted by one, two or three substituents each independently selected from F, Cl, Br, C₁₋₄ alkyl and C₁₋₄ alkoxy. Particularly preferred examples of R^{c} include:

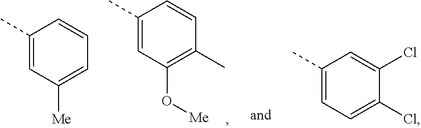

wherein a dotted line indicates a covalent bond to the rest of the X group.

The X groups described herein (including R^{a}, R^{b} and R^{c}) may optionally be substituted. By “substituted” is meant that any hydrogen atom present within the X group (including R^{a}, R^{b} and R^{c}) may be replaced by a substituent group other than hydrogen.

The X groups described herein may be unsubstituted (that is, substituted by 0 substituents) or substituted by one or more substituents. For instance, an X group may be substituted by 0, 1, 2, 3, 4 or 5 substituents. Typically, the number of substituents is small or the X group is unsubstituted. Usually, therefore, the X group is substituted by 0, 1 or 2 substituents. Preferably, the X group is substituted by 0 substituents (that is, it is unsubstituted).

Where present, a substituent does not detrimentally affect the ability of the X group to bind to NUDT5. The substituent(s) are typically small species.

Substituents may include halo, hydroxy (—OH), amino (—NH₂), alkyl, alkenyl, alkoxy, alkylthio, aminoalkyl, cycloalkyl, heterocycloalkyl, cycloalkenyl, heterocycloalkenyl, aryl or heteroaryl groups. Exemplary substituents include halo, hydroxy, amino, alkyl, alkoxy, or aminoalkyl groups. For instance, the X group may be substituted by 0-5 substituents each independently selected from halo, hydroxy, amino, alkyl, alkoxy, or aminoalkyl. More particularly, the X group may be substituted by 0, 1 or 2 substituents each independently selected from halo, hydroxy, amino, C₁-C₆ alkyl, C₁-C₆ alkoxy, —NH(C₁-C₆ alkyl) or —N(C₁-C₆ alkyl)₂. Exemplary substituents are C₁-C₄ alkyl.

Preferably, the level of substitution is low. Accordingly, the X group may be substituted by 0, 1 or 2 substituents each independently selected from halo, C₁-C₆ alkyl, or C₁-C₆ alkoxy; most preferably the X group is unsubstituted.

X may preferably be selected from:

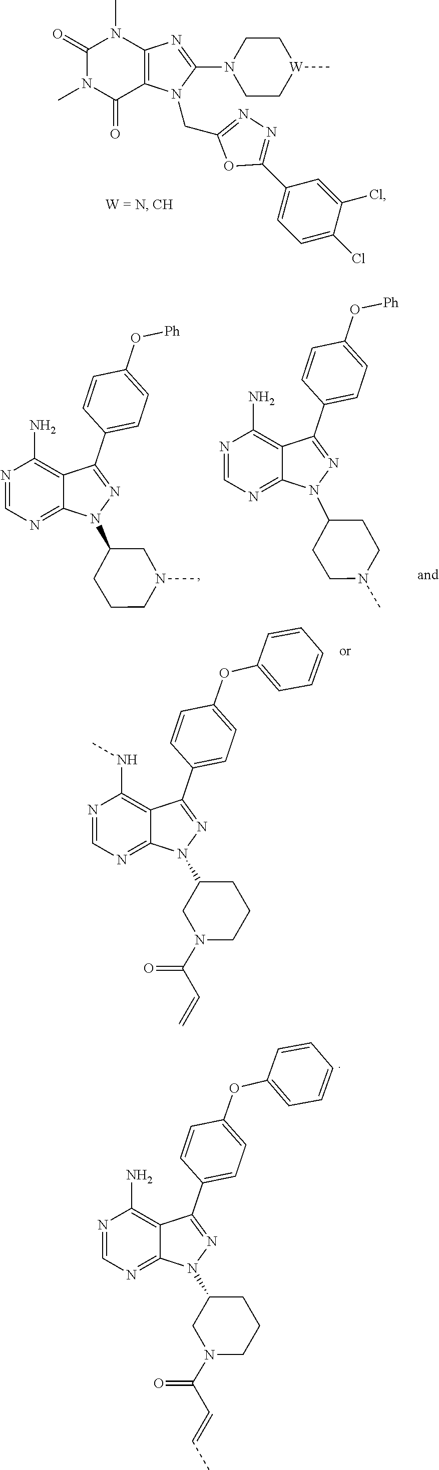

Group W may be N or CH. More concisely, this may be expressed as “W═N, CH” or “W═N, C”. Where W is not N, W is CH. Preferably, W is N.

Still more preferably, X may be selected from:

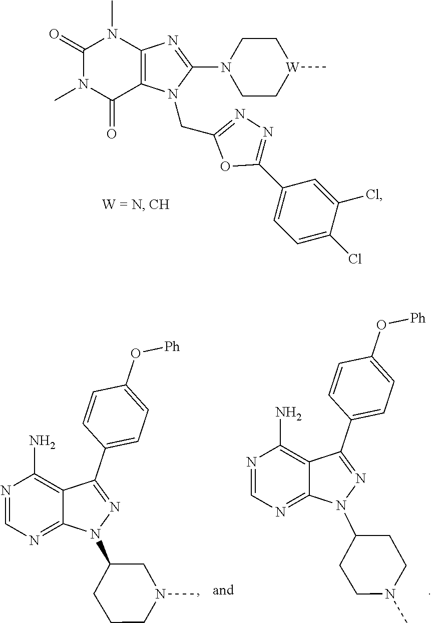

Most preferably, X is

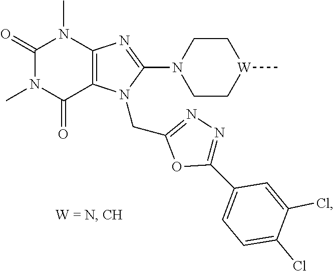

particularly where W═N.

### Group Y

Y is a moiety capable of binding to E3 ligase, typically to cereblon.

E3 ligases are species which promote ubiquitination and hence degradation of proteins. Accordingly, bringing a protein into close proximity with an E3 ligase has been found to be a possible mechanism of promoting degradation of that protein. This activity is utilised in the compounds described herein.

The E3 family is a large family, believed to contain over 600 proteins. Accordingly, these ligases are be targeted and bound by a wide variety of molecular structures. A variety of structures are therefore possible within the Y group. A review of such structures is performed in the article “Discovery of E3 ligase Ligands for Target Protein Degradation” (Molecules 2022, 27, 6615, Lee et al.), the entirety of which is incorporated by reference. Particular sub-groups of Y group structures may be conveniently grouped by the particular E3 ligase(s) that they are capable of binding to, and will be discussed in turn below.

### 1. CRBN-Targeting Y Groups

A particularly preferred type of Y group is a group capable of binding to the E3 ligase cereblon (also referred to as CRBN). In a preferred aspect, therefore, the Y group may be a CRBN ligand. Exemplary Y groups which are capable of binding to CRBN include the following, where a dotted line indicates the point of attachment to linker L or LA:

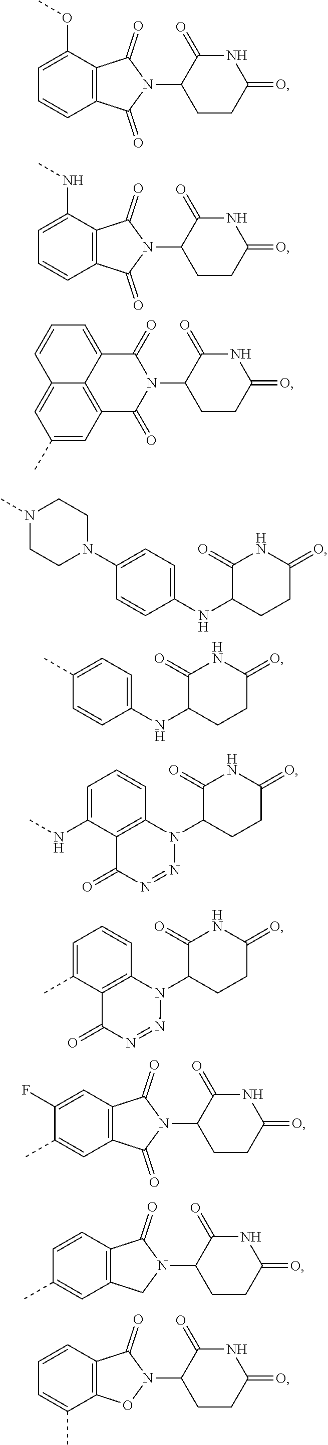

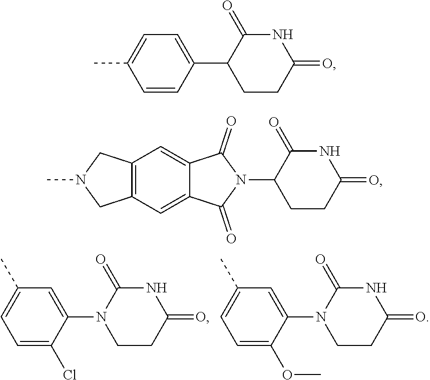

More generally, preferred Y groups include the following:

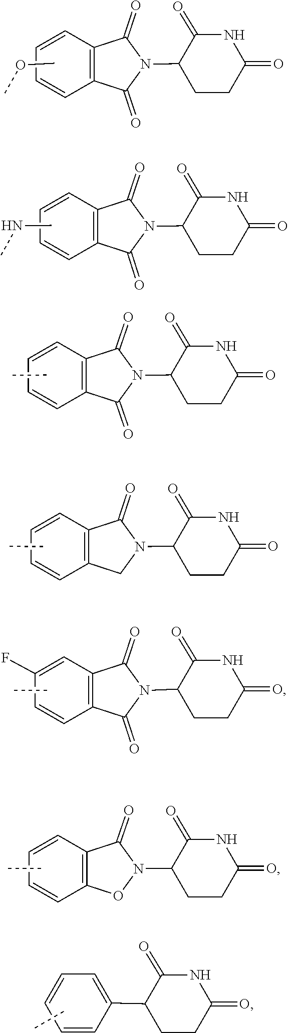

Preferably, Y is:

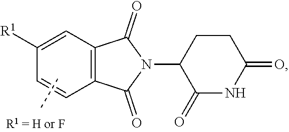

particularly preferably wherein R¹ is H.

### 2. VHL-Targeting Y Groups

Another suitable type of Y groups is a group capable of binding to the E3 ligase von Hippel-Lindau protein (also referred to as VHL). In an aspect, therefore, the Y group may be a VHL ligand. Exemplary Y groups which are capable of binding to VHL include the following, where a dotted line indicates the point of attachment to linker L or LA.

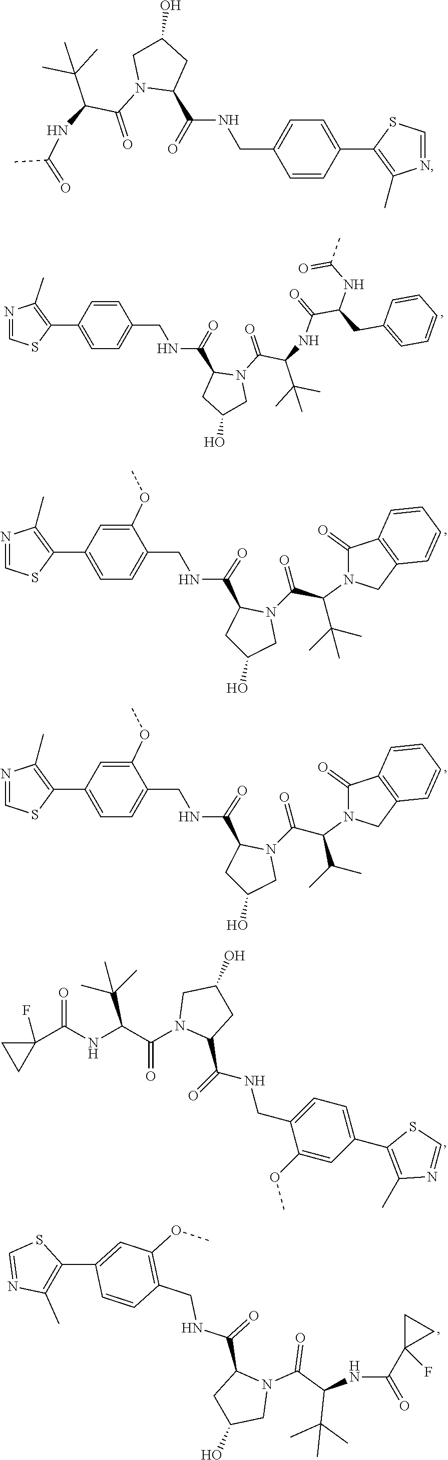

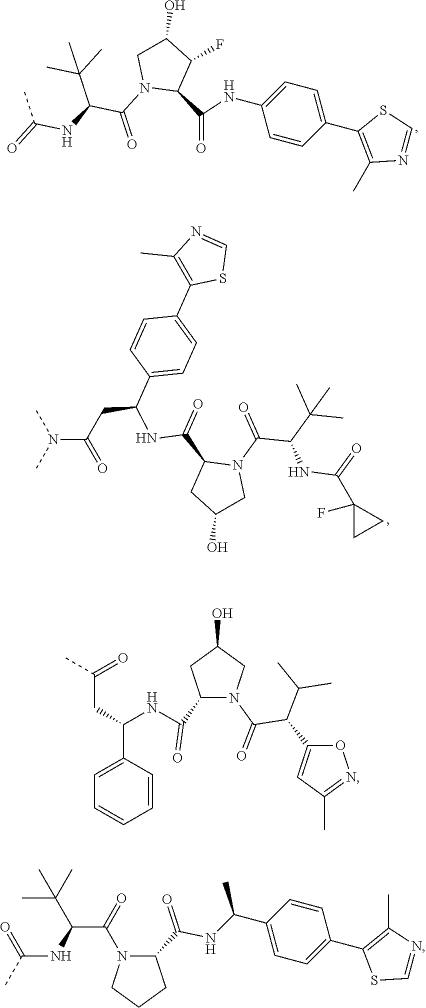

Where Y is a VHL ligand, a particularly preferred Y group is:

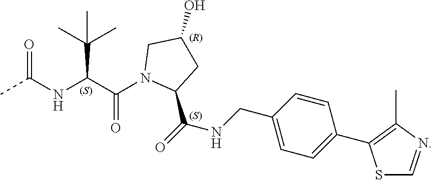

### 3. IAP-Targeting Y Groups

Another suitable type of Y groups is a group capable of binding to an “inhibitor of apoptosis protein” (also referred to as an IAP protein). In an aspect, therefore, the Y group may be an IAP ligand. Exemplary Y groups which are capable of binding to an IAP protein include the following, where a dotted line indicates the point of attachment to linker L, LA or LB.

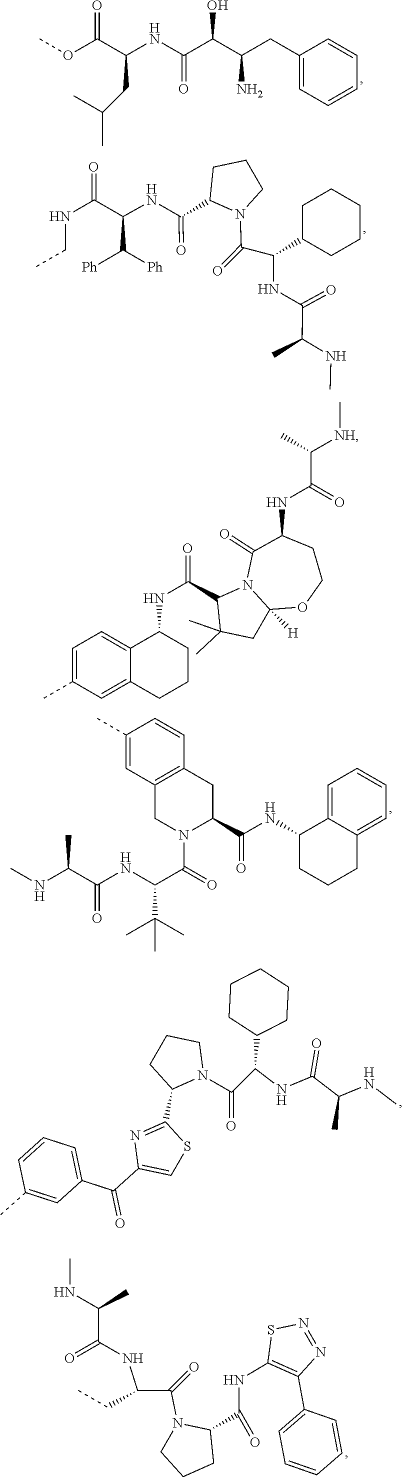

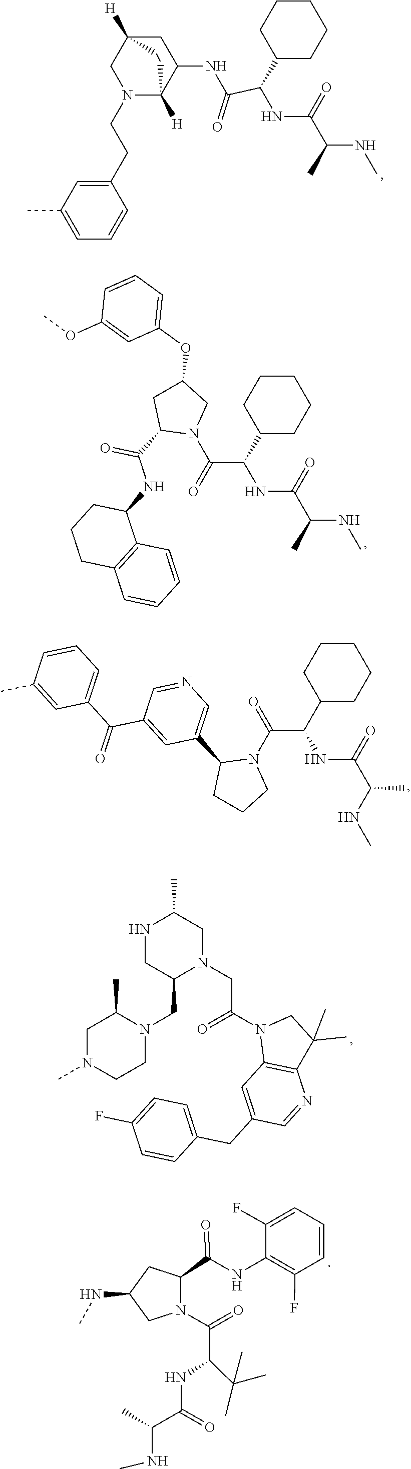

### 4. MDM2-Targeting Y Groups

Another suitable type of Y groups is a group capable of binding to the mouse double minute 2 homolog (MDM2). In an aspect, therefore, the Y group may be an MDM2 ligand. Exemplary Y groups which are capable of binding to MDM2 include the following, where a dotted line indicates the point of attachment to linker L, LA or LB.

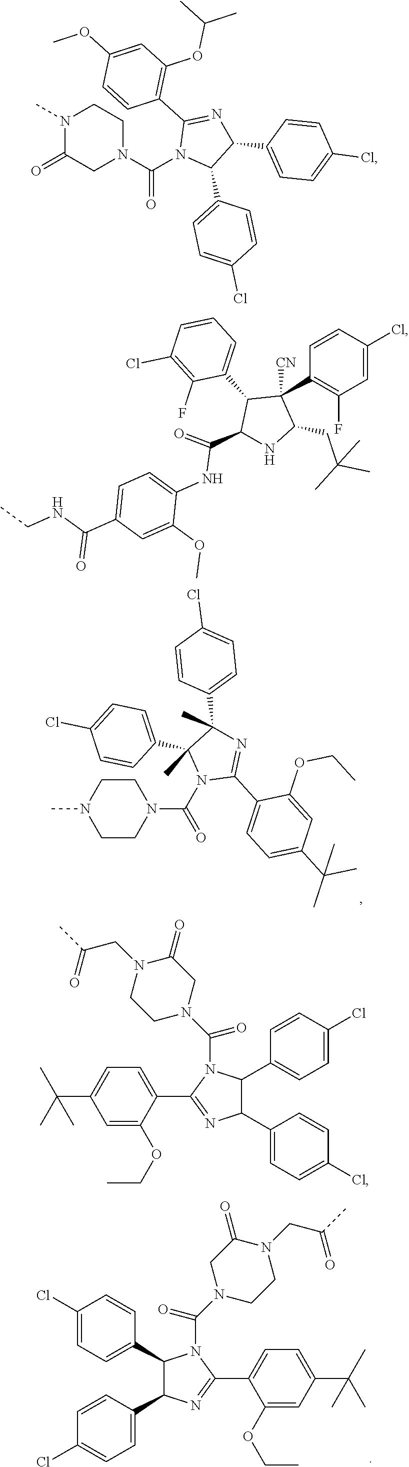

### 5. DCAF-Targeting Y Groups

Another suitable type of Y groups is a group capable of binding to DCAF proteins. DCAF proteins include DCAF15, DCAF16, and DCAF11. In an aspect, therefore, the Y group may be a DCAF ligand. Exemplary Y groups which are capable of binding to one or more DCAF proteins include the following, where a dotted line indicates the point of attachment to linker L, LA or LB.

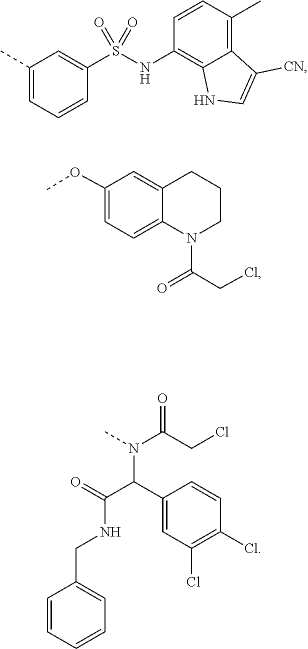

### 6. RNF-Targeting Y Groups

Another suitable type of Y groups is a group capable of binding to RNF proteins, particularly RNF4 and RNF114. In an aspect, therefore, the Y group maybe an RNF ligand, particularly an RNF4 ligand or an RNF114 ligand. Exemplary Y groups which are capable of binding to one or more RNF proteins include the following, where a dotted line indicates the point of attachment to linker L, LA or LB.

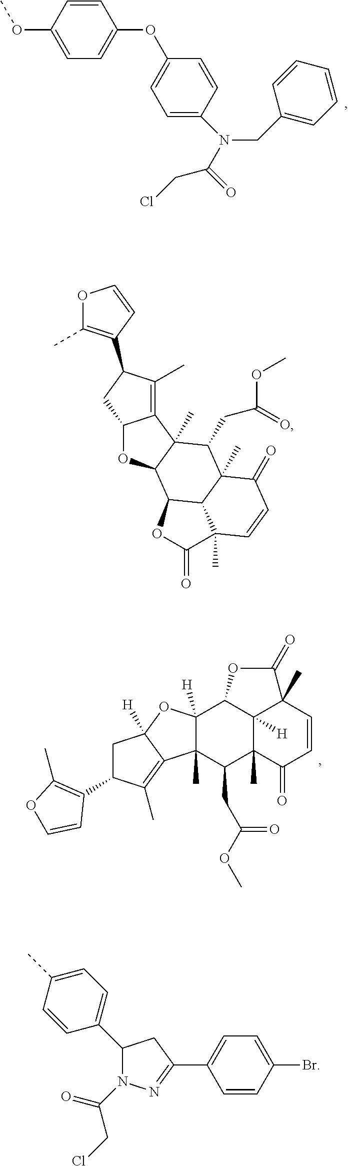

### 7. Ahr-Targeting Y Groups

Another suitable type of Y groups is a group capable of binding to the aryl hydrocarbon receptor (AhR) E3 ligase complex. In an aspect, therefore, the Y group may be an AhR ligand. An exemplary AhR ligand Y group is the following.

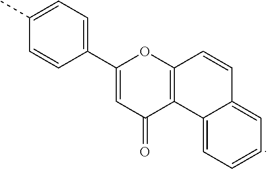

### 8. FEM1B-Targeting Y Groups

Another suitable type of Y groups is a group capable of binding to the CUL2 E3 ligase FEM1B. In an aspect, therefore, the Y group may be an FEM1B ligand. Exemplary Y groups which are capable of binding to FEM1B include the following.

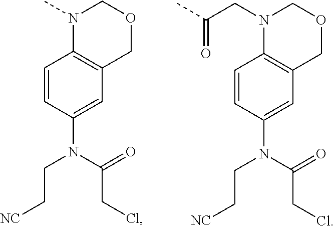

### 9. KEAP1-Targeting Y Groups

Another suitable type of Y groups is a group capable of binding to Kelch-like ECH-associated protein-1 (KEAP1). In an aspect, therefore, the Y group may be a KEAP1 ligand. Exemplary Y groups which are capable of binding to KEAP1 include the following.

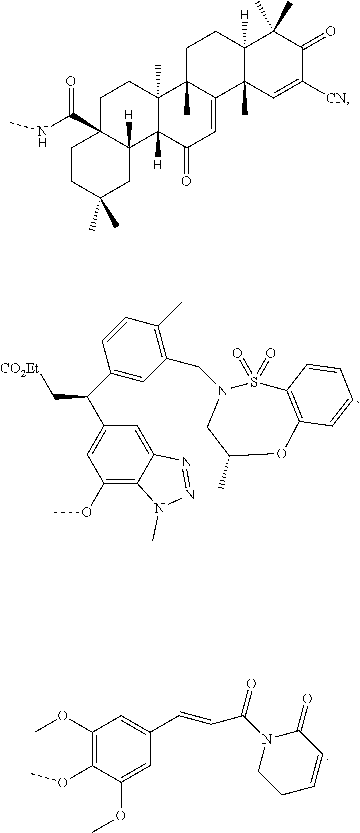

Preferably, Y is selected from:

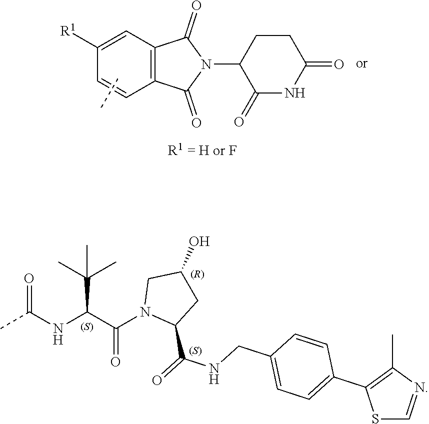

In a particularly preferred aspect, X is:

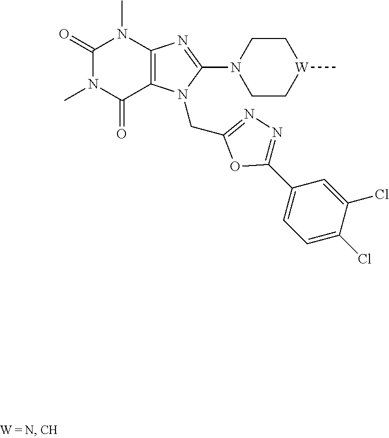

### and Y is:

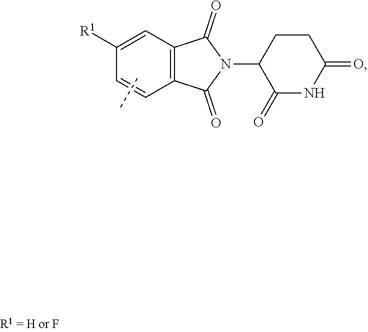

preferably wherein W is N and R¹ is H.

The Y groups described herein may optionally be substituted. By “substituted” is meant that any hydrogen atom present within the Y group may be replaced by a substituent group other than hydrogen.

For instance, a Y group may be substituted by 0, 1, 2, 3, 4 or 5 substituents. Typically, the number of substituents is small or the Y group is unsubstituted. Usually, therefore, the Y group is substituted by 0, 1 or 2 substituents; most preferably 0 substituents.

Where present, a substituent does not detrimentally affect the ability of the Y group to bind to an E3 ligase. The substituent(s) are typically small species.

Substituents may include halo, hydroxy (—OH), amino (—NH₂), alkyl, alkenyl, alkoxy, alkylthio, aminoalkyl, cycloalkyl, heterocycloalkyl, cycloalkenyl, heterocycloalkenyl, aryl or heteroaryl groups. Exemplary substituents include halo, hydroxy, amino, alkyl, alkoxy, or aminoalkyl groups. For instance, the Y group may be substituted by 0-5 substituents each independently selected from halo, hydroxy, amino, alkyl, alkoxy, or aminoalkyl. More particularly, the Y group may be substituted by 0, 1 or 2 substituents each independently selected from halo, hydroxy, amino, C₁-C₆ alkyl, C₁-C₆ alkoxy, —NH(C₁-C₆ alkyl) or —N(C₁-C₆alkyl)₂. Exemplary substituents are C₁-C₄ alkyl.

Preferably, the level of substitution is low. Accordingly, the Y group may be substituted by 0, 1 or 2 substituents each independently selected from halo, C₁-C₆ alkyl, or C₁-C₆ alkoxy; most preferably the Y group is unsubstituted.

### Group YB

YB is a moiety capable of recruiting cellular destruction machinery, except that YB is not a moiety capable of binding to VHL or CRBN.

There are numerous mechanisms within living cells which destroy proteins. One example is proteasomes, which are protein complexes which degrade proteins. This process is mediated by proteins particularly including the E3 ligases and E2 ligases, which are involved in a complicated mechanism which attaches ubiquitin to proteins, thus “flagging” them for degradation by proteasomes. Another example is the autophagy system, which delivers proteins for destruction to lysosomes, vacuoles or autophagosomes which destroy the protein. These mechanisms are collectively referred to, for convenience, as “cellular destruction pathways” or “cellular protein destruction pathways”. “Cellular destruction machinery” or “cellular protein destruction machinery” comprises the active agents in living cells which degrade proteins.

Thus, a moiety capable of recruiting cellular destruction machinery is a moiety which binds to an active component within a cellular destruction pathway. These active components are not particularly limited. They may be, for instance, the species which directly degrades a protein (such as a proteasome). Alternatively, an active component of a cellular destruction pathway may be, for example, an which “flags” a protein for destruction, either by itself binding to the protein or by adding a marker to the protein which will lead to its processing by the cellular destruction pathway.

Specifically, therefore, YB is a species which binds to an active component of a cellular destruction pathway, thus enabling the formation of a ternary complex comprising the degrader, NUDT5, and said active component of that cellular destruction pathway. This ultimately leads to the destruction of the NUDT5 protein by the cellular destruction pathway.

Active components of cellular destruction pathways, and small molecules which bind thereto, are known. In principle, derivatives of any of these small molecules are suitable as YB groups. Particular examples of YB groups are discussed below.

### 1. Hydrophobic Tag

YB may be a hydrophobic tag. A hydrophobic tag may be any hydrophobic moiety, and is not particularly limited. The hydrophobic tag may itself destabilise NUDT5 by greatly reducing its solubility when bound to the degrader. This can lead to the formation of aggregates which are destroyed by cellular destruction machinery. Alternatively, the hydrophobic tag may bind to members of the HSP70 family, particularly HSP40 and HSP70. These proteins cause ubiquitination of other proteins, and subsequent destruction thereof by proteasomes. Suitable hydrophobic tags are described in “Degradation of proteins by PROTACs and other strategies”, Wang et al., Acta Pharmaceutica Sinica B 2020, 10(2), pp 207-238, which is incorporated herein by reference.

Exemplary YB groups in this category include the following.

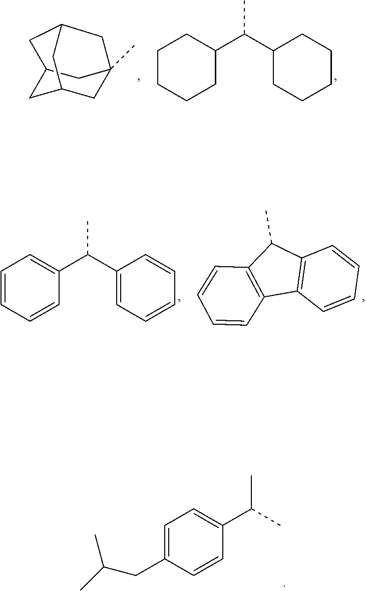

### 2. Autophagy-Targeting Moiety

YB may be an autophagy-targeting moiety; that is, a group which binds to an active component in the autophagy system. In particular, YB may be a group which binds to an active component of the autophagy-lysosome system. Suitable targets in this regard are discussed in detail in “Targeted protein degradation: mechanisms, strategies and application”, Zhao et al., Signal transduction and Targeted Therapy (2022), 7:113, which is incorporated herein by reference.

For instance, YB may be an AUTAC group, comprising a cGMP-based degradation tag:

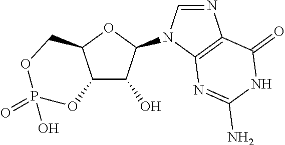

wherein any H is replaced by a covalent bond to the linker LB. YB may similarly be any derivative of this cGMP group.

In another example, YB may be an ATTEC group, comprising a moiety which binds to LC3.

In another example, YB may be an AUTOTAC group, comprising a moiety which binds to p62.

In another example, YB may be a CMA-based group, comprising a cell membrane penetration sequence and a CMA-targeting motif.

### 3. Moiety Capable of Binding to an E2 Ligase

YB may be a moiety capable of binding to E2 ligase.

A suitable example of a small molecule known to bind to an E2 ligase (particularly UBE2D) is described in “Targeted protein degradation through E2 recruitment”, Forte et al., BioRxiv (incorporated by reference). Accordingly, the YB group may be any derivative of this species, referred to as “EN67”. An exemplary YB group is shown below.

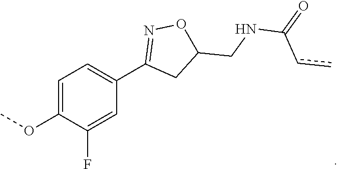

### 4. Moiety Capable of Binding to an E3 Ligase

YB may be any moiety capable of binding to E3 ligase, other than a moiety capable of binding to VHL or CRBN. These groups have been discussed in detail in connection with group Y, above. That discussion (except insofar as it relates to moieties capable of binding to VHL or CRBN) applies equally to the YB group.

### 5. Proteasome Recruiter

YB may be a proteasome recruiter, by which is meant a species which may bind directly to a proteasome.

One example of a suitable YB group is a Boc₃Arg ligand, which is believed to bind directly to the 20S proteasome (although the specific cellular destruction pathway has yet to be elucidated). Thus, the YB group may be the following, or any derivative thereof:

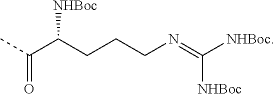

Another suitable type of YB group in this category is a macrocyclic group, which bond to the 26S proteasome. Such groups are described in “Targeted degradation via direct 26S proteasome recruitment”, Bashore et al., Nature Chemical Biology, Vol. 19, 2023, pp 55-63. Particular examples of such groups include:

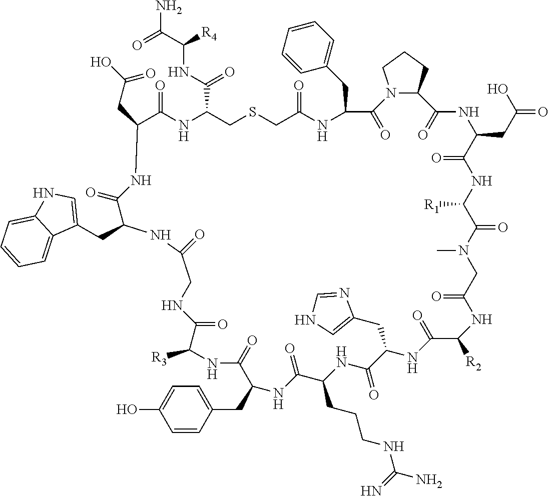

wherein any H is replaced by a covalent bond to linker LB; and
wherein R₁ is selected from isopropyl and —CH₂OCH₂C≡CH; R₂ is selected from isobutyl and —CH₂OCH₂C≡CH, R₃ is selected from

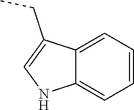

and —CH₂OCH₂C≡CH, R₄ is selected from H and —CH₂C—C≡CH.

It should be noted that some YB groups may fall into more than one of the above categories.

The YB groups described herein may optionally be substituted. By “substituted” is meant that any hydrogen atom present within the YB group may be replaced by a substituent group other than hydrogen.

For instance, a YB group may be substituted by 0, 1, 2, 3, 4 or 5 substituents. Typically, the number of substituents is small or the YB group is unsubstituted. Usually, therefore, the YB group is substituted by 0, 1 or 2 substituents; most preferably 0 substituents.

Where present, a substituent does not detrimentally affect the ability of the Y group to bind to the active component of the cellular destruction pathway of interest. The substituent(s) are typically small species.

Substituents may include halo, hydroxy (—OH), amino (—NH₂), alkyl, alkenyl, alkoxy, alkylthio, aminoalkyl, cycloalkyl, heterocycloalkyl, cycloalkenyl, heterocycloalkenyl, aryl or heteroaryl groups. Exemplary substituents include halo, hydroxy, amino, alkyl, alkoxy, or aminoalkyl groups. For instance, the YB group may be substituted by 0-5 substituents each independently selected from halo, hydroxy, amino, alkyl, alkoxy, or aminoalkyl. More particularly, the YB group may be substituted by 0, 1 or 2 substituents each independently selected from halo, hydroxy, amino, C₁-C₆ alkyl, C₁-C₆ alkoxy, —NH(C₁-C₆ alkyl) or —N(C₁-C₆ alkyl)₂. Exemplary substituents are C₁-C₄ alkyl.

Preferably, the level of substitution is low. Accordingly, the YB group may be substituted by 0, 1 or 2 substituents each independently selected from halo, C₁-C₆ alkyl, or C₁-C₆ alkoxy; most preferably the YB group is unsubstituted.

Where Y is YB, X is as described herein. Thus, where Y is YB, X is most preferably

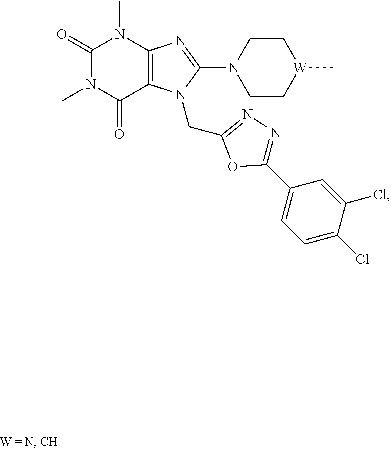

particularly preferably where W═N.

### Compounds of Formula (I) and (IA)

The degrader compounds of formula (I) and (IA) described herein comprise an X group (as described above, capable of binding to NUDT5) and a Y group (as described above, capable of binding to an E3 ligase) joined by a linker L or LA (also as described above). Accordingly, the compounds of formula (I) and formula (IA) are degraders which are capable of forming a ternary complex comprising NUDT5, an E3 ligase, and the compound of formula (I) or (IA) itself. Thus, the invention also provides a binary complex comprising any of the compounds of formula (I) or (IA) described herein, together with NUDT5 or an E3 ligase (particularly VHL or CRBN). Further, the invention also provides a ternary complex comprising any of the degrader compounds of formula (I) or (IA) described herein, together with NUDT5 and an E3 ligase (particularly VHL or CRBN).

Particularly preferred compounds of formula (I) include any of the following, and any pharmaceutically acceptable salt thereof:

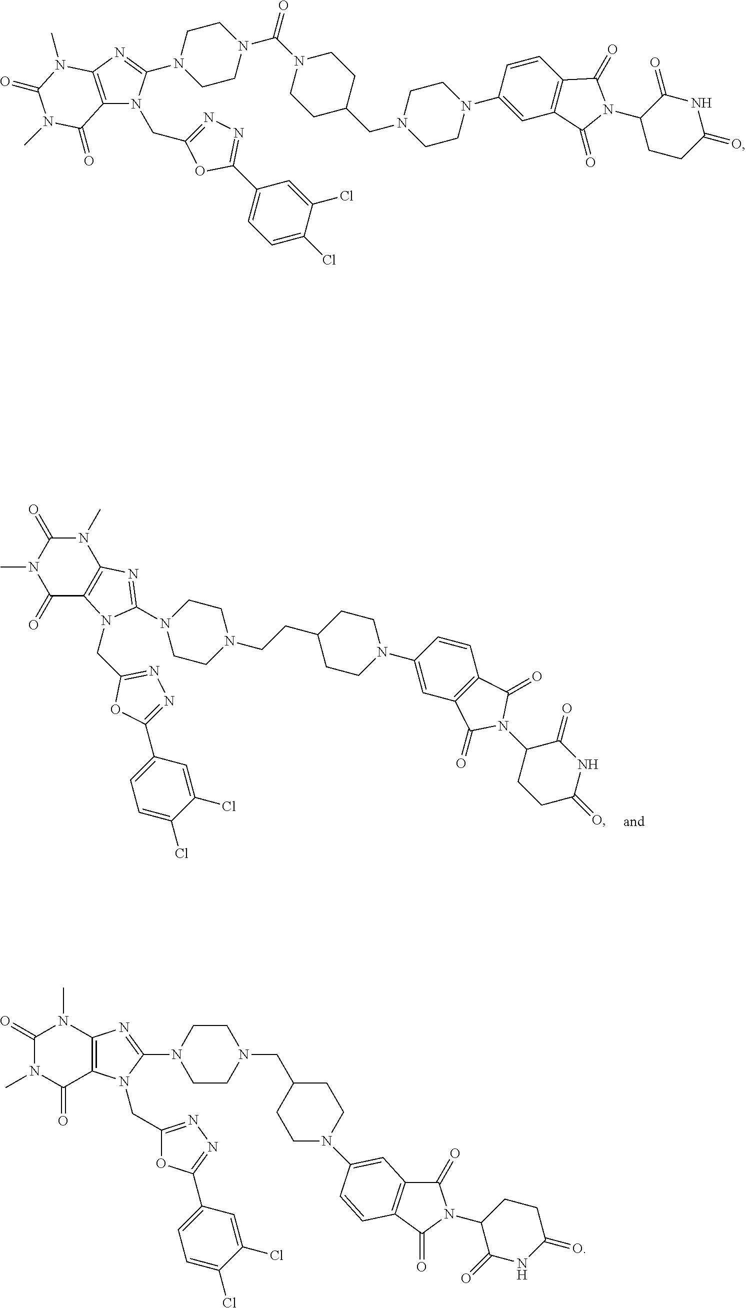 [SMILES: Cn1c(=O)c2c(nc(N3CCN(C(=O)N4CCC(CN5CCN(c6ccc7c(c6)C(=O)N(C6CCC(=O)NC6=O)C7=O)CC5)CC4)CC3)n2Cc2nnc(-c3ccc(Cl)c(Cl)c3)o2)n(C)c1=O ; Cn1c(=O)c2c(nc(N3CCN(CCC4CCN(c5ccc6c(c5)C(=O)N(C5CCC(=O)NC5=O)C6=O)CC4)CC3)n2Cc2nnc(-c3ccc(Cl)c(Cl)c3)o2)n(C)c1=O ; Cn1c(=O)c2c(nc(N3CCN(CC4CCN(c5ccc6c(c5)C(=O)N(C5CCC(=O)NC5=O)C6=O)CC4)CC3)n2Cc2nnc(-c3ccc(Cl)c(Cl)c3)o2)n(C)c1=O]

Particularly preferred compounds of formula (IA) include any of the following, and any pharmaceutically acceptable salt thereof:

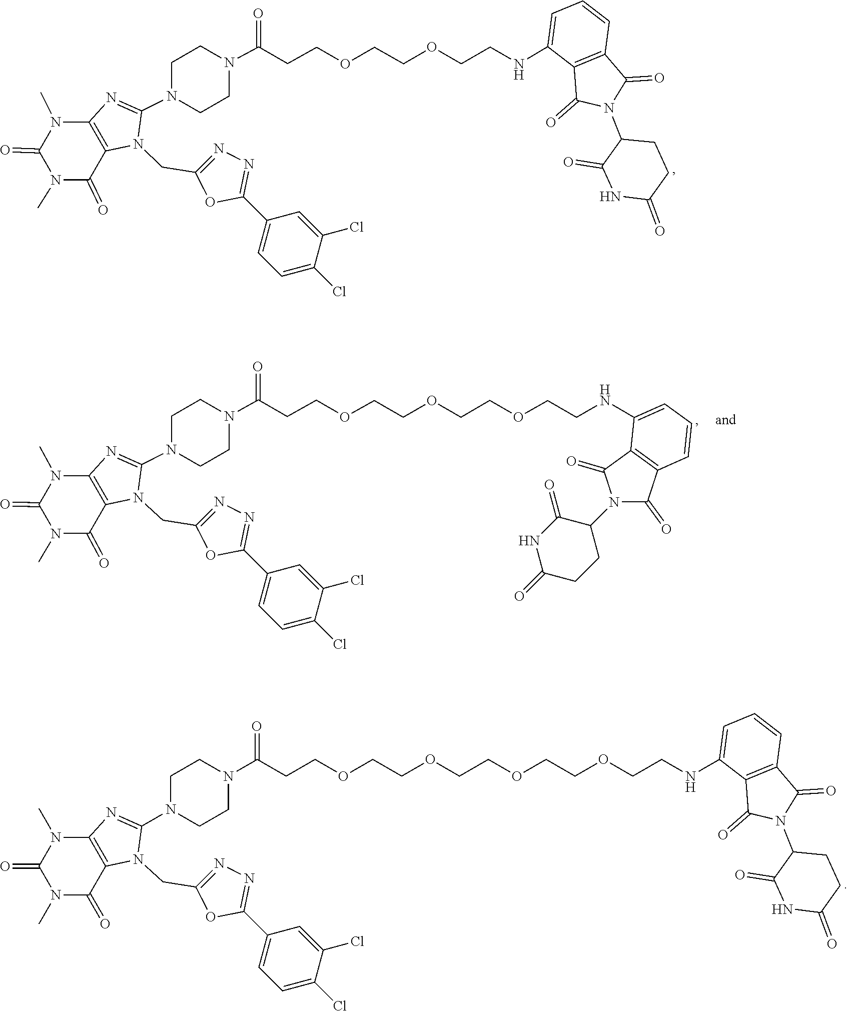 [SMILES: Cn1c(=O)c2c(nc(N3CCN(C(=O)CCOCCOCCNc4cccc5c4C(=O)N(C4CCC(=O)NC4=O)C5=O)CC3)n2Cc2nnc(-c3ccc(Cl)c(Cl)c3)o2)n(C)c1=O ; Cn1c(=O)c2c(nc(N3CCN(C(=O)CCOCCOCCOCCNc4cccc5c4C(=O)N(C4CCC(=O)NC4=O)C5=O)CC3)n2Cc2nnc(-c3ccc(Cl)c(Cl)c3)o2)n(C)c1=O ; Cn1c(=O)c2c(nc(N3CCN(C(=O)CCOCCOCCOCCOCCNc4cccc5c4C(=O)N(C4CCC(=O)NC4=O)C5=O)CC3)n2Cc2nnc(-c3ccc(Cl)c(Cl)c3)o2)n(C)c1=O]

Most preferably, the compound is:

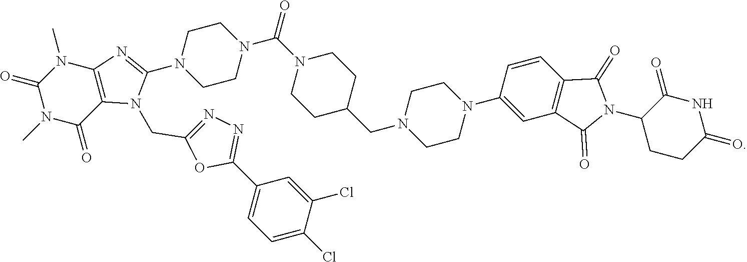 [SMILES: Cn1c(=O)c2c(nc(N3CCN(C(=O)N4CCC(CN5CCN(c6ccc7c(c6)C(=O)N(C6CCC(=O)NC6=O)C7=O)CC5)CC4)CC3)n2Cc2nnc(-c3ccc(Cl)c(Cl)c3)o2)n(C)c1=O]

### Synthesis of Intermediates

Also provided herein are processes for producing compounds of the invention, and intermediates in those processes.

A particularly preferred X group according to the invention is:

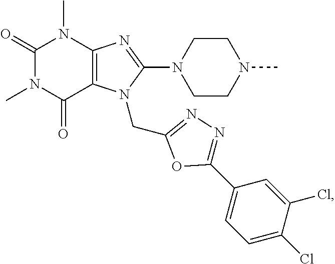

and this may advantageously joined to a linker containing an amide or thioamide group at the X-terminal end (that is, the end of linker L or LA proximal to the X group). The amide or thioamide group at the X-terminal end of the linker is generally positioned with the —C(═O)— or —C(═S)— group covalently bound to the X moiety.

However, this necessitates the formation of a heavily substituted urea or thiourea moiety:

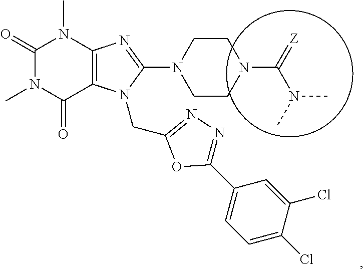

where Z is O or S, which can be difficult to manufacture. A suitable synthesis is shown below:

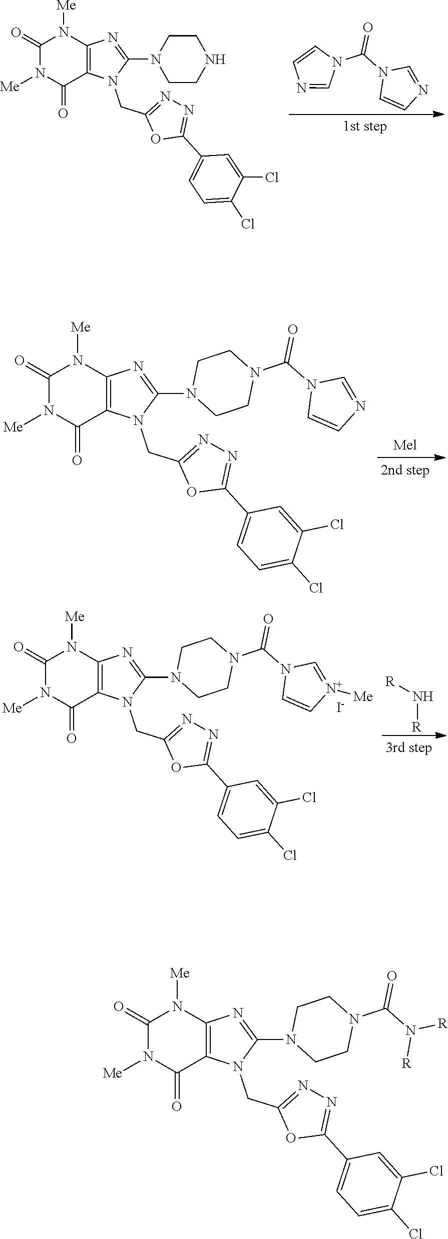

However, the second step was found to be challenging under usual conditions. It was surprisingly found that, performing this second step under exposure to microwaves increased yield significantly. In the absence of microwave exposure, reasonable conversion to product was found to take several days, even when heating to 60° C. However, under microwaves, at least 50% conversion of the starting material to product was achieved in as little as 15 minutes.

Accordingly, provided herein is an intermediate of formula (INT):

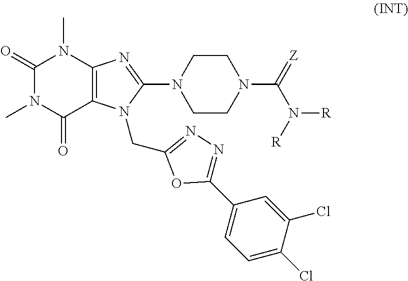

or a salt thereof,
wherein Z is O or S, and
each R is independently selected from alkyl, alkenyl, cycloalkyl, heterocycloalkyl, cycloalkenyl, heterocycloalkenyl, aryl or heteroaryl, or the two R groups are joined together to produce a heterocycloalkyl, heterocycloalkenyl or heteroaryl group.

Preferably Z is O.

Preferably, the two R groups are joined together to produce a heterocycloalkyl or heteroaryl group, particularly preferably a 5- to 7-membered heterocycloalkyl or 5- to 7-membered heteroaryl group. A 5- or 6-membered heteroaryl group is particularly preferred, especially a 5 or 6-membered heteroaryl group comprising two nitrogen atoms (including the nitrogen atom bound to C═Z).

R may be optionally substituted. Substituents may include halo, hydroxy (—OH), amino (—NH₂), alkyl, alkenyl, alkoxy, alkylthio, aminoalkyl, cycloalkyl, heterocycloalkyl, cycloalkenyl, heterocycloalkenyl, aryl or heteroaryl groups. Exemplary substituents are C₁-C₄ alkyl.

Particularly preferred intermediates are:

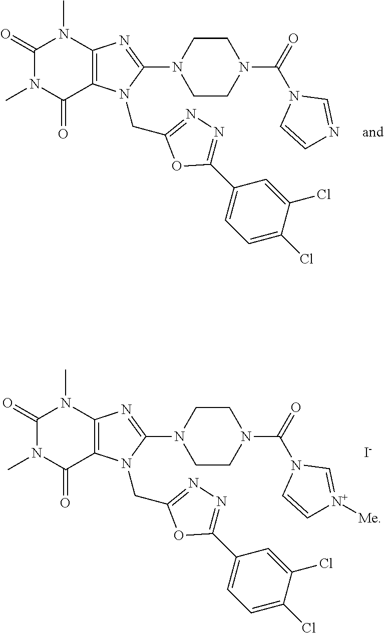 [SMILES: Cn1c(=O)c2c(nc(N3CCN(C(=O)n4ccnc4)CC3)n2Cc2nnc(-c3ccc(Cl)c(Cl)c3)o2)n(C)c1=O ; Cn1c(=O)c2c(nc(N3CCN(C(=O)n4cc[n+](C)c4)CC3)n2Cc2nnc(-c3ccc(Cl)c(Cl)c3)o2)n(C)c1=O ; [I-]]

Also provided is a process for producing the above intermediates of formula (INT). The process involves:
(i) treating an X-group precursor of formula (X) with a urea or thiourea compound of formula (INT-2):

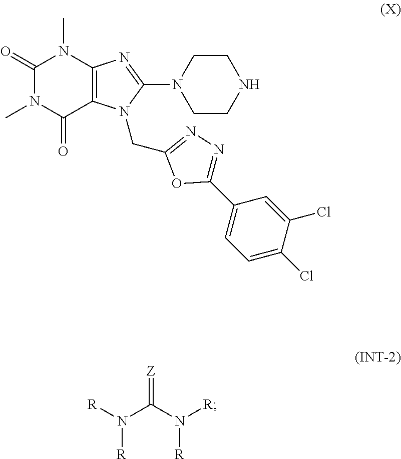

(ii) exposing the product of step (i) to an alkyliodide, under microwaves.

Preferably, the alkyliodide is methyliodide or ethyliodide, most preferably methyliodide.

Typically, the exposure to microwaves during step (ii) is performed for a period of from ten minutes to two hours, preferably from 15 minutes to 1 hour.

The solvent for step (i) is not particularly limited. Generally, a polar organic solvent is used. An exemplary solvent is dichloromethane.

In step (ii), a polar organic solvent is also typically used. A particular example is acetonitrile.

To form the degrader compounds described herein, (INT-2) is typically further reacted with one or more further reactants capable of providing the linker L or LA (unless it is present in the R groups provided), and the Y group. An exemplary reactant in this regard is:

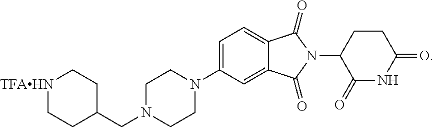

### Pharmaceutical Compositions

In one embodiment, the present invention provides a pharmaceutical composition comprising: (a) a compound of the present invention; and (b) a pharmaceutically acceptable carrier, diluent, and/or excipient. In one embodiment, a pharmaceutical composition of the present invention comprises compound of the present invention as well as a carrier, a stabilizer, an excipient, a diluent, a solubilizer, a surfactant, an emulsifier, and/or a preservative. In one embodiment, a pharmaceutical composition of the present invention is in solid or liquid form. In one embodiment, the pharmaceutical composition may be in the form of a powder, a tablet, a solution or an aerosol. In one embodiment, a pharmaceutical composition of the present invention is provided in a frozen form. In one embodiment, a pharmaceutical composition of the present invention is provided in lyophilized form.

A pharmaceutical composition of the present invention will usually be supplied as a sterile, pharmaceutical composition. In another embodiment, no such adjuvant is present in a pharmaceutical composition of the present invention. The present invention also provides a process for preparation of a pharmaceutical or medicament composition comprising adding and mixing compound of the present invention together with one or more of a pharmaceutically acceptable excipient, diluent or carrier.

Pharmaceutically acceptable carriers in therapeutic compositions may additionally contain liquids such as water, saline, glycerol and ethanol. Such carriers may be used, for example, so that the pharmaceutical compositions to be formulated as tablets, pills, dragées, capsules, liquids, gels, syrups, slurries and suspensions, for ingestion by the patient. The term “pharmaceutically acceptable excipient” as used herein typically refers to a pharmaceutically acceptable formulation carrier, solution or additive to enhance the desired characteristics of the compositions of the present invention. Excipients are well known in the art and include buffers (e.g., citrate buffer, phosphate buffer, acetate buffer, and bicarbonate buffer), amino acids, urea, alcohols, ascorbic acid, phospholipids, proteins (e.g., serum albumin), EDTA, sodium chloride, liposomes, mannitol, sorbitol, and glycerol. Solutions or suspensions can be encapsulated in liposomes or biodegradable microspheres. Suitable carriers may be large, slowly metabolised macromolecules such as proteins, polypeptides, liposomes, polysaccharides, polylactic acids, polyglycolic acids, polymeric amino acids, amino acid copolymers and inactive virus particles. Pharmaceutically acceptable salts can be used, for example mineral acid salts, such as hydrochlorides, hydrobromides, phosphates and sulphates, or salts of organic acids, such as acetates, propionates, malonates, and benzoates.

In certain embodiments, the pharmaceutical composition may contain formulation materials for the purpose of modifying, maintaining or preserving certain characteristics of the composition such as the pH, osmolarity, viscosity, clarity, color, isotonicity, odour, sterility, stability, rate of dissolution or release, adsorption or penetration. A thorough discussion of pharmaceutically acceptable carriers is available in Remington's Pharmaceutical Sciences (Mack Publishing Company, N.J. 1991). Additional pharmaceutical compositions include formulations involving the compound of the present invention in sustained or controlled delivery formulations. Techniques for formulating a variety of sustained- or controlled-delivery means are known to those skilled in the art. A compound of the present invention may also be entrapped in microcapsules prepared, for example, by coacervation techniques or by interfacial polymerization, in colloidal drug delivery systems, or in macroemulsions. Such techniques are also disclosed in Remington's Pharmaceutical Sciences.

A subject will be typically administered a therapeutically effective amount of a pharmaceutical composition and hence of a compound of the present invention. The term “therapeutically effective amount” typically refers to an amount of a therapeutic agent needed to treat, ameliorate or prevent a targeted disease or condition, or to exhibit a detectable therapeutic or preventative effect. In some embodiments it may be the amount needed to reduce or eliminate a side effect or effects of a second drug. The precise therapeutically effective amount for a human subject will depend upon the severity of the disease state, the general health of the subject, the age, weight and gender of the subject, diet, time and frequency of administration, drug combination(s), reaction sensitivities, and tolerance/response to therapy. This amount can be determined by routine experimentation and is within the judgement of the clinician. For example, a low dose may be used initially and then increased if needed to be based on the response seen. Generally, a therapeutically effective amount will be from 0.01 mg/kg to 50 mg/kg, for example 0.1 mg/kg to 20 mg/kg per day. Alternatively, the dose may be I to 500 mg per day, such as 10 to 100, 200, 300 or 400 mg per day. In one embodiment, the amount in a given dose is at least enough to bring about a particular function.

In one embodiment, a compound of the present invention may be given in combination with another treatment for the condition being treated. For example, a compound of the present invention may be provided simultaneously, sequentially, or separately with such a further agent. In another embodiment, a compound of the present invention may be provided in the same pharmaceutical composition as a second therapeutic agent. In one embodiment, a compound of the present invention may be given simultaneously, sequentially, or separately with methotrexate. In one embodiment, a composition is provided comprising a compound of the present and methotrexate. In another embodiment, a compound of the present invention may be given simultaneously, sequentially, or separately with 6-thioguanine (6-TG). In another embodiment, a composition is provided comprising a compound of the present invention and 6-TG. In embodiments where a compound of the present invention is provided for use in a method of treatment which comprises administering a second agent, the present invention further provides the second agent for use in a method of treatment which comprises administering a compound of the present invention.

In one preferred embodiment, the therapeutic agent of the invention, when in a pharmaceutical preparation, may be present in unit dose form. Suitable doses may be calculated for patients according to their weight, for example suitable doses may be in the range of 0.01 to 20 mg/kg, for example 0.1 to 20 mg/kg, for example 1 to 20 mg/kg, for example 10 to 20 mg/kg or for example I to 15 mg/kg, for example 10 to 15 mg/kg. To effectively treat conditions of use in the present invention in a human, suitable doses may be within the range of 0.001 to 10 mg, 0.01 to 1000 mg, for example 0.1 to 1000 mg, for example 0.1 to 500 mg, for example 500 mg, for example 0.1 to 100 mg, or 0.1 to 80 mg, or 0.1 to 60 mg, or 0.1 to 40 mg, or for example 1 to 100 mg, or 1 to 50 mg, of a dual targeting protein of this invention, which may be administered parenterally, for example subcutaneously, intravenously or intramuscularly. Such a dose may be, if necessary, repeated at appropriate time intervals selected as appropriate by a physician. A compound, of the present invention may be, for instance, lyophilized for storage and reconstituted in a suitable carrier prior to use. Lyophilization and reconstitution techniques can be employed.

The compounds and pharmaceutical compositions of this invention may be administered by any number of routes including, but not limited to, oral, intravenous, intramuscular, intra-arterial, intramedullary, intrathecal, intraventricular, transdermal, transcutaneous (for example, see WO 98/20734), subcutaneous, intraperitoneal, intranasal, enteral, topical, sublingual, intravaginal or rectal routes. A particularly preferred administration route for a composition of the present invention is topically. A further preferred administration route for a composition of the present invention is orally. Hyposprays may also be used to administer the pharmaceutical compositions of the invention. Direct delivery of the compositions will generally be accomplished by injection, subcutaneously, intraperitoneally, intravenously or intramuscularly, or delivered to the interstitial space of a tissue. In one preferred embodiment, administration is via intravenous administration. In another preferred embodiment, administration is via subcutaneous administration, for example via subcutaneous injection. The compositions can also be administered into a specific tissue of interest. In some embodiments, administration is via site-specific or targeted local delivery techniques. Examples of site-specific or targeted local delivery techniques include various implantable depot sources of the compound or local delivery catheters, such as infusion catheters, indwelling catheters, or needle catheters, synthetic grafts, adventitial wraps, shunts and stents or other implantable devices, site specific carriers, direct injection, or direct application.

Dosage treatment may be a single dose schedule or a multiple dose schedule. Where the product is for injection or infusion, it may take the form of a suspension, solution or emulsion in an oily or aqueous vehicle and it may contain formulary agents, such as suspending, preservative, stabilising and/or dispersing agents. Alternatively, the pharmaceutical may be in dry form, for reconstitution before use with an appropriate sterile liquid. In one embodiment, a pharmaceutical composition comprising a compound of the present invention is provided in lyophilised form. If a composition is to be administered by a route using the gastrointestinal tract, the composition will typically need to contain agents which protect the compound from degradation but which release the compound once it has been absorbed from the gastrointestinal tract. In another embodiment, a nebulisable formulation according to the present invention may be provided, for example, as single dose units (e.g., sealed plastic containers or vials) packed in foil envelopes. Each vial contains a unit dose in a volume, e.g., 2 ml, of solvent/solution buffer.

A pharmaceutical composition of the present invention may be provided in a receptacle that provides means for administration to a subject. In one embodiment, a pharmaceutical composition of the present invention may be provided in a prefilled syringe. The present invention therefore provides such a loaded syringe. It also provides an auto-injector loaded with a pharmaceutical composition of the present invention.

In one embodiment the formulation is provided as a formulation for topical administrations including inhalation. Suitable inhalable preparations include inhalable powders, metering aerosols containing propellant gases or inhalable solutions free from propellant gases. Inhalable powders according to the invention containing the active substance may consist solely of the abovementioned active substances or of a mixture of the abovementioned active substances with physiologically acceptable excipient. These inhalable powders may include monosaccharides (e.g., glucose or arabinose), disaccharides (e.g., lactose, saccharose, maltose), oligo- and polysaccharides (e.g., dextranes), polyalcohols (e.g., sorbitol, mannitol, xylitol), salts (e.g., sodium chloride, calcium carbonate) or mixtures of these with one another. Mono- or disaccharides are suitably used, the use of lactose or glucose, particularly but not exclusively in the form of their hydrates.

Particles for deposition in the lung require a particle size less than 10 microns, such as 1-9 microns for example from 1 to 5 μm. The particle size of the active ingredient such as the compound of primary importance. The propellant gases which can be used to prepare the inhalable aerosols are known in the art. Suitable propellant gases are selected from among hydrocarbons such as n-propane, n-butane or isobutane and halohydrocarbons such as chlorinated and/or fluorinated derivatives of methane, ethane, propane, butane, cyclopropane or cyclobutane. The above mentioned propellent gases may be used on their own or in mixtures thereof. Particularly suitable propellent gases are halogenated alkane derivatives selected from among TG 11, TG 12, TG 134a and TG227. Of the above-mentioned halogenated hydrocarbons, TG134a (1,1,1,2-tetrafluoroethane) and TG227 (1,1,1,2,3,3,3-heptafluoropropane) and mixtures thereof are particularly suitable. The propellent-gas-containing inhalable aerosols may also contain other ingredients such as cosolvents, stabilisers, surface-active agents (surfactants), antioxidants, lubricants and means for adjusting the pH. All these ingredients are known in the art. The propellant-gas-containing inhalable aerosols according to the invention may contain up to 5% by weight of active substance. Aerosols according to the invention contain, for example, 0.002 to 5% by weight, 0.01 to 3% by weight, 0.015 to 2% by weight, 0.1 to 2% by weight, 0.5 to 2% by weight or 0.5 to 1% by weight of active ingredient.

Alternatively topical administrations to the lung may also be by administration of a liquid solution or suspension formulation, for example employing a device such as a nebulizer, for example, a nebulizer connected to a compressor (e.g., the Pari LC-Jet Plus(R) nebulizer connected to a Pari Master(R) compressor manufactured by Pari Respiratory Equipment, Inc., Richmond, Va.).

Nebulisable formulation according to the present invention may be provided, for example, as single dose units (e.g., sealed plastic containers or vials) packed in foil envelopes. Each vial contains a unit dose in a volume, e.g., 2 mL, of solvent/solution buffer. The present invention also provides a syringe loaded with a composition comprising a compound of the invention. In one embodiment, a pre-filled syringe loaded with a unit dose of a compound is provided. In another embodiment, an autoinjector loaded with a compound of the invention is provided. In a further embodiment, an IV bag loaded with a pharmaceutical composition of the invention is provided.

Once formulated, the compositions of the invention can be administered directly to the subject. By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on. In a preferred embodiment, the subject to be treated is a mammal. The subjects to be treated can be animals. However, in one or more embodiments the compositions are adapted for administration to humans. In a particularly preferred embodiment, the subject is human.

### Kits

The present invention also extends to a kit comprising a compound of the invention, optionally with instructions for administration. In another embodiment, a kit containing single-chambered or multi-chambered pre-filled syringes is provided which is pre-filled with a pharmaceutical composition of the invention. The invention also provides a kit for a single-dose administration unit which comprises a pharmaceutical composition of the invention. In another embodiment, the kit comprises packaging.

### Pathological Conditions, Medical, and Diagnostic Uses

In another embodiment, a compound of the present invention is provided for use in a method of treatment of the human or animal body. Also provided is a compound of the present invention for use as a medicament. Please note that, in the various therapeutic uses set out herein where reference is made to a compound of the present invention, a pharmaceutical composition comprising it may be also employed and vice versa unless stated otherwise. The present invention also provides the various methods of treatment set out herein employing a compound of the present invention. A compound of the present invention may also be used in diagnosis, including in both in vivo diagnosis and also in vitro diagnosis, for example such diagnosis performed on a sample from a subject.

As discussed further below, a compound of the present invention may be employed to treat a condition. As used herein, the terms “treat” or “treatment” refer to both therapeutic treatment and prophylactic or preventative measures, wherein the object is to prevent or slow down (lessen) an undesired physiological change or disorder. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. Treatment can also involve the reduction or elimination of the side-effect of a second drug.

As has been shown in the accompanying Examples, effective “knock-out” of the NUDT5 protein using compounds as described herein leads to reduced adenosine toxicity in cells and increased proliferation. Adenosine is produced in the complex folate pathway, and an accumulation of adenosine, which is toxic in cells, can occur if that pathway is impaired. Degradation of NUDT5 using the compounds described herein has been shown to modulate the presence of adenosine, and consequently the compounds have been show to “rescue” adenosine toxicity in cells. Accordingly, in an embodiment, a compound of the invention is provided for use in the treatment of adenosine toxicity in a patient. In another embodiment, a compound of the invention is provided for use in the modulation of adenosine response in a patient.

Adenosine toxicity may be observed in patients having an impaired folate pathway, such as an abnormal folate metabolism and other such deficiencies. Accordingly, in a further embodiment, a compound of the invention is provided to treat a folate related disorder. For instance, a compound of the invention may be used to treat a patient less able to metabolise folate, or having a deficiency in the folate pathway. Such patients typically have a mutant MTHFD1 gene, and so the compound may be used to treat a patient with a genetic disorder related to MTHFD1.

A further function of NUDT5 in cells is its involvement in the complex mechanism of nuclear ATP synthesis (described in, for instance, EP2930238, which is incorporated herein by reference). It has previously been found that degradation of poly-ADP-ribose (PAR) in cells is a key part of ATP synthesis. As cancer cells have a particularly high requirement for nuclear ATP, blocking this aspect of the ATP synthesis pathway has been found to affect the proliferation of cancer cells. Previously, therefore, inhibition of NUDT5 (which is involved in the degradation of PAR) has been suggested as a mechanism for inhibiting the proliferation of cancer cells. Degradation of NUDT5 is expected to have a corresponding or even more significant effect on the proliferation of cancer cells.

Accordingly, in one preferred embodiment, a compound of the present invention may be used to treat cancer. In one preferred embodiment, the cancer is a leukaemia. In one preferred embodiment, the cancer is acute myelogenous leukaemia (AML). In another preferred embodiment, the cancer is acute lymphoblastic leukaemia (ALL). In one embodiment, the cancer is chronic myelogenous leukaemia (CML). In a further preferred embodiment, the cancer is breast cancer. In one embodiment, the cancer is head cancer, neck cancer, or a sarcoma. In one embodiment, the cancer is gestational choriocarcinoma (GC) or gestational trophoblastic disease (GT). In a further embodiment, the cancer is lung cancer. In one embodiment, the cancer is a paediatric cancer. In one embodiment, the cancer is a solid tumour. In another embodiment, the cancer is not a solid-tumour, for example it is a leukaemia.

The present findings concerning degradation of NUDT5 also suggest a particularly advantageous role for the compounds of the invention in the treatment of diseases such as cancer in combination with one or more other drugs. The accompanying Examples demonstrate that NUDT5 has a key role in the purine de novo synthesis. One effect of adenosine toxicity is to repress purine de novo synthesis and related compounds. However, the ability of the compounds of the invention to modulate that toxicity is also shown herein to reduce the accompanying repression, leading to increased purine de novo synthesis, and lower total purine levels overall. That is, the metabolism of purine was found to be restored by degradation of NUDT5. This suggests a significant combinatorial effect for the NUDT5 degraders of the invention in combination with a drug which induce adenosine toxicity in cells, and/or which is involved in the purine pathway. Combining such drugs with degraders of the invention may reduce the toxicity of the drugs in healthy cells, leading to more efficacious treatment (for instance, or cancer or inflammatory disorders).

One suitable drug expected to act advantageously in combination with a compound of the invention a purine analogue, such as 6-thioguanine. Another drug expected to act advantageously in combination with a compound of the invention is a compound used to purine de novo synthesis, such as methotrexate (as described in US 2003/166007, incorporated herein by reference).

Thus, also provided herein is a compound of the invention for use in treatment of cancer together with another anti-cancer agent, wherein the anti-cancer agent is a purine analogue or an agent which disrupts purine de novo synthesis.

In one embodiment, the cancer is one that is also being treated with methotrexate. In another embodiment, the cancer is one that is being treated with 6-thioguanine. In one embodiment, a compound of the present invention is given as part of a combination therapy for treating cancer. In one embodiment, a compound of the present invention is provided for use in method of chemotherapy.

In one embodiment, the cancer may be one selected from acute myeloid leukemia, breast carcinoma, colorectal adenocarcinoma, diffuse large B-cell lymphoma, endometrial adenocarcinoma, follicular lymphoma, lung adenocarcinoma, melanoma, ovarian adenocarcinoma, pancreatic adenocarcinoma, pleural mesothelioma, B-cell acute lymphoblastic Leukemia, T-cell Acute Lymphoblastic Leukemia, prostate carcinoma and renal cell carcinoma.

In a further preferred embodiment the cancer is a hormone-dependent cancer. A hormone-dependent cancer refers to a cancer that has hormonal sensitivity. Examples of said cancers are, without limitation, breast, endometrium, ovary, prostate, testis, thyroid and osteosarcoma cancer. In a preferred embodiment the cancer is a steroid-dependent cancer, more preferred estrogen and progestin cancer. In a more preferred embodiment the progestin dependent cancer is a progestin-dependent breast cancer. In another more preferred embodiment, the cancer is an androgen dependent cancer, more preferably androgen-dependent prostate cancer.

In one embodiment, the cancer is selected from a carcinoma, lymphoma and sarcoma.

A further embodiment which the invention may be employed to treat is cardiovascular disease. In another embodiment, the invention may be employed to treat liver fibrosis.

In a further embodiment, a compound of the invention may be used to treat a folate related disorder.

In one embodiment, the condition to be treated may be an inflammatory disorder. In another embodiment, the condition may be an autoimmune disorder. In a preferred embodiment, the condition to be treated may be arthritis. In a particularly preferred embodiment, it may be rheumatoid arthritis. In another embodiment, the condition may be psoriasis. In another embodiment, the condition may be an inflammatory bowel disease (IBD). In one embodiment, the condition may be Crohn's Disorder. In one embodiment, the subject may be one who is also being treated with methotrexate.

In one embodiment, the condition to be treated is a metabolic disorder. In a preferred embodiment, it is Methylenetetrahydrofolate Reductase (MTHFR) Deficiency. In one embodiment, the condition is hyperhomocysteinemia.

In one embodiment, a compound of the present invention is given to reduce the side-effects or toxicity of a second drug. (In such embodiments, the compound of the present is for use in treating a subject also being treated with the said second drug). It may be that the administration of a compound of the present invention reduces the side-effects of administration of the second drug. For example, it may be that administration of the compound reduces the severity or incidence of a side-effect. In one embodiment, a compound of the present invention is given to reduce the side-effects of a cancer therapy. In one embodiment, it is given to reduce a side-effect or effects of chemotherapy. In another embodiment, a compound of the present invention may be used to reduce the toxicity and/or the side-effects of an immunosuppressive agent.

The second drug may be being administered to treat an inflammatory disorder, autoimmune disorder, cancer or metabolic disorder in a subject. Thus, on one embodiment, the second drug is an anti-cancer drug. In another embodiment, the second drug is an anti-arthritis drug. The second drug may be methotrexate or 6-thioguanine (6-TG).

In one embodiment, a compound of the present invention is given to a patient who is also being treated with methotrexate to reduce the side-effects of the methotrexate. In one such embodiment, the condition being treated is a cancer. In another such embodiment, the condition being treated is rheumatoid arthritis. In one embodiment, a compound of the present invention is given to a subject being treated with 6-thioguanine to reduce a side-effect or effects of the drug. In one embodiment, a compound of the present invention is given to such a subject who is being treated with 6-thioguanine to treat cancer.

In another embodiment, a compound of the invention may be used to inhibit the interaction between NUDT-5 and phosphoribosyl pyrophosphate amidotransferase (PPAT). The present invention also provides compounds in general able to inhibit the interaction between NUDT-5 and phosphoribosyl pyrophosphate amidotransferase (PPAT) for use in treating any of the conditions referred to herein.

### SYNTHESIS EXAMPLES

Commercial reagents and solvents were purchased from commercial suppliers and used without further purification. All reactions involving moisture sensitive reagents were carried out under a nitrogen atmosphere using standard vacuum line techniques and dry solvents.

An Elga DV 25 system was used for deionising water.

Thin layer chromatography was performed on aluminium plates coated with 60 F₂₅₄ silica gel. Plates were visualised using UV light (254 nm).

Flash column chromatography was performed on a Biotage Isolera one flash column chromatography platform.

¹H and ¹³C NMR spectra were obtained using Bruker NMR spectrometers (400 MHz). The proton and carbon chemical shift values are reported in parts per million (ppm, δ scale) downfield from tetramethylsilane (TMS) and the indicated solvent. NMR spectra were processed and analyzed using MestReNova software. Spin multiplicities are given as s (singlet), d (doublet), t (triplet), q (quartet), dd (doublet of doublets), m (multiplet) and b (broad), coupling constants J are given in hertz (Hz), and signal area integration in natural numbers.

LCMS was performed with a Kinetex 5μ EVO C18 100A 100×3.0 mm column on a Waters SFO and 515 HPLC pump and Waters Binary Gradient 2545 device using linear gradient of solvent A (93% H₂O, 5% acetonitrile and 2% of 0.5 M ammonium acetate pH 6.0) and solvent B (18% H₂O, 80% acetonitrile and 2% ammonium acetate pH 6.0), eluting at a flow rate of 2 mL/min: 5% B for 0.35 min, 5% B to 95% B for 1 min, 95% to 5% B for 0.1 min and 5% B for 0.8 min. LCMS was used as a measure of compound purity using either UV absorbance (Waters UV/visible Detector 2489), ELSD signal (Waters ELS Detector 2424) or ESI+TIC (SQ Detector 2). Preparative HPLC was performed on the same system with a Kinetex 5u EVO C18 100A 150×21.2 mm column using linear gradient of solvent A over 20 min from 85% to 10% eluting at a flow rate of 20 mL/min. LCMS was acquired using Waters FractionLynx software and processed using MestReNova softwar. HRMS was acquired with Agilent 6530 RapidFire QTOF mass spectrometer in 384-well polypropylene plates (Greiner, 781280) using an assay buffer consisting of 10 mM ammonium formate pH 7.5. The plate was transferred to a RapidFire RF360 high-throughput sampling robot. Samples were aspirated under vacuum and loaded onto a C4 solid-phase extraction (SPE) cartridge equilibrated and washed for 4.5 s with 0.1% formic acid in LCMS grade water to remove non-volatile buffer components. After aqueous wash, analytes of interest were eluted from the C4 SPE onto an Agilent 6530 accurate mass Q-TOF in an organic elution step, 85% acetonitrile and 0.1% formic acid in LCMS grade water. Ion data for the compounds were extracted and compound formulas were generated using MassHunter Qualitative Analysis B.06.00 (Agilent).

### Synthesis of Exemplary Compound and Negative Control

Procedures for Synthesis of dNUDT5 (Exemplary Compound)

### Synthesis of 8-(4-(1H-imidazole-1-carbonyl)piperazin-1-yl)-7-((5-(3,4-dichlorophenyl)-1,3,4-oxadiazol-2-yl)methyl)-1,3-dimethyl-3,7-dihydro-1H-purine-2,6-dione

Adapted from reference [1].

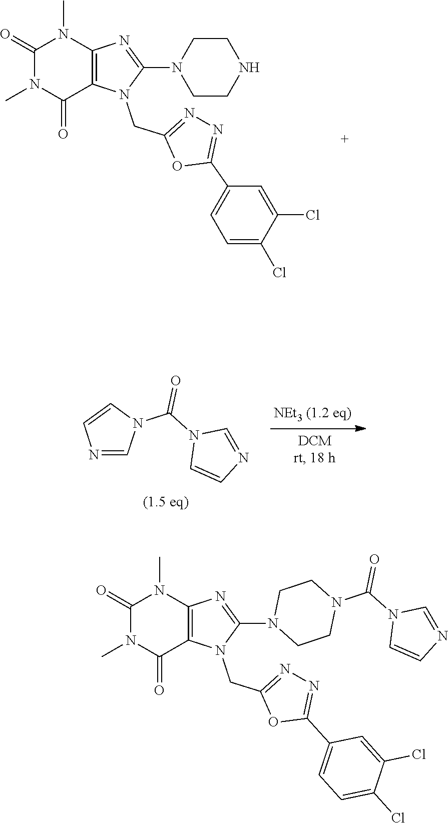 [SMILES: Cn1c(=O)c2c(nc(N3CCNCC3)n2Cc2nnc(-c3ccc(Cl)c(Cl)c3)o2)n(C)c1=O ; O=C(n1ccnc1)n1ccnc1 ; Cn1c(=O)c2c(nc(N3CCN(C(=O)n4ccnc4)CC3)n2Cc2nnc(-c3ccc(Cl)c(Cl)c3)o2)n(C)c1=O]

Under nitrogen, 1,1′-carbonyldiimidazole (88.6 mg, 0.546 mmol, 1.5 eq) was added at −5° C. to a mixture of TH5427 (179 mg, 0.364 mmol, 1 eq) and triethylamine (61 μL, 0.437 mmol, 1.2 eq) in DCM (14.3 mL). The mixture was then stirred overnight at room temperature. After concentration under reduced pressure, methanol was added and the resulting solid was collected by filtration to afford the expected product as white solid (213 mg, 0.364 mmol, quantitative).

¹H NMR (400 MHz, CDCl3) δ 8.12 (d, 1H, J=2.0 Hz), 8.03 (bs, 1H), 7.87 (dd, 1H, J=2.0 Hz J=8.4 Hz), 7.61 (d, 1H, J=8.4 Hz), 7.24 (bs, 1H), 7.16 (bs, 1H), 5.77 (s, 2H), 3.79-3.77 (m, 4H), 3.56 (s, 3H), 3.41-3.40 (m, 4H), 3.39 (s, 3H).

¹³C NMR (101 MHz, CDCl3) δ 164.3, 161.9, 155.2, 155.1, 151.6, 150.9, 147.6, 137.1, 137.0, 134.0, 131.6, 129.8, 128.9, 126.2, 123.0, 118.1, 105.1, 50.5, 45.9 (2C), 40.0 (2C), 30.0, 28.0.

HPLC-MS t_{R}=1.458 min (97.2%), ESI+m/z 585.533; 587.153 [M+H]⁺

### Synthesis of 7-((5-(3,4-dichlorophenyl)-1,3,4-oxadiazol-2-yl)methyl)-8-(4-(4-((4-(2-(2,6-dioxopiperidin-3-yl)-1,3-dioxoisoindolin-5-yl)piperazin-1-yl)methyl)piperidine-1-carbonyl)piperazin-l-yl)-1,3-dimethyl-3,7-dihydro-1H-purine-2,6-dione

1^{st} Step Adapted from Reference [2].

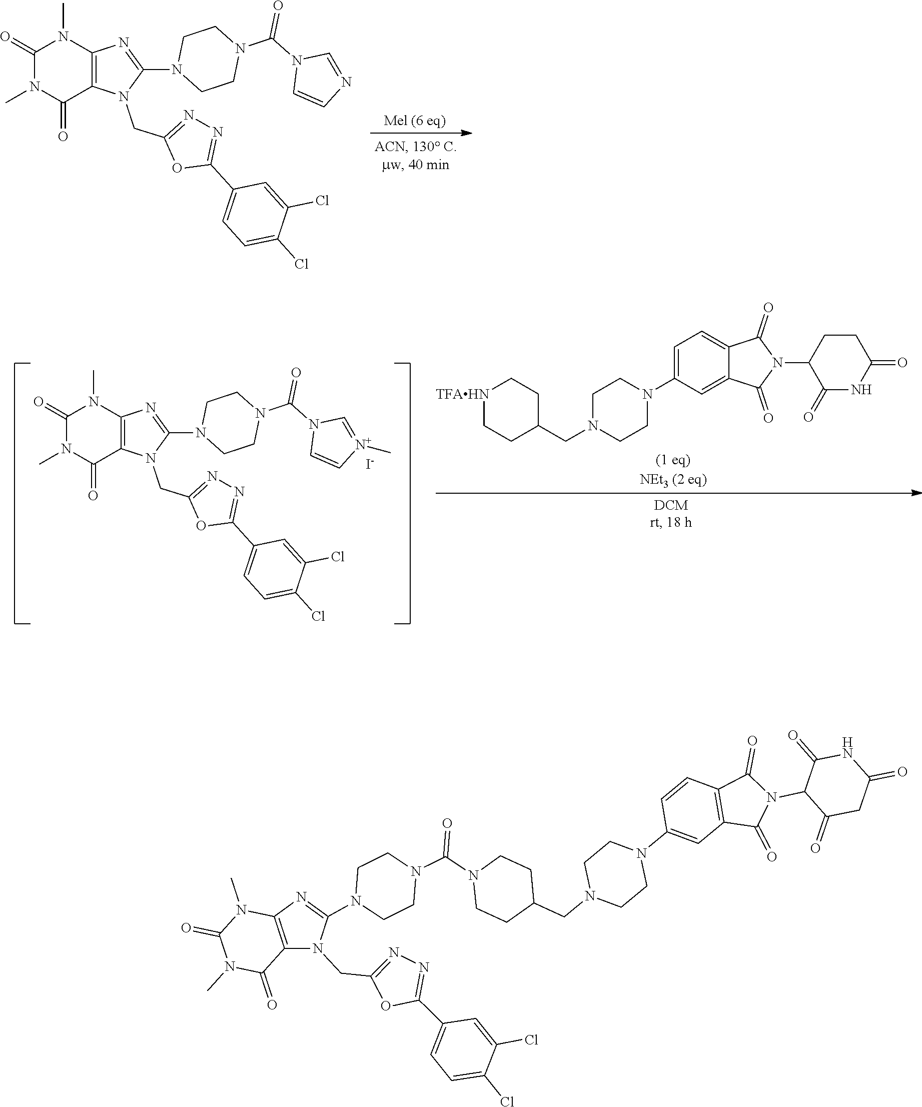

Under N₂, methyl iodide (16 μL, 0.256 mmol, 6 eq) was added to a mixture of 8-(4-(1H-imidazole-1-carbonyl)piperazin-1-yl)-7-((5-(3,4-dichlorophenyl)-1,3,4-oxadiazol-2-yl)methyl)-1,3-dimethyl-3,7-dihydro-1H-purine-2,6-dione (25 mg, 0.043 mmol, 1 eq) in acetonitrile (1.7 mL). The mixture was stirred for 40 min at 130° C. under microwave heating (the reaction was checked by LCMS—100% conversion). After concentration under reduced pressure, the crude was dissolved in DCM (2 mL). Under N₂, triethylamine (12 μL, 0.086 mmol, 2 eq) and 2-(2,6-dioxopiperidin-3-yl)-5-(4-((1-(2,2,2-trifluoroacetyl)-1λ4-piperidin-4-yl)methyl)piperazin-1-yl)isoindoline-1,3-dione, trifluoroacetic salt (23.8 mg, 0.043 mmol, 1 eq) were added.

The mixture was stirred overnight at room temperature. After concentration under reduced pressure, the crude was purified by HPLC to afford the expected product as yellow solid (6.9 mg, 0.007 mmol, 17% over 2 steps).

¹H NMR (400 MHz, CDCl₃) δ 8.11 (d, J=2.0 Hz, 1H), 8.06 (s, 1H), 7.86 (dd, J=2.0 Hz, J=8.4 Hz, 1H), 7.67 (d, J=8.4 Hz, 1H), 7.60 (d, J=8.4 Hz, 1H), 7.28 (d, J=2.2 Hz, 1H), 7.05 (dd, J=2.0 Hz, J=8.5 Hz, 1H), 5.66 (s, 2H), 4.96-4.91 (m, 1H), 3.75-3.72 (m, 2H), 3.55 (s, 3H), 3.43-3.40 (m, 4H), 3.37 (s, 3H), 3.35-3.33 (m, 3H), 3.30-3.28 (m, 4H), 2.91-2.81 (m, 2H), 2.80-2.69 (m, 3H), 2.60-2.55 (m, 3H), 2.27-2.26 (m, 2H), 2.17-2.10 (m, 1H), 1.81-1.78 (m, 2H), 1.74-1.69 (m, 2H), 1.21-1.12 (m, 2H)

¹³C NMR (101 MHz, CDCl₃) δ 171.0, 168.3, 168.0, 167.3, 164.1, 164.0, 162.1, 156.3, 155.6, 155.0, 151.7, 147.9, 136.9, 134.4, 134.0, 131.5, 128.9, 126.2, 125.5, 123.1, 119.7, 118.0, 108.8, 104.9, 64.4, 53.2 (2C), 50.5 (2C), 49.3, 47.6 (2C), 47.1 (2C), 46.5 (2C), 40.2, 33.9, 31.6, 30.8 (2C), 30.0, 28.0, 22.9.

HPLC-MS t_{R}=1.558 min (96.9%), ESI+m/z 957.9246; 959.9651 [M+H]⁺

HRMS was calculated for C₄₄H₄₈Cl₂N₁₃O₈: 956.3126, found: 956.3120.

Procedures for Synthesis of dNUDT5nc (Negative Control)

### Synthesis of 2-(1-methyl-2,6-dioxopiperidin-3-yl)-5-(piperazin-1-yl)isoindoline-1.3-dione (trifluoroacetic salt)

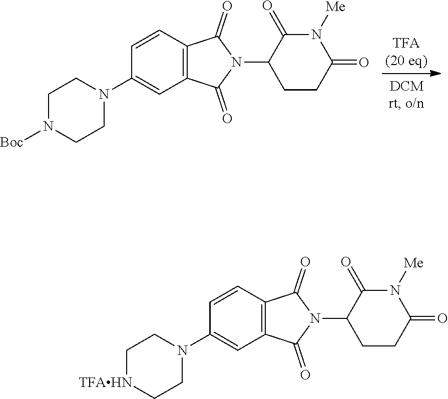

Trifluoroacetic acid (429 μL, 5.564 mmol, 20 eq) was added dropwise to a solution of tert-butyl 4-(2-(1-methyl-2,6-dioxopiperidin-3-yl)-1,3-dioxoisoindolin-5-yl)piperazine-1-carboxylate (127 mg, 0.278 mmol, 1 eq) in DCM (4 mL) at 0° C. The mixture was stirred for 3 h at room temperature. Water was added and the aqueous layer was recovered and concentrated under reduced pressure to afford the expected product as a yellow oil (99.5 mg, 0.212 mmol, 76%).

¹H NMR (400 MHz, MeOD) δ 7.74 (d, J=8.5 Hz, 1H), 7.44 (d, J=2.3 Hz, 1H), 7.32 (dd, J=2.3 Hz, J=8.5 Hz, 1H), 5.11 (dd, J=5.5 Hz, J=13.0 Hz, 1H), 3.71-3.69 (m, 4H), 3.41-3.38 (m, 4H), 3.31 (s, 3H), 2.91-2.87 (m, 2H), 2.75-2.68 (m, 1H), 2.13-2.06 (m, 1H).

¹³C NMR (101 MHz, MeOD) δ 173.6, 171.4, 169.0, 168.7, 156.2, 135.5, 126.1, 120.6, 110.5, 51.2, 49.0, 46.0 (2C), 44.3 (2C), 32.5, 27.3, 22.9 (TFA not detected).

HPLC-MS t_{R}=1.083 min (100%), ESI+m/z 357.4402 [M+H]⁺

### Synthesis of tert-butyl 4-((4-(2-(1-methyl-2,6-dioxopiperidin-3-yl)-1,3-dioxoisoindolin-5-yl)piperazin-1-yl)methyl)piperidine-1-carboxylate

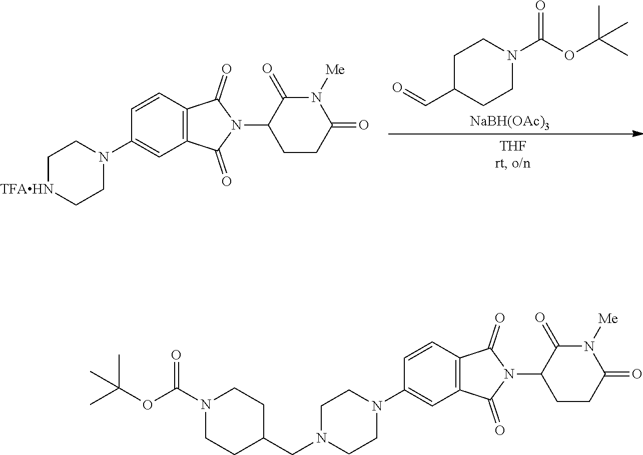

Sodium triacetoxyborohydride (67 mg, 0.316 mmol, 1.5 eq) was added to a mixture of 2-(1-methyl-2,6-dioxopiperidin-3-yl)-5-(piperazin-1-yl)isoindoline-1,3-dione, trifluoroacetic salt (99 mg, 0.210 mmol, 1 eq) and tert-butyl 4-formylpiperidine-1-carboxylate (54 mg, 0.253 mmol, 1.2 eq) in THE (4.6 mL). The mixture was stirred overnight at room temperature. After concentration under reduced pressure, the crude was purified by column chromatography (Si-35, DCM/MeOH gradient from 100% DCM to 95%) to afford the expected product as yellow solid (112.3 mg, 0.203 mmol, 96%).

¹H NMR (400 MHz, CDCl3) δ 7.65 (d, J=8.6 Hz, 1H), 7.24 (d, J=2.3 Hz, 1H), 7.02 (dd, J=2.3 Hz, J=8.6 Hz, 1H), 4.94-4.90 (m, 1H), 4.07 (bs, 2H), 3.41 (bs, 4H), 3.16 (s, 3H), 2.98-2.89 (m, 1H), 2.81-2.65 (m, 4H), 2.58-2.56 (m, 4H), 2.24 (bs, 2H), 2.08-2.04 (m, 1H), 1.75-1.67 (m, 3H), 1.43 (s, 9H), 1.13-1.03 (m, 2H).

¹³C NMR (101 MHz, CDCl₃) δ 171.3, 169.1, 168.1, 167.4, 155.4, 154.9, 134.3, 134.3, 125.3, 117.9, 108.6, 79.3, 64.3, 53.0 (4C), 49.9, 47.4 (2C), 33.5, 32.0 (2C), 30.7, 28.5, 27.2, 22.1 (C═O Boc not detected).

HPLC-MS t^{R}=1.767 min (97.3%), ESI+m/z 554.8069 [M+H]⁺

### Synthesis of 2-(1-methyl-2,6-dioxopiperidin-3-yl)-5-(4-(piperidin-4-ylmethyl)piperazin-1-yl)isoindoline-1,3-dione (trifluoroacetic salt)

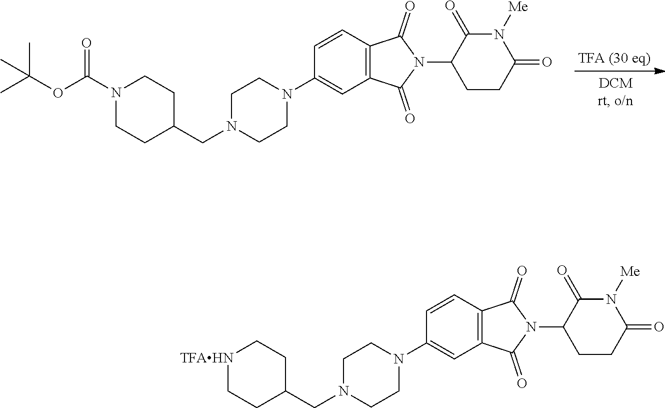

Trifluoroacetic acid (468 μL, 6.069 mmol, 30 eq) was added dropwise to a solution of tert-butyl 4-((4-(2-(1-methyl-2,6-dioxopiperidin-3-yl)-1,3-dioxoisoindolin-5-yl)piperazin-1-yl)methyl)piperidine-1-carboxylate (835 mg, 1.547 mmol, 1 eq) in DCM (10 mL) at 0° C. The mixture was stirred for 3 h at room temperature. Water was added and the aqueous layer was recovered and concentrated under reduced pressure to afford the expected product as a yellow solid (863 mg, 1.54 mmol, quantitative).

¹H NMR (400 MHz, MeOD) δ 7.77 (d, J=8.5 Hz, 1H), 7.48 (d, J=2.3 Hz, 1H), 7.35 (dd, J=2.3 Hz, J=8.5 Hz, 1H), 5.15-5.10 (m, 1H), 3.78 (bs, 3H), 3.48-3.45 (m, 6H), 3-18-3.16 (m, 2H), 3.14 (s, 3H), 3.09-3.02 (m, 2H), 2.91-2.87 (m, 2H), 2.75-2.64 (m, 1H), 2.32-2.27 (m, 1H), 2.12-2.08 (m, 3H), 1.61-1.50 (m, 2H) (TFA not detected).

¹³C NMR (101 MHz, MeOD) δ 173.6, 171.4, 169.0, 168.7, 155.9, 135.6, 126.1, 122.9, 120.5, 110.4, 62.3, 53.1 (2C), 51.2, 46.1 (2C), 44.4 (2C), 32.5, 30.3, 27.8 (2C), 27.3, 23.0 (TFA not detected).

### Synthesis of 7-((5-(3,4-dichlorophenyl)-1,3,4-oxadiazol-2-yl)methyl)-1,3-dimethyl-8-(4-(4-((4-(2-(1-methyl-2,6-dioxopiperidin-3-yl)-1,3-dioxoisoindolin-5-yl)piperazin-1-yl)methyl)piperidine-1-carbonyl)piperazin-1-yl)-3,7-dihydro-1H-purine-2,6-dione

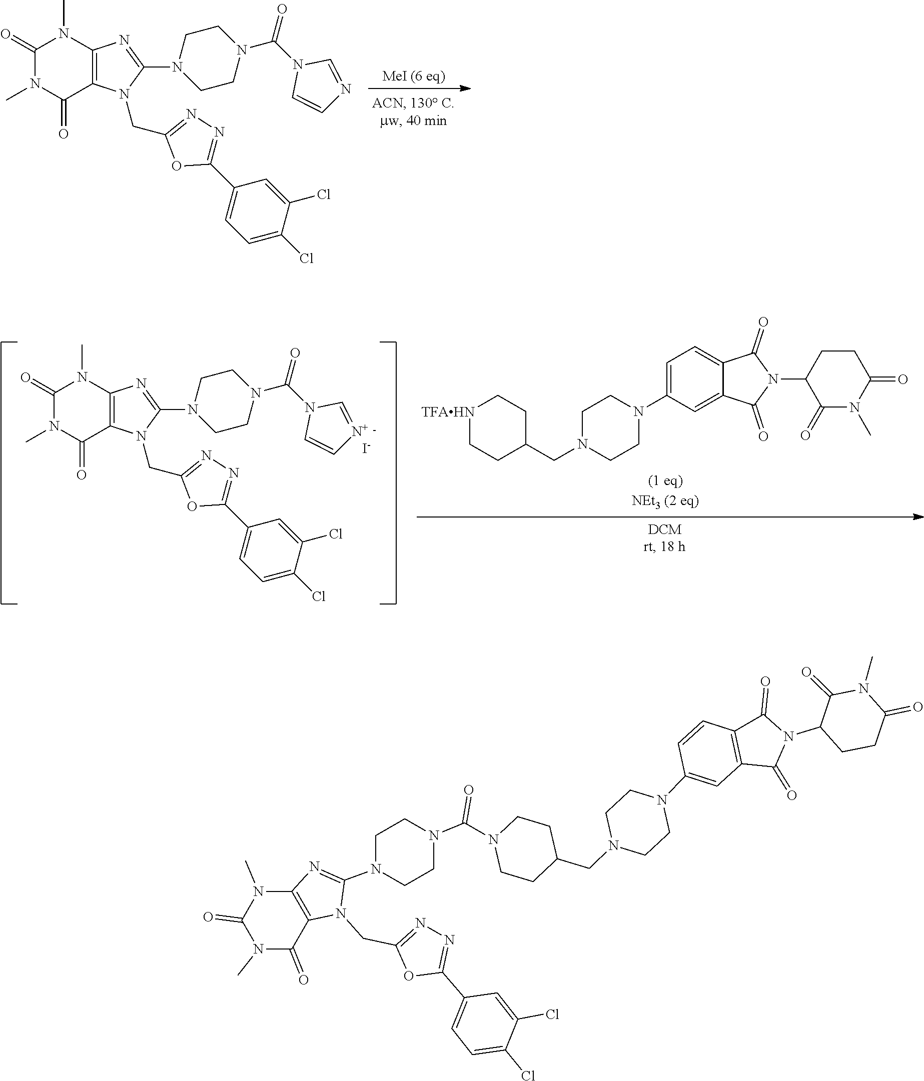

Under N₂, methyl iodide (16 μL, 0.256 mmol, 6 eq) was added to a mixture of 8-(4-(1H-imidazole-1-carbonyl)piperazin-1-yl)-7-((5-(3,4-dichlorophenyl)-1,3,4-oxadiazol-2-yl)methyl)-1,3-dimethyl-3,7-dihydro-1H-purine-2,6-dione (25 mg, 0.043 mmol, 1 eq) in acetonitrile (1.7 mL). The mixture was stirred for 40 min at 130° C. under microwave heating (the reaction was checked by LCMS 95% conversion). After concentration under reduced pressure, the crude was dissolved in DCM (1.8 mL). Under N₂, were added triethylamine (12 μL, 0.086 mmol, 2 eq) and 2-(1-methyl-2,6-dioxopiperidin-3-yl)-5-(4-(piperidin-4-ylmethyl)piperazin-1-yl)isoindoline-1,3-dione, trifluoroacetic salt (24.4 mg, 0.043 mmol, 1 eq). The mixture was stirred overnight at room temperature. After concentration under reduced pressure, the crude was purified by HPLC to afford the expected product as yellow solid (5.9 mg, 0.006 mmol, 14% over 2 steps).

¹H NMR (400 MHz, CDCl3) δ 8.12 (d, J=2.0 Hz, 1H), 7.87 (dd, J=2.0 Hz, J=8.4 Hz, 1H), 7.70 (d, J=8.4 Hz, 1H), 7.60 (d, J=8.4 Hz, 1H), 7.29 (d, J=2.0 Hz, 1H), 7.06 (dd, J=2.0 Hz, J=8.4 Hz, 1H), 5.67 (s, 2H), 4.97-4.92 (m, 1H), 3.76-3.73 (m, 2H), 3.56 (s, 3H), 3.44-3.41 (m, 4H), 3.38 (s, 3H), 3.36-3.34 (m, 3H), 3.31-3.29 (m, 4H), 3.32 (s, 3H), 3.00-2.94 (m, 1H), 2.85-2.74 (m, 4H), 2.60-2.57 (m, 4H), 2.27 (d, J=6.7 Hz, 2H), 2.13-2.08 (m, 1H), 1.83-1.79 (m, 2H), 1.22-1.11 (m, 4H).

¹³C NMR (101 MHz, CDCl3) δ 171.4, 169.1, 168.2, 167.5, 164.1, 164.0, 162.1, 156.3, 155.6, 155.0, 151.7, 147.9, 137.0, 134.4, 134.0, 131.5, 128.9, 126.2, 125.5, 123.1, 119.8, 118.0, 108.7, 105.0, 77.2, 64.4, 53.2 (2C), 50.5 (2C), 50.1, 47.6 (2C), 47.1 (2C), 46.5 (2C), 40.2, 33.9, 32.1, 30.8 (2C), 30.0, 28.0, 27.4, 22.2.

HPLC-MS t_{R}=1.708 min (98.7%), ESI+m/z 970.7936; 971.9340 [M+H]

### Development of Optimal Synthesis of Exemplary Compound

In the reaction above, a variety of conditions were assessed during step 1 (the reaction with methyl iodide). The results are given below.

Methyl iodide (3 equivalents) in acetonitrile at 60° C. yielded 80% conversion of starting material after five days.

Methyl iodide (6 equivalents) in acetonitrile at 60° C. yielded 80% conversion of starting material after eight days.

Methyl iodide (3 equivalents) in DMSO at 60° C. yielded 60% conversion when left overnight.

Methyl iodide (3 equivalents) in DMSO at 80° C. yielded some product when exposed to microwave heating for 15 minutes.

Methyl iodide (3 equivalents) in acetonitrile at 130° C. yielded 50% conversion of starting material when exposed to microwave heating for 15 minutes.

Methyl iodide (3 equivalents) in acetonitrile at 120° C. yielded 75% conversion of starting material when exposed to microwave heating for 2×25 minutes.

### Synthesis of Additional Exemplary Compounds and Intermediates

### General Procedure 1

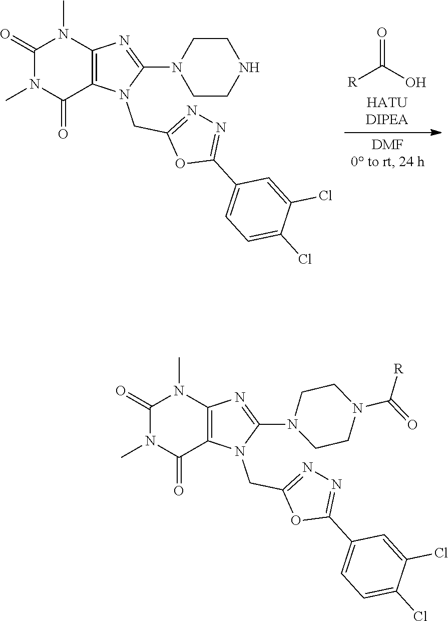

Under nitrogen, HATU was added to a solution of acid and DIPEA in DMF (0.010 M) at 0° C.

The mixture was stirred for 15 min at 0° C. The amine was then added portion wise. The mixture was stirred for 24 h at room temperature. After concentration under reduced pressure, the crude was purified by prep HPLC to afford the expected products.

### General Procedure 2

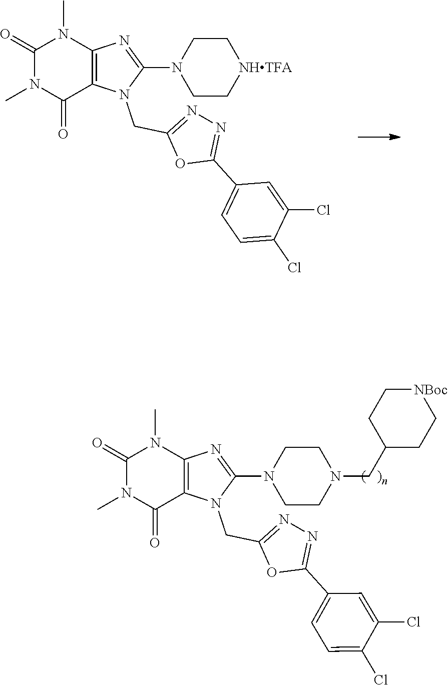

Under nitrogen, sodium triacetoxyborohydride was added to a solution of corresponding aldehyde and TH5427 trifluoroacetic salt in THF. The resulting mixture was stirred at room temperature overnight. The crude was purified by flash column chromatography (Si-35, DCM/MeOH, gradient from 100/0 to 95/5) to afford the expected product.

### General Procedure 3

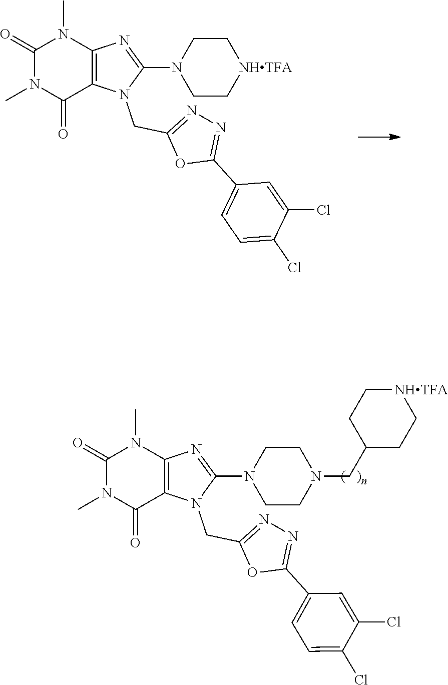

Under nitrogen, trifluoroacetic acid was added dropwise to a solution of N-Boc protected compound in DCM at 0° C. After completion, the crude was concentrated under reduced pressure and used in the next step without further purification.

### Synthesis of 7-((5-(3,4-dichlorophenyl)-1,3,4-oxadiazol-2-yl)methyl)-8-(4-(3-(2-(2-((2-(2,6-dioxopiperidin-3-yl)-1,3-dioxoisoindolin-4-yl)amino)ethoxy)ethoxy)propanoyl)piperazin-1-yl)-1,3-dimethyl-3,7-dihydro-1H-purine-2,6-dione

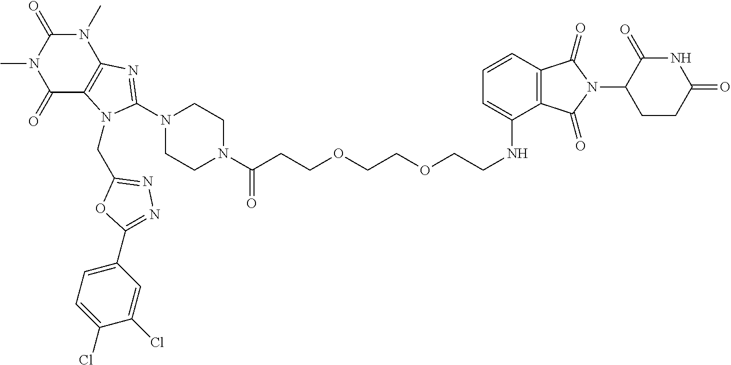 [SMILES: Cn1c(=O)c2c(nc(N3CCN(C(=O)CCOCCOCCNc4cccc5c4C(=O)N(C4CCC(=O)NC4=O)C5=O)CC3)n2Cc2nnc(-c3ccc(Cl)c(Cl)c3)o2)n(C)c1=O]

7-((5-(3,4-dichlorophenyl)-1,3,4-oxadiazol-2-yl)methyl)-8-(4-(3-(2-(2-((2-(2,6-dioxopiperidin-3-yl)-1,3-dioxoisoindolin-4-yl)amino)ethoxy)ethoxy)propanoyl)piperazin-1-yl)-1,3-dimethyl-3,7-dihydro-1H-purine-2,6-dione was prepared from TH5427 TFA salt (20 mg, 0.033 mmol, 1.1 eq), 3-(2-(2-((2-(2,6-dioxopiperidin-3-yl)-1,3-dioxoisoindolin-4-yl)amino)ethoxy)ethoxy) propanoic acid (13 mg, 0.030 mmol, 1 eq), DIPEA (21 μL, 0.12 mmol, 4 eq), HATU (23 mg, 0.060 mmol, 2 eq) and DMF (2.2 mL). Under nitrogen, HATU was added to a solution of acid and DIPEA in DMF at 0° C. The mixture was stirred for 15 min at 0° C. A solution of TH5427 TFA salt and DIPEA in DMF was then added dropwise. The mixture was stirred for 24 h at room temperature. After concentration under reduced pressure, the crude was purified by prep HPLC to afford the expected product as yellow solid (21.4 mg, 0.024 mmol, 79%).

¹H NMR (400 MHz, CDCl3) δ 8.88 (s, 1H), 8.07 (d, J=2.0 Hz, 1H), 7.83 (dd, J1=8.4 Hz, J2=2.0 Hz, 1H), 7.57 (d, J=8.4 Hz, 1H), 7.45 (dd, J1=7.0 Hz, J2=8.4 Hz, 1H), 7.06 (d, J=7.0 Hz, 1H), 6.88 (d, J=8.4 Hz, 1H), 6.45 (bs, 1H), 5.68 (s, 2H), 4.93-4.88 (m, 1H), 3.79 (t, J=6.5 Hz, 2H), 3.71 (bt, J=5.3 Hz, 4H), 3.65-3.63 (m, 4H), 3.61-3.59 (m, 2H), 3.53 (s, 3H), 3.46-3.42 (m, 2H), 3.35 (s, 3H), 3.29-3.23 (m, 4H), 2.88-2.81 (m, 1H), 2.77-2.72 (m, 2H), 2.64-2.60 (m, 2H), 2.17-2.10 (m, 1H).

¹³C NMR (101 MHz, CDCl₃) δ 171.5, 170.1, 169.4, 168.8, 167.6, 164.1, 162.1, 155.9, 155.0, 151.6, 147.7, 146.88, 136.9, 136.1, 133.9, 132.5, 131.5, 128.8, 126.1, 123.0, 116.9, 111.7, 110.3, 104.9, 70.6, 70.5, 69.5, 67.7, 50.8, 50.3, 49.00, 45.2, 42.5, 41.0, 40.1, 33.7, 31.5, 30.0, 28.0, 22.9.

HPLC-MS t_{R}=1.542 min (100%), ESI+m/z 907.379 [M+H]⁺

HRMS was calculated for C₄₀H₄₂Cl₂N₁₁O₁₀: 906.2493, found: 906.2487.

### Synthesis of 7-((5-(3,4-dichlorophenyl)-1,3,4-oxadiazol-2-yl)methyl)-8-(4-(3-(2-(2-(2-((2-(2,6-dioxopiperidin-3-yl)-1,3-dioxoisoindolin-4-yl)amino)ethoxy)ethoxy)ethoxy)propanoyl)piperazin-1-yl)-1,3-dimethyl-3,7-dihydro-1H-purine-2,6-dione

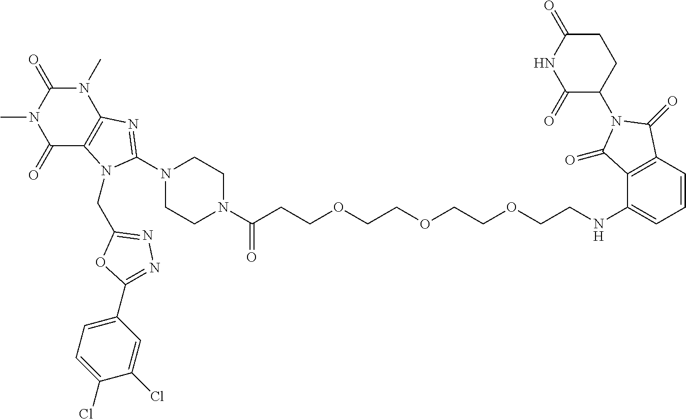 [SMILES: Cn1c(=O)c2c(nc(N3CCN(C(=O)CCOCCOCCOCCNc4cccc5c4C(=O)N(C4CCC(=O)NC4=O)C5=O)CC3)n2Cc2nnc(-c3ccc(Cl)c(Cl)c3)o2)n(C)c1=O]

7-((5-(3,4-dichlorophenyl)-1,3,4-oxadiazol-2-yl)methyl)-8-(4-(3-(2-(2-(2-((2-(2,6-dioxopiperidin-3-yl)-1,3-dioxoisoindolin-4-yl)amino)ethoxy)ethoxy)ethoxy)propanoyl)piperazin-1-yl)-1,3-dimethyl-3,7-dihydro-1H-purine-2,6-dione was prepared according to the general procedure 1 using TH5427 (26.9 mg, 0.055 mmol, 1.1 eq), 3-(2-(2-(2-((2-(2,6-dioxopiperidin-3-yl)-1,3-dioxoisoindolin-4-yl)amino)ethoxy)ethoxy)ethoxy)propanoic acid (23.8 mg, 0.050 mmol, 1 eq), DIPEA (17 μL, 0.1 mmol, 2 eq) and HATU (37.9 mg, 0.1 mmol, 2 eq). After purification, a yellow solid was obtained (14.9 mg, 0.016 mmol, 31%).

¹H NMR (400 MHz, CDCl3) δ 8.86 (s, 1H), 8.10 (d, J=2 Hz, 1H), 7.86 (dd, J1=2.0 Hz, J2=8.5 Hz, 1H), 7.59 (d, J=8.5 Hz, 1H) 7.48 (dd, J1=7.0 Hz, J2=8.5 Hz, 1H) 7.09 (d, J=7.0 Hz, 1H) 6.90 (d, J=8.5 Hz, 1H), 5.68 (s, 2H), 4.94-4.89 (m, 1H), 3.80 (t, J=6.5, 2H), 3.75-3.70 (m, 4H), 3.68-3.60 (m, 10H), 3.54 (s, 3H), 3.47-3.44 (m, 2H), 3.37 (s, 3H), 3.31-3.24 (m, 4H), 2.89-2.82 (m, 1H), 2.79-2.72 (m, 2H), 2.64 (t, J=6.5, 2H), 2.17-2.10 (m, 1H). NH—Ar not observed

¹³C NMR (101 MHz, CDCl₃) δ 171.5, 170.2, 169.4, 168.7, 167.7, 164.1, 162.1, 155.9, 155.0, 151.7, 147.8, 146.9, 136.9, 136.1, 134.0, 132.7, 131.5, 128.9, 126.2, 123.1, 116.8, 111.8, 110.5, 104.9, 71.0, 70.7, 70.6, 70.5, 69.5, 67.5, 50.6, 50.5, 49.0, 45.2, 42.5, 41.0, 40.1, 33.7, 31.6, 30.0, 28.0, 23.0.

HPLC-MS t_{R}=1.558 min (96%), ESI+m/z 951.535 [M+H]⁺

HRMS was calculated for C₄₂H₄₆Cl₂N₁₁O₁₁: 950.2755, found: 950.2727.

### Synthesis of 7-((5-(3,4-dichlorophenyl)-1,3,4-oxadiazol-2-yl)methyl)-8-(4-(1-((2-(2,6-dioxopiperidin-3-yl)-1,3-dioxoisoindolin-4-yl)amino)-3,6,9,12-tetraoxapentadecan-15-oyl)piperazin-1-yl)-1,3-dimethyl-3,7-dihydro-1H-purine-2,6-dione

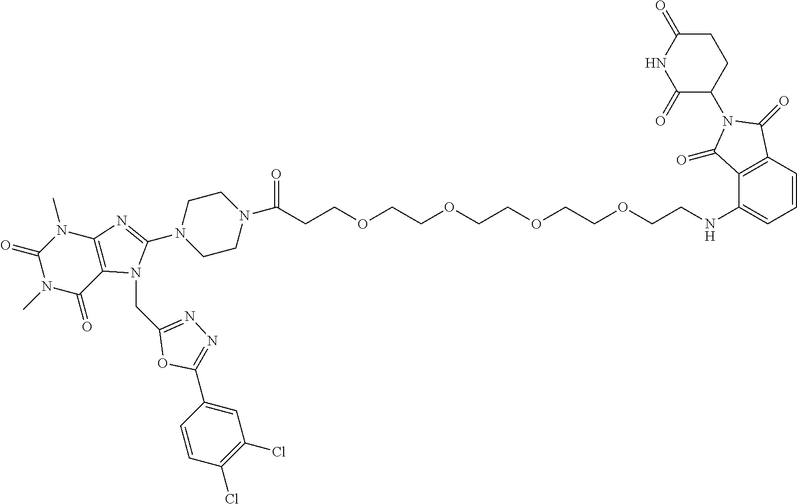 [SMILES: Cn1c(=O)c2c(nc(N3CCN(C(=O)CCOCCOCCOCCOCCNc4cccc5c4C(=O)N(C4CCC(=O)NC4=O)C5=O)CC3)n2Cc2nnc(-c3ccc(Cl)c(Cl)c3)o2)n(C)c1=O]

7-((5-(3,4-dichlorophenyl)-1,3,4-oxadiazol-2-yl)methyl)-8-(4-(1-((2-(2,6-dioxopiperidin-3-yl)-1,3-dioxoisoindolin-4-yl)amino)-3,6,9,12-tetraoxapentadecan-15-oyl)piperazin-1-yl)-1,3-dimethyl-3,7-dihydro-1H-purine-2,6-dione was prepared according to the general procedure 1 using TH5427 (10.3 mg, 0.021 mmol, 1.1 eq), 1-((2-(2,6-dioxopiperidin-3-yl)-1,3-dioxoisoindolin-4-yl)amino)-3,6,9,12-tetraoxa pentadecan-15-oic acid (9.9 mg, 0.019 mmol, 1 eq), DIPEA (10 μL, 0.057 mmol, 3 eq) and HATU (14.4 mg, 0.038 mmol, 2 eq). After purification, a yellow solid was obtained (4.9 mg, 0.005 mmol, 26%).

¹H NMR (400 MHz, CDCl₃) δ 8.57 (s, 1H), 8.11 (d, J=2 Hz, 1H), 7.86 (dd, J1=2.0 Hz, J2=8.5 Hz, 1H), 7.60 (d, J=8.5 Hz, 1H) 7.49 (dd, J1=7.0 Hz, J2=8.5 Hz, 1H) 7.10 (d, J=7.0 Hz, 1H) 6.92 (d, J=8.5 Hz, 1H), 5.69 (s, 2H), 4.93-4.89 (m, 1H), 3.81 (t, J=6.5, 2H), 3.74-3.71 (m, 4H), 3.68-3.64 (m, 8H), 3.64-3.61 (m, 6H), 3.56 (s, 3H), 3.47 (t, J=5.5 Hz, 2H), 3.38 (s, 3H), 3.30-3.27 (m, 4H), 2.90-2.84 (m, 1H), 2.81-2.72 (m, 2H), 2.65 (t, J=6.5, 2H), 2.16-2.10 (m, 1H). NH—Ar not observed

¹³C NMR (101 MHz, CDCl₃) δ 171.3, 170.0, 169.4, 168.6, 167.7, 164.1, 162.1, 155.9, 155.0, 151.7, 147.8, 146.9, 136.9, 136.2, 134.0, 132.7, 131.5, 128.9, 126.2, 123.1, 116.9, 111.8, 110.5, 105.0, 70.9, 70.8, 70.7, 70.6, 70.6, 70.5, 69.6, 67.5, 50.7, 50.5, 49.0, 45.2, 42.5, 41.0, 40.1, 33.7, 31.6, 30.0, 28.0, 23.0.

HPLC-MS t_{R}=1.550 min (97.8%), ESI+m/z 995.315; 996.395 [M+H]+

HRMS was calculated for C₄₄H₅₀Cl₂N₁₁O₁₂: 994.3018, found: 994.3007.

### Synthesis of tert-butyl 4-(2-(4-(7-((5-(3,4-dichlorophenyl)-1,3,4-oxadiazol-2-yl)methyl)-1,3-dimethyl-2,6-dioxo-2,3,6,7-tetrahydro-1H-purin-8-yl)piperazin-1-yl)ethyl)piperidine-1-carboxylate

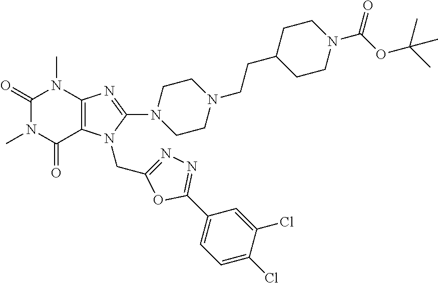 [SMILES: Cn1c(=O)c2c(nc(N3CCN(CCC4CCN(C(=O)OC(C)(C)C)CC4)CC3)n2Cc2nnc(-c3ccc(Cl)c(Cl)c3)o2)n(C)c1=O]

Tert-butyl 4-(2-(4-(7-((5-(3,4-dichlorophenyl)-1,3,4-oxadiazol-2-yl)methyl)-1,3-dimethyl-2,6-dioxo-2,3,6,7-tetrahydro-1H-purin-8-yl)piperazin-1-yl)ethyl)piperidine-1-carboxylate was prepared according to the general procedure 2 using TH5427.TFA (33 mg, 0.055 mmol, 1 eq), N-Boc-4-piperidineacetaldehyde (14.9 mg, 0.065 mmol, 1.2 eq), sodium triacetoxyborohydride (17.3 mg, 0.082 mmol, 1.5 eq) and THF (1 mL). After purification, a light brown solid was obtained (33.1 mg, 0.047 mmol, 86%).

¹H NMR (400 MHz, CDCl₃) δ 8.08 (d, J=2.0 Hz, 1H), 7.83 (dd, J=2.0 Hz, J=8.4 Hz, 1H), 7.57 (d, J=8.4 Hz, 1H), 5.62 (s, 2H), 4.05 (bs, 3H), 3.53 (s, 3H), 3.34 (s, 3H), 3.33-3.32 (m, 4H), 2.68-2.61 (m, 3H) 2.58-2.55 (bs, 4H), 2.44-2.41 (m, 2H), 1.66-1.59 (m, 3H), 1.43 (s, 9H), 1.15-1.04 (m, 3H).

¹³C NMR (101 MHz, CDCl₃) δ 164.0, 162.2, 156.5, 155.0, 154.9, 151.7, 148.0, 136.8, 133.9, 131.5, 128.9, 126.1, 123.2, 104.8, 79.4, 60.3, 55.9, 52.3 (2C), 50.1 (2C), 40.3 (2C), 39.4, 34.4, 32.6, 32.3 (2C), 30.0, 28.6 (3C), 27.9.

HPLC-MS t_{R}=1.858 min (94.5%), ESI+m/z 702.824; 705.992 [M+H]⁺

### Synthesis of 4-(2-(4-(7-((5-(3,4-dichlorophenyl)-1,3,4-oxadiazol-2-yl)methyl)-1,3-dimethyl-2,6-dioxo-2,3,6,7-tetrahydro-1H-purin-8-yl)piperazin-1-yl)ethyl)piperidin-1-ium trifluoroacetate

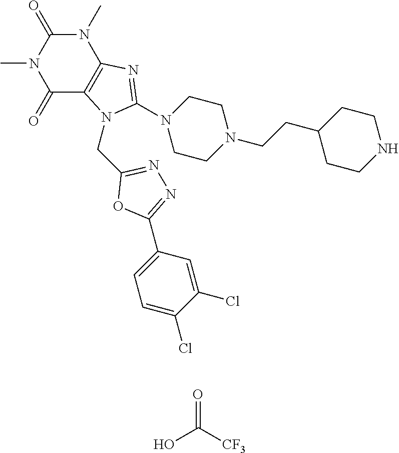 [SMILES: Cn1c(=O)c2c(nc(N3CCN(CCC4CCNCC4)CC3)n2Cc2nnc(-c3ccc(Cl)c(Cl)c3)o2)n(C)c1=O ; O=C(O)C(F)(F)F]

4-(2-(4-(7-((5-(3,4-dichlorophenyl)-1,3,4-oxadiazol-2-yl)methyl)-1,3-dimethyl-2,6-dioxo-2,3,6,7-tetrahydro-1H-purin-8-yl)piperazin-1-yl)ethyl)piperidin-1-ium trifluoroacetate was prepared from tert-butyl 4-((4-(7-((5-(3,4-dichlorophenyl)-1,3,4-oxadiazol-2-yl)methyl)-1,3-dimethyl-2,6-dioxo-2,3,6,7-tetrahydro-1H-purin-8-yl)piperazin-1-yl)ethyl)piperidine-1-carboxylate (33 mg, 0.047 mmol, 1 eq), TFA (109 μL, 1.410 mmol, 30 eq) and DCM (2.8 mL) using the general procedure 3. The crude was used directly in the next step without further purification.

¹H NMR (400 MHz, MeOD) δ 8.15-8.14 (d, J=2.0 Hz, 1H), 7.92 (dd, J=2.0 Hz, J=8.4 Hz, 1H), 7.73 (d, J=8.4 Hz, 1H), 5.78 (s, 2H), 3.79-3.77 (m, 3H), 3.51 (s, 3H), 3.43-3.36 (m, 8H), 3.30 (s, 3H), 3.03-2.98 (m, 4H), 2.02-2.01 (m, 2H), 1.85-1.80 (m, 2H), 1.55-1.51 (m, 2H) (NH. TFA not observed).

### Synthesis of 7-((5-(3,4-dichlorophenyl)-1,3,4-oxadiazol-2-yl)methyl)-8-(4-(2-(1-(2-(2,6-dioxopiperidin-3-yl)-1,3-dioxoisoindolin-5-yl)piperidin-4-yl)ethyl)piperazin-1-yl)-1,3-dimethyl-3,7-dihydro-1H-purine-2,6-dione

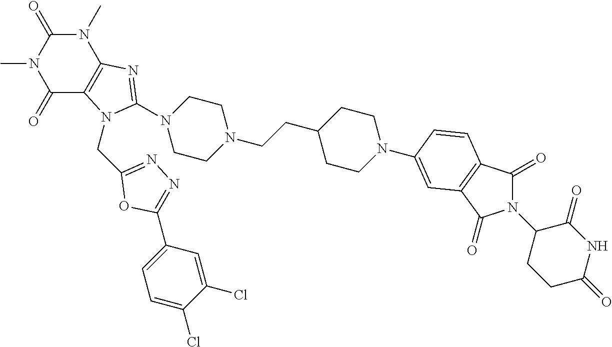 [SMILES: Cn1c(=O)c2c(nc(N3CCN(CCC4CCN(c5ccc6c(c5)C(=O)N(C5CCC(=O)NC5=O)C6=O)CC4)CC3)n2Cc2nnc(-c3ccc(Cl)c(Cl)c3)o2)n(C)c1=O]

7-((5-(3,4-dichlorophenyl)-1,3,4-oxadiazol-2-yl)methyl)-8-(4-(2-(1-(2-(2,6-dioxopiperidin-3-yl)-1,3-dioxoisoindolin-5-yl)piperidin-4-yl)ethyl)piperazin-1-yl)-1,3-dimethyl-3,7-dihydro-1H-purine-2,6-dione was prepared from 7-((5-(3,4-dichlorophenyl)-1,3,4-oxadiazol-2-yl)methyl)-1,3-dimethyl-8-(4-(2-(piperidin-4-yl)ethyl)piperazin-1-yl)-3,7-dihydro-1H-purine-2,6-dione trifluoroacetic salt (33 mg, 0.046 mmol, 1 eq), 5-fluoro thalidomide (12.7 mg, 0.046 mmol, 1 eq), TEA (19 μL, 0.138 mmol, 3 eq) and DMSO (1.2 mL). Under nitrogen, TEA was added to a solution of the amine (trifluoroacetic salt) and fluoro thalidomide in DMSO. The mixture was heated under microwave at 200° C. for 20 min. The crude was purified by prep HPLC to afford the expected product as yellow solid (1.7 mg, 0.002 mmol, 4%).

¹H NMR (400 MHz, CDCl₃) δ 8.12 (d, J=2.0 Hz, 1H), 8.00 (bs, 1H), 7.86 (dd, J=2.0 Hz, J=8.5 Hz, 1H), 7.67 (d, J=8.4 Hz, 1H) 7.61 (d, J=8.4 Hz), 7.05-7.02 (m, 1H), 5.65 (s, 2H), 4.96-4.91 (m, 1H), 3.96-3.93 (m, 2H), 3.55 (s, 3H), 3.37 (s, 3H), 3.00-2.92 (m, 4H), 2.88-2.70 (m, 5H), 2.16-2.12 (m, 2H), 1.85-1.82 (m, 3H), 1.62-1.58 (m, 9H).

HRMS was calculated for C₄₀H₄₂Cl₂N₁₁O₇: 858.2646, found: 858.2644.

### Synthesis of tert-butyl 4-((4-(7-((5-(3,4-dichlorophenyl)-1,3,4-oxadiazol-2-yl)methyl)-1,3-dimethyl-2,6-dioxo-2,3,6,7-tetrahydro-1H-purin-8-yl)piperazin-1-yl)methyl)piperidine-1-carboxylate

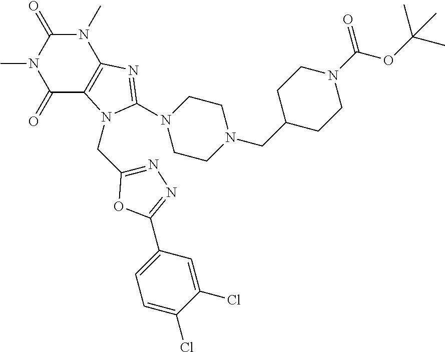 [SMILES: Cn1c(=O)c2c(nc(N3CCN(CC4CCN(C(=O)OC(C)(C)C)CC4)CC3)n2Cc2nnc(-c3ccc(Cl)c(Cl)c3)o2)n(C)c1=O]

Tert-butyl 4-((4-(7-((5-(3,4-dichlorophenyl)-1,3,4-oxadiazol-2-yl)methyl)-1,3-dimethyl-2,6-dioxo-2,3,6,7-tetrahydro-1H-purin-8-yl)piperazin-1-yl)methyl)piperidine-1-carboxylate was prepared according to the general procedure 2 using TH5427.TFA (50 mg, 0.083 mmol, 1 eq), 1-Boc-piperidine-4-carboxaldehyde (21.1 mg, 0.099 mmol, 1.2 eq), sodium triacetoxyborohydride (26.3 mg, 0.124 mmol, 1.5 eq) and THF (1.5 mL). After purification, an off-white solid was obtained (40 mg, 0.058 mmol, 70%).

¹H NMR (400 MHz, CDCl₃) δ 8.08 (d, J=2.0 Hz, 1H), 7.83 (dd, J=2.0 Hz, J=8.4 Hz, 1H), 7.57 (d, J=8.4 Hz, 1H), 5.63 (s, 2H), 4.06 (bs, 2H), 3.53 (s, 3H), 3.34 (s, 3H), 3.30-3.27 (m, 4H), 2.66 (t, J=12.7, 2H) 2.49 (bs, 4H), 2.19 (d, J=6.7 Hz, 2H), 1.70 (bd, J=13.2 Hz, 2H), 1.61-1.59 (m, 1H), 1.43 (s, 9H), 1.10-1.00 (m, 2H).

¹³C NMR (101 MHz, CDCl₃) δ 163.9, 162.2, 156.7, 155.0, 154.9, 151.7, 148.0, 136.8, 133.9, 131.4, 128.8, 126.1, 123.2, 104.8, 79.4, 64.4, 52.9 (2C), 50.5 (2C), 40.3 (2C), 33.6, 30.7 (2C), 29.9, 28.6 (3C), 27.9.

### Synthesis of 4-((4-(7-((5-(3,4-dichlorophenyl)-1,3,4-oxadiazol-2-yl)methyl)-1,3-dimethyl-2,6-dioxo-2,3,6,7-tetrahydro-1H-purin-8-yl)piperazin-1-yl)methyl)piperidin-1-ium trifluoroacetate

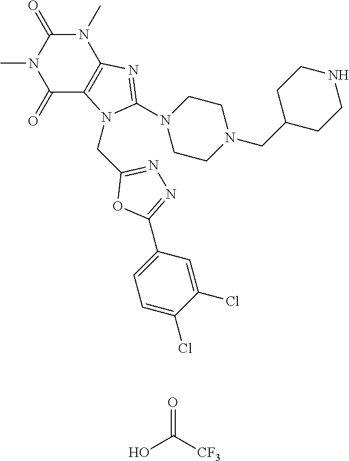 [SMILES: Cn1c(=O)c2c(nc(N3CCN(CC4CCNCC4)CC3)n2Cc2nnc(-c3ccc(Cl)c(Cl)c3)o2)n(C)c1=O ; O=C(O)C(F)(F)F]

4-((4-(7-((5-(3,4-dichlorophenyl)-1,3,4-oxadiazol-2-yl)methyl)-1,3-dimethyl-2,6-dioxo-2,3,6,7-tetrahydro-1H-purin-8-yl)piperazin-1-yl)methyl)piperidin-1-ium trifluoroacetate was prepared from tert-butyl 4-((4-(7-((5-(3,4-dichlorophenyl)-1,3,4-oxadiazol-2-yl)methyl)-1,3-dimethyl-2,6-dioxo-2,3,6,7-tetrahydro-1H-purin-8-yl)piperazin-1-yl)methyl)piperidine-1-carboxylate (40 mg, 0.058 mmol, 1 eq), TFA (134 μL, 1.743 mmol, 30 eq) and DCM (2.5 mL) using the general procedure 3. The crude was used directly in the next step without further purification.

¹H NMR (400 MHz, MeOD) δ 8.12-8.11 (d, J=2.0 Hz, 1H), 7.90 (dd, J=2.0 Hz, J=8.4 Hz, 1H), 7.71 (d, J=8.4 Hz, 1H), 5.77 (s, 2H), 3.71-3.59 (m, 7H), 3.49 (s, 3H), 3.48-3.42 (m, 3H), 3.24 (s, 3H), 3.23-3.21 (m, 2H), 3.10-3.01 (m, 2H), 2.35-2.27 (m, 1H), 2.11-2.07 (m, 2H), 1.63-1.50 (m, 2H) (NH.TFA not observed).

HPLC-MS t_{R}=1.325 min (90.9%), ESI+m/z 588.714; 590.634 [M+H]⁺ (free NH)

### Synthesis of 7-((5-(3,4-dichlorophenyl)-1,3,4-oxadiazol-2-yl)methyl)-8-(4-((1-(2-(2,6-dioxopiperidin-3-yl)-1,3-dioxoisoindolin-5-yl)piperidin-4-yl)methyl)piperazin-1-yl)-1,3-dimethyl-3,7-dihydro-1H-purine-2,6-dione

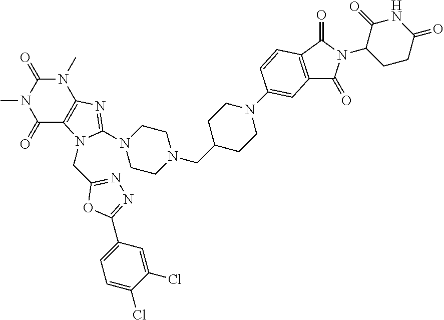 [SMILES: Cn1c(=O)c2c(nc(N3CCN(CC4CCN(c5ccc6c(c5)C(=O)N(C5CCC(=O)NC5=O)C6=O)CC4)CC3)n2Cc2nnc(-c3ccc(Cl)c(Cl)c3)o2)n(C)c1=O]

7-((5-(3,4-dichlorophenyl)-1,3,4-oxadiazol-2-yl)methyl)-8-(4-((1-(2-(2,6-dioxopiperidin-3-yl)-1,3-dioxoisoindolin-5-yl)piperidin-4-yl)methyl)piperazin-1-yl)-1,3-dimethyl-3,7-dihydro-1H-purine-2,6-dione was prepared from 7-((5-(3,4-dichlorophenyl)-1,3,4-oxadiazol-2-yl)methyl)-1,3-dimethyl-8-(4-(piperidin-4-ylmethyl)piperazin-1-yl)-3,7-dihydro-1H-purine-2,6-dione trifluoroacetic salt (27 mg, 0.038 mmol, 1 eq), 5-fluoro thalidomide (10.6 mg, 0.038 mmol, 1 eq), DIPEA (14 μL, 0.098 mmol, 3 eq) and DMSO (1.1 mL). Under nitrogen, DIPEA was added to a solution of the amine (trifluoroacetic salt) and fluoro thalidomide in DMSO. The mixture was heated at 100° C. overnight. The crude was purified by prep HPLC to afford the expected product as yellow solid (7.4 mg, 0.009 mmol, 23%).

¹H NMR (400 MHz, CDCl₃) δ 8.18 (s, 1H), 8.10 (d, J=2.0 Hz, 1H), 7.85 (dd, J=2.0 Hz, J=8.5 Hz, 1H), 7.66 (d, J=8.5 Hz, 1H) 7.59 (d, J=8.5 Hz, 1H), 7.27 (d, J=2.3 Hz, 1H), 7.03 (dd, J=2.3 Hz, J=8.5 Hz, 1H), 5.65 (s, 2H), 4.95-4.91 (m, 1H), 3.96-3.93 (m, 2H), 3.56 (s, 3H), 3.37 (s, 3H), 3.32 (bs, 3H), 2.98-2.90 (m, 2H), 2.86-2.68 (m, 4H), 2.53 (bs, 4H), 2.25 (bs, 2H), 2.14-2.09 (m, 1H), 1.90-1.87 (m, 3H), 1.74, 1.32-1.26 (m, 2H).

¹³C NMR (101 MHz, CDCl₃) δ 171.2, 168.5, 168.2, 167.4, 164.0, 162.2, 156.7, 155.5, 155.0, 151.8, 148.1, 136.9, 134.5, 134.0, 131.5, 128.9, 126.2, 125.6, 123.2, 118.7, 118.0, 108.8, 104.9, 64.2, 53.0 (2C), 50.5 (2C), 49.3, 48.1 (2C), 40.3, 33.4, 31.6, 30.1 (2C), 30.0, 28.0, 22.9.

HPLC-MS t_{R}=1.683 min (96.6%), ESI+m/z 844.795; 845.995 [M+H]

HRMS was calculated for C₃₉H₄₀Cl₂N₁₁O₇: 844.2489, found: 844.2483.

### Identification of Alternative X Groups

To identify alternative X groups, a screening exercise was performed. A selection of BTK inhibitors was tested alongside TH5427, a known selective NUDT5 inhibitor in a catalytic assay. Inhibition activities of the compounds were determined using AMP-Glo system (Promega). The compounds were diluted from 50 μM to 0 μM in a final reaction containing 20 mM Hepes, 100 mM NaCl, 0.5 mM TCEP, 1 mM MgCl2 and 0.1% BSA, pH 7.4. The reactions were performed in 1536 well plate in 2 pl reaction volume with 2 nM of NUDT5 and NUDT14, and 10 M of ADPr as the substrate. The final DMSO concentration was 0.5% for all reactions. NUDT5 reactions were incubated for 20 minutes while NUDT14 reactions were carried out for 1 hour at room temperature. The reactions were stopped using 2 l of AMP-Glo I. The stop solution is supplemented with 25 M of a compound, PubChem CID 16339098, in order to stop the activity of the proteins completely. The reactions were further incubated with the 4 μl of Detection solution for 1 hour at room temperature. Luminescence signals were then measured in PHERAstar FSX plate reader. Experiments were done in triplicate sets and data were analysed by GraphPad Prism 9. Data are represented as the mean±SD of three biological experiments:

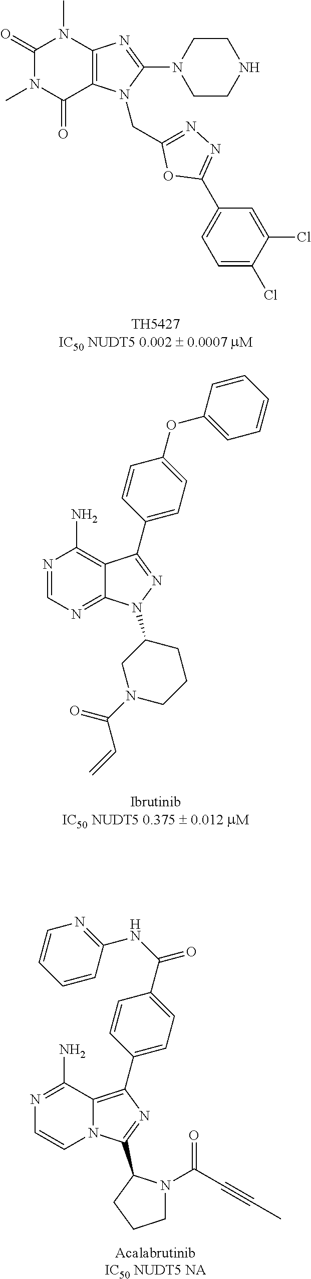 [SMILES: Cn1c(=O)c2c(nc(N3CCNCC3)n2Cc2nnc(-c3ccc(Cl)c(Cl)c3)o2)n(C)c1=O ; C=CC(=O)N1CCC[C@@H](n2nc(-c3ccc(Oc4ccccc4)cc3)c3c(N)ncnc32)C1 ; CC#CC(=O)N1CCC[C@H]1c1nc(-c2ccc(C(=O)Nc3ccccn3)cc2)c2c(N)nccn12]

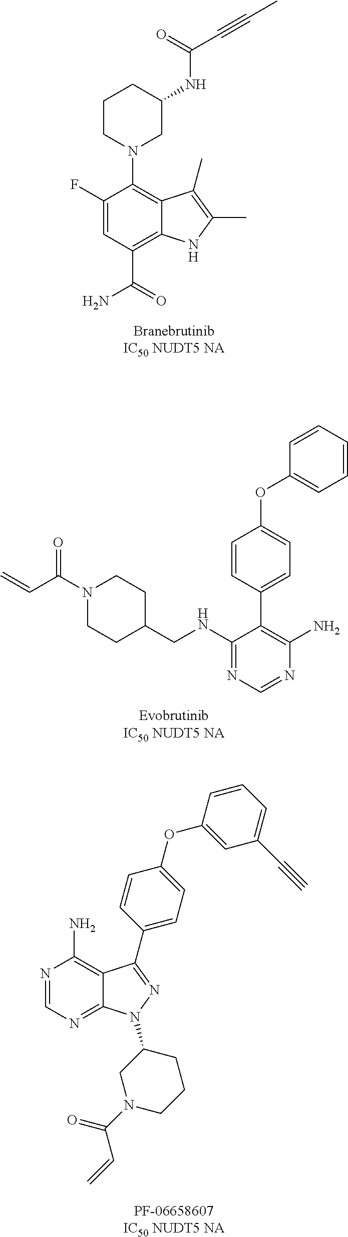 [SMILES: CC#CC(=O)N[C@H]1CCCN(c2c(F)cc(C(N)=O)c3[nH]c(C)c(C)c23)C1 ; C=CC(=O)N1CCC(CNc2ncnc(N)c2-c2ccc(Oc3ccccc3)cc2)CC1 ; C#Cc1cccc(Oc2ccc(-c3nn([C@@H]4CCCN(C(=O)C=C)C4)c4ncnc(N)c34)cc2)c1]

The catalytic assay results showed that ibrutinib was the only active compound among the BTK inhibitors tested with a suitable ability to bind NUDT5. Accordingly, Ibrutinib is suggested as a suitable X group.

Further, an additional assay was performed to identify analogues of Ibrutinib which may be suitable X groups. The assay tested the ability of the analogues to bind NUDT14 as well as NUDT5. Within the NUDIX family, NUDT14 is closely related to NUDT5 as both have been shown to hydrolyse ADP-glucose and ADP-ribose. Although they hydrolyse similar substrates, they share low sequence similarity and possess structural differences. In order to assess these differences, we verified whether ibrutinib was able to exhibit any activity against this family member. Interestingly, ibrutinib inhibited NUDT14 catalytic activity with an IC₅₀ value of around 1 M.

The synthetic route of compounds is described in Scheme 1. Briefly, synthesis started with commercially available compound 9 which was further functionalized at N1- or C3-position. A methylpiperidin-4-yl moiety and a methyl group were added at N1-position via Mitsunobu or SN2 reaction followed by a Suzuki Miyaura coupling to obtain 1 and 2.

Compound 3 was prepared in one step from 9 using the same Suzuki Miyaura coupling conditions. Compound 10a was also engaged in the same transformation with different boronic acids or esters to afford compounds 11a-d. A subsequent deprotection of the Boc group followed by an amide coupling afforded the acrylamide-containing products 4, 5 and 6. For compound 7, a saturated analogue of 4, a hydrogenation step was performed before the deprotection step. Finally, compound 3 was obtained after reduction of the nitro group in 1Id followed by amide coupling with chloroacetyl chloride.

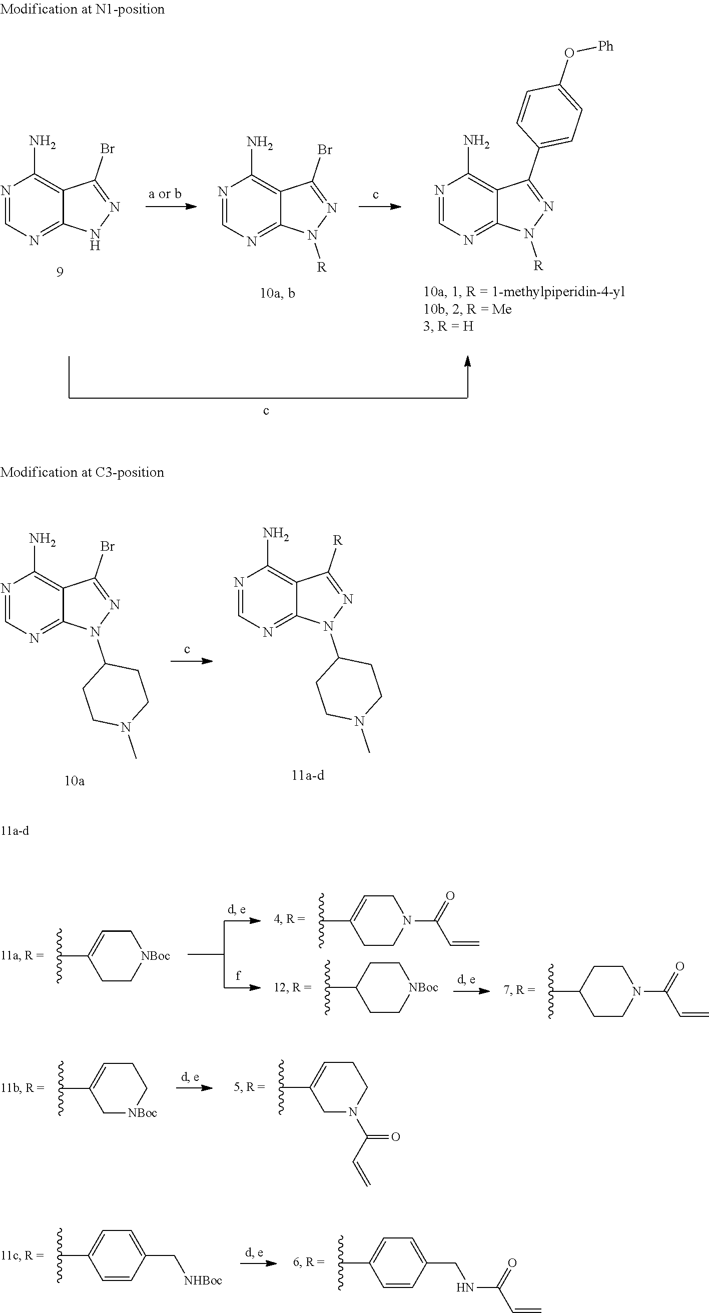

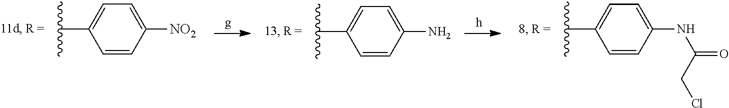 [SMILES: *c1ccc([N+](=O)[O-])cc1 ; *c1ccc(N)cc1 ; *c1ccc(NC(=O)CCl)cc1]

The reagents and conditions for each step were:
(a) 4-Hydroxy-1-methylpiperidine, PS-PPh₃, DIAD, THF, rt, 12 h;
(b) MeI, Cs₂CO₃, DMF, rt, 12 h;
(c) R—B(OH)₂ or R-BPin, Pd(dppf)Cl₂·CH₂Cl₂, Na₂CO₃, 1,4-dioxane/H₂O, 80° C., 12 h;
(d) 4 N HCl in 1,4-dioxane/CH₂Cl₂, rt, 2 h;
(e) acryloyl chloride, NEt₃, CH₂Cl₂, rt, 30 min;
(f) Pd/C, H₂, MeOH, 40° C., 12 h;
(g) Pd/C, H₂, MeOH, r.t., 6 h;
(h) chloroacetyl chloride, NEt₃, CH₂Cl₂, rt

The resulting compounds have general formula:

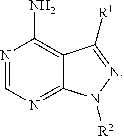

All compounds were tested in the above-described catalytic assay for their ability to bind both proteins. The results are shown in table 1, below.

TABLE 1

| | | | NUDT5 | NUDT14 |
|---|---|---|---|---|
| Compound | | | IC₅₀ | IC₅₀ |
| number | R¹ | R² | (μM) | (μM) |
| Ibrutinib | 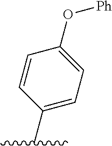 [SMILES: *c1ccc(Oc2ccccc2)cc1] | 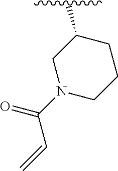 [SMILES: *[C@@H]1CCCN(C(=O)C=C)C1] | 0.375 ± 0.012 | 0.974 ± 0.026 |
| 1 | 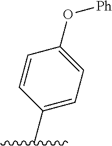 [SMILES: *c1ccc(Oc2ccccc2)cc1] | 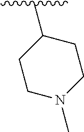 [SMILES: *C1CCN(C)CC1] | 0.259 ± 0.008 | 0.131 ± 0.002 |
| 2 | 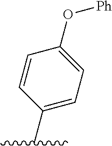 [SMILES: *c1ccc(Oc2ccccc2)cc1] | Me | 0.468 ± 0.007 | 0.604 ± 0.116 |
| 3 | 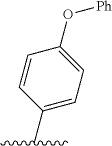 [SMILES: *c1ccc(Oc2ccccc2)cc1] | H | 0.480 ± 0.035 | 0.548 ± 0.037 |
| 4 | 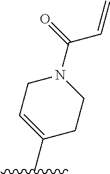 [SMILES: *C1=CCN(C(=O)C=C)CC1] | 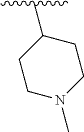 [SMILES: *C1CCN(C)CC1] | NA | NA |
| 5 | 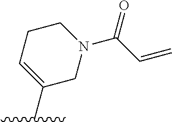 [SMILES: *C1=CCCN(C(=O)C=C)C1] | 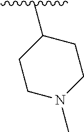 [SMILES: *C1CCN(C)CC1] | NA | 4.265 ± 0.133 |
| 6 | 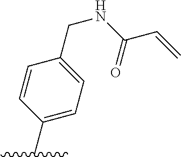 [SMILES: *c1ccc(CNC(=O)C=C)cc1] | 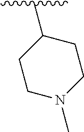 [SMILES: *C1CCN(C)CC1] | 14.524 ± 0.359 | 1.653 ± 0.095 |
| 7 | 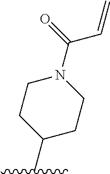 [SMILES: *C1CCN(C(=O)C=C)CC1] | 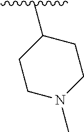 [SMILES: *C1CCN(C)CC1] | NA | NA |
| 8 | 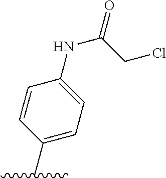 [SMILES: *c1ccc(NC(=O)CCl)cc1] | 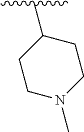 [SMILES: *C1CCN(C)CC1] | NA | 0.377 ± 0.035 |

### Ability of Compounds to Degrade NUDT5

A series of compounds were tested for their ability to degrade NUDT5. The compounds were of the following structures:

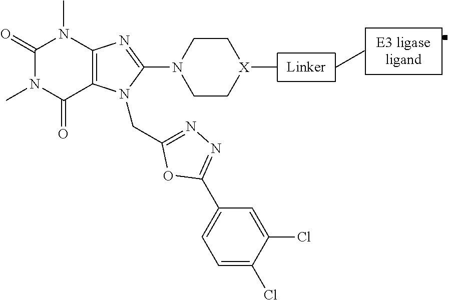

| | | | | NUDT5 |
|---|---|---|---|---|
| | | | | degra- |
| | | | | dation |
| | X as | | | at 100 |
| | shown | | | nM, |
| Cpd ID | above | Linker | E3 ligase ligand | 20 h (%) |
| 076 | N | 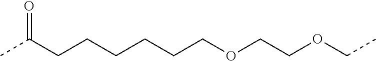 | 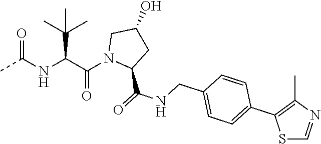 | NT |
| 131 | N | 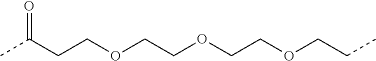 | 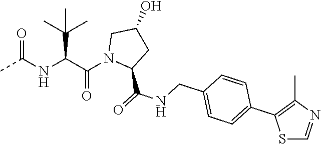 | NT |
| 063 | CH | 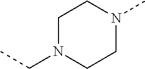 | 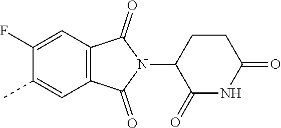 | NT |
| 056 | CH | 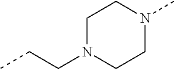 | 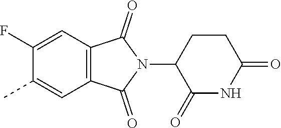 | NT |
| 084 | N | 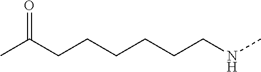 | 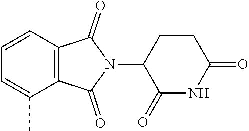 | 20 |
| 102 | N | 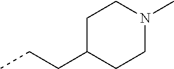 | 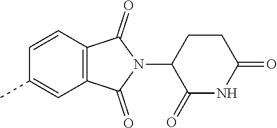 | 46 |
| 105 | N | 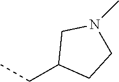 | 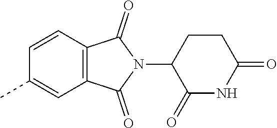 | NT |
| 110 | N | 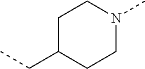 | 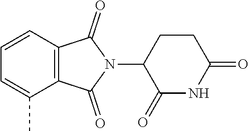 | NT |
| 111 | N | 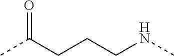 | 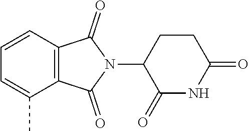 | 0 |
| 057 | N | 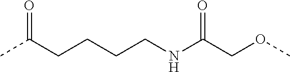 | 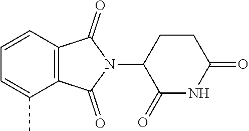 | NT |
| 068 | N | 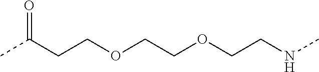 | 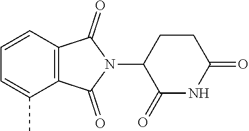 | 52 |
| 070 | N | 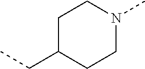 | 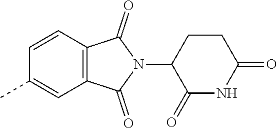 | 39 |
| 071 | N | 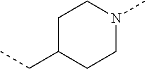 | 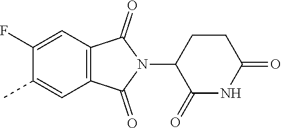 | NT |
| 122 | N | 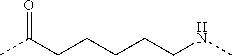 | 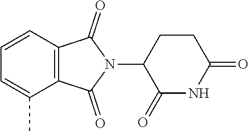 | 15 |
| 124 | N | 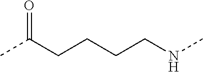 | 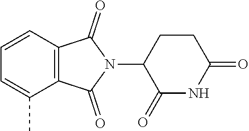 | 6 |
| 127 | N | 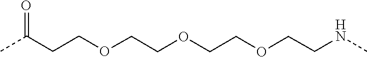 | 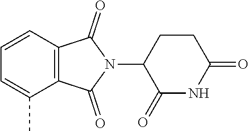 | 76 |
| 130 | N | 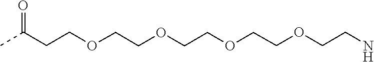 | 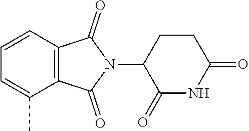 | 81 |
| dNUDT5 (132) | N | 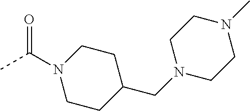 | 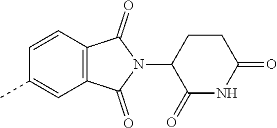 | 96 |
| 163 | N | 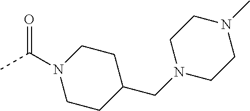 | 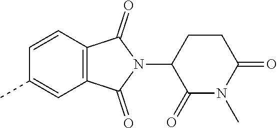 | 0 |
| 133 | N | 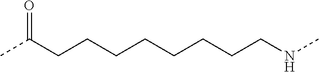 | 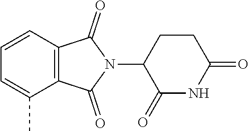 | 43 |
| 134 | N | 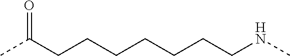 | 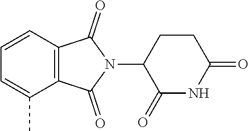 | 22 |
| 135 | N | 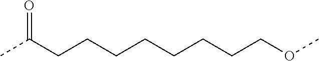 | 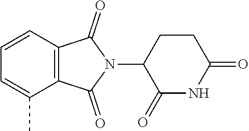 | 51 |
| 136 | N | 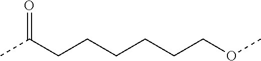 | 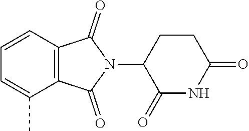 | NT |
| 137 | N | 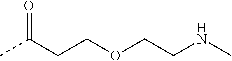 | 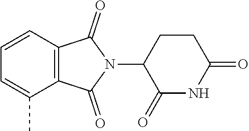 | 0 |
| 159 | N | 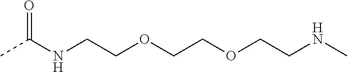 | 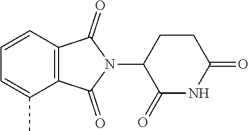 | 20 |

Compounds which are not of the invention are included for reference purposes.

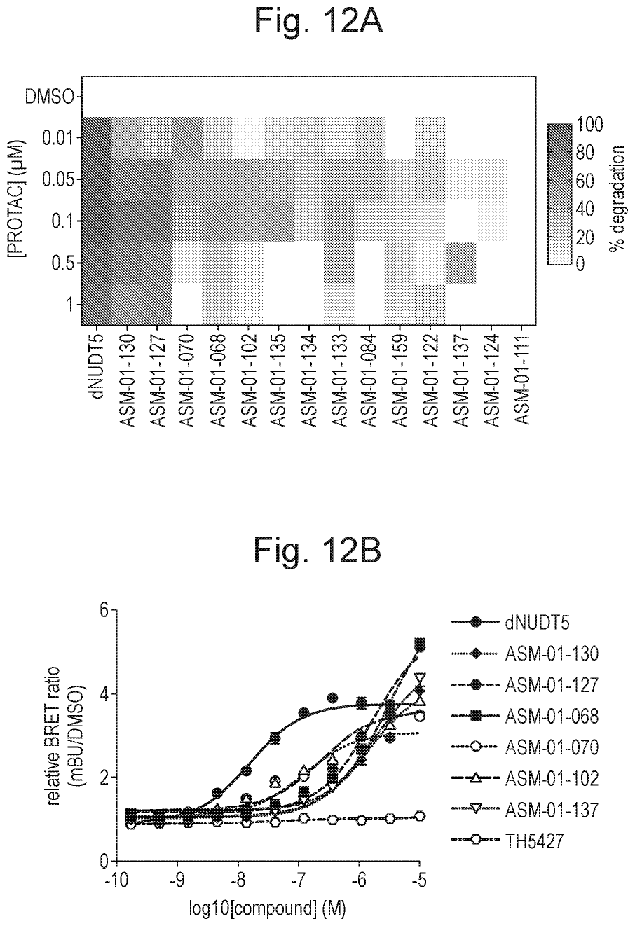

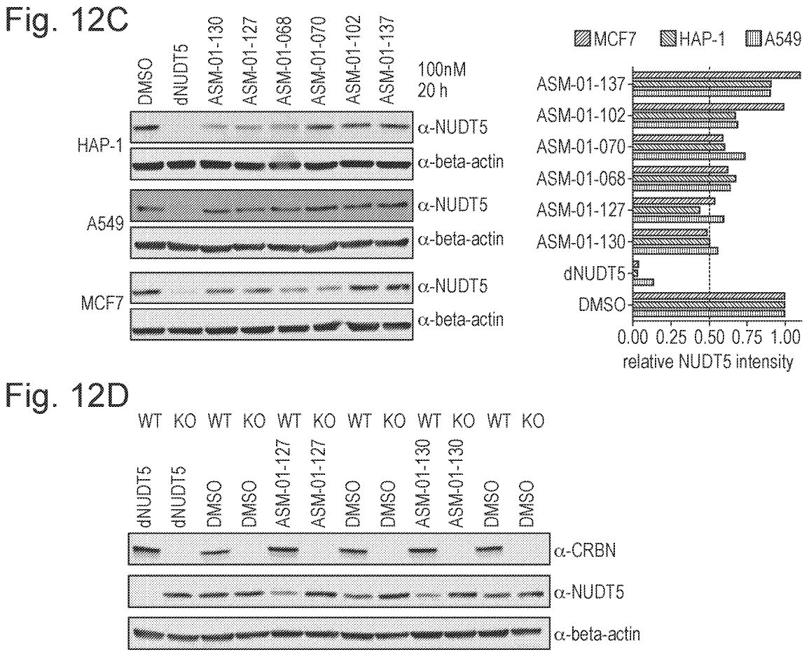

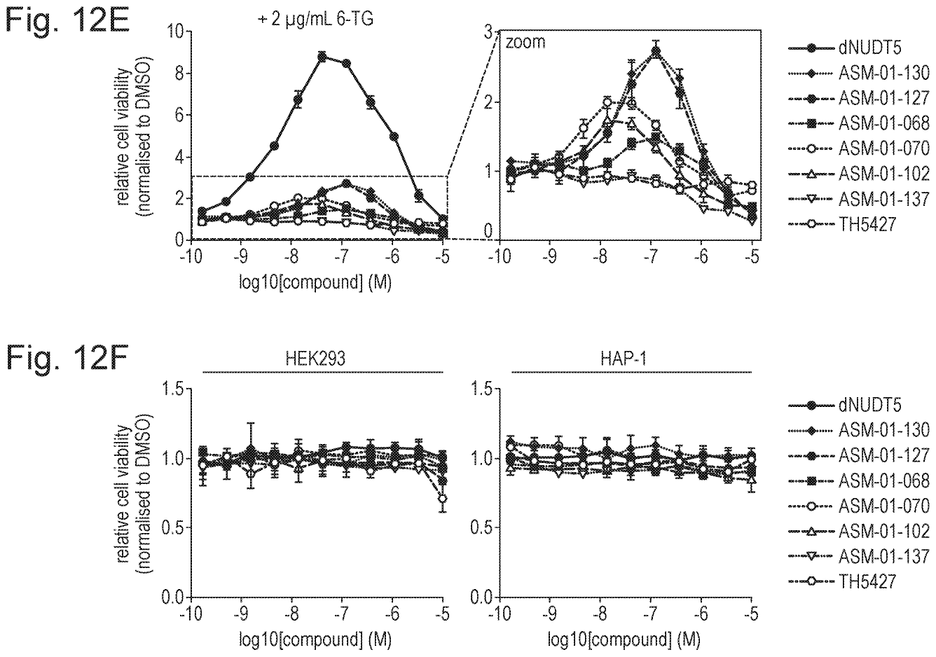

A large number of compounds including the above were synthesised. All these compounds were found to recruit NUDT5 in the nanomolar range but only a few of them proved to form the ternary complex between NUDT5 and the E3 ligase, CRBN. The results of a nanoBRET ternary complex formation assay for certain compounds is shown in FIG. 12B. The most promising degraders were then tested for degradation by Western Blot (FIG. 12 A). Interestingly, the carbon based degraders (e.g. 134) were either poor or inefficient NUDT5 degraders independent of the linker length. Only PEG or less flexible linkers were found to trigger a decrease in protein levels. A too short linker could not induce E3 ligase recruitment whereas longer linkers provided better candidates. This trend was observed with compounds 137 (1 PEG), 068 (2 PEG), 127 (3 PEG) and 130 (4 PEG). The longer PEG, the better degradation was achieved. When compared to compound 102, the one-missing carbon found in 070 was not compromising the degradation as both showed similar degradation efficiency. Not only the linker length played a role in inducing degradation. The linkage was also important as shown in compound 159 that bears a PEG linker and urea attachment point to the X group and compound 068 with a PEG linker and amide linkage. The first one could not induce degradation whereas the second one had a Dmax of 50%. This trend was also observed with 110, 070 and 071 which all had the same linker with CRBN ligand but different linkage positions. Only 070 induced the ternary complex and proved to be a reasonable NUDT5 degrader. Degradation activity is shown in FIG. 12A.

The best degraders were then tested in other different cell lines and proved to be efficient with similar degradation level at 100 nM (FIG. 12C). A CRBN knock-out experiment was also conducted and confirmed that dNUDT5 and degraders 127 and 130 are E3-ligase-dependant (FIG. 12D).

Further, to test the phenotypic effect of NUDT5 degradation, a cell viability assay was also conducted upon addition of 6-thioguanine (6-TG) (FIG. 12E). Previously, it was shown that genetic NUDT5 knockdown protected against 6-TG induced cytotoxicity. The present degraders showed a dose dependent rescue of HAP-1 cells in presence of 6-TG, which peaked at around 100 nM for all of them and decreased afterwards accounting for a potential Hook effect. As expected, TH5427 (corresponding to the X group carrying a hydrogen atom in place of a linker and Y group), and DMSO did not show a rescue of HAP-1 cells in the presence of 6-TG.

Further, the compounds were found not to significantly adversely affect cell viability. Results of cell viability tests after treatment with compounds of the invention are shown in FIG. 12F.

Activity of dNUDT5

### Results of Cell Assays

### Distinct MTHFD1 Enzymatic Activities Control a Switch Between Adenosine Dependency and Toxicity

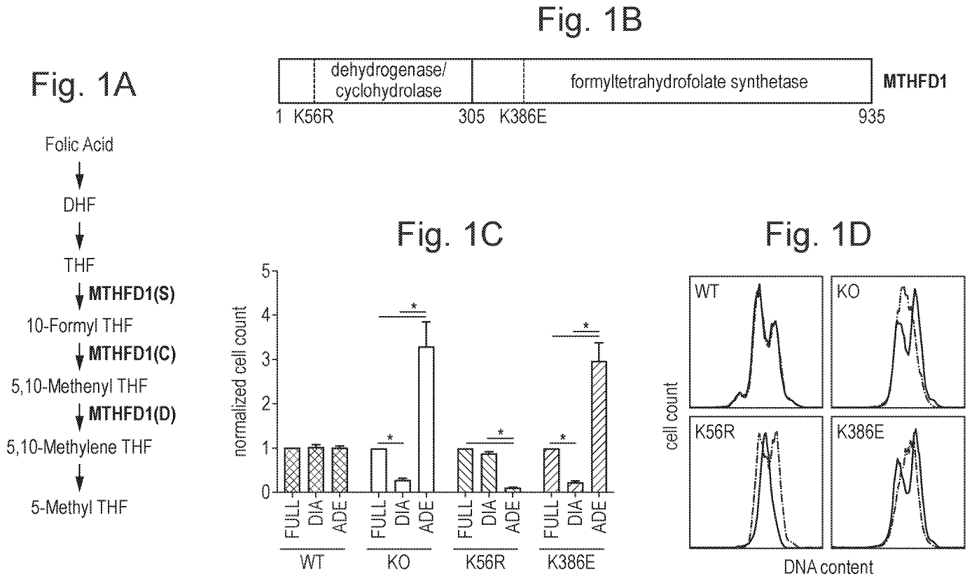

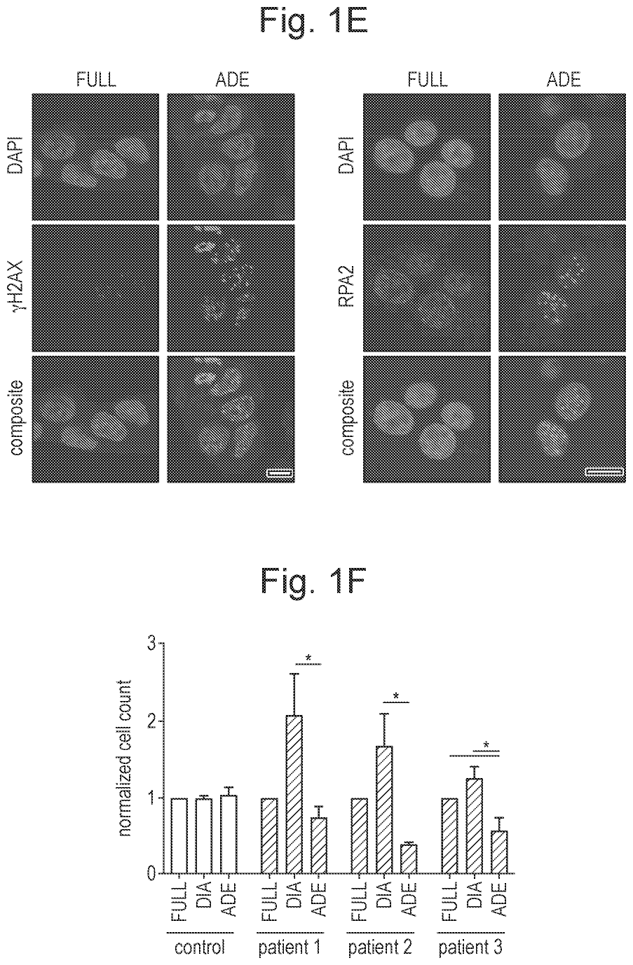

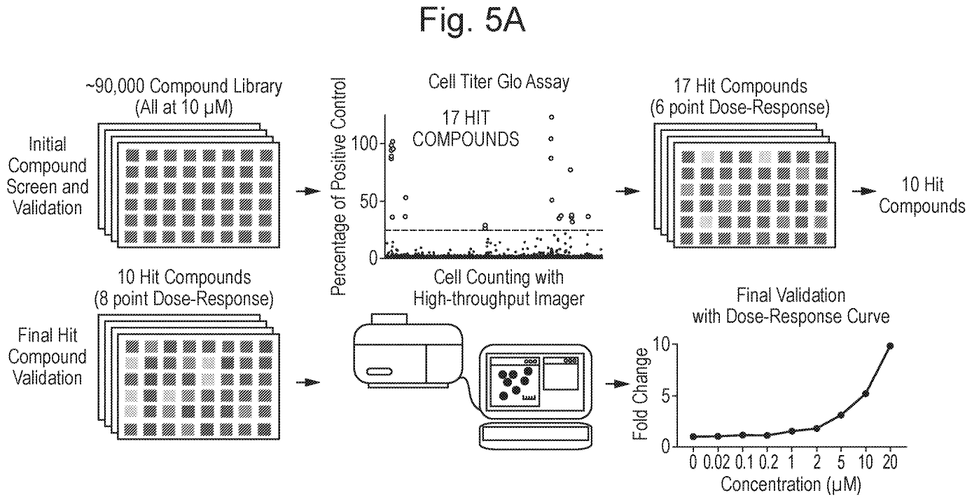

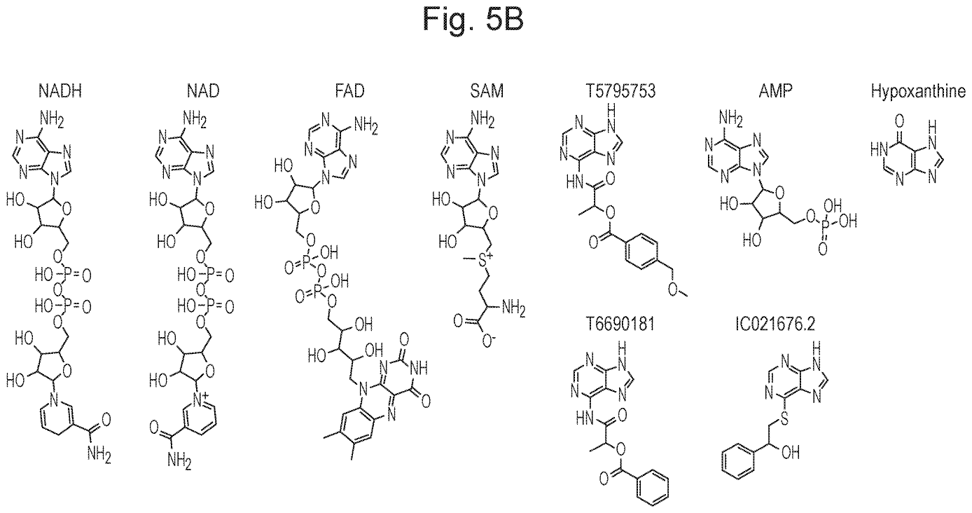

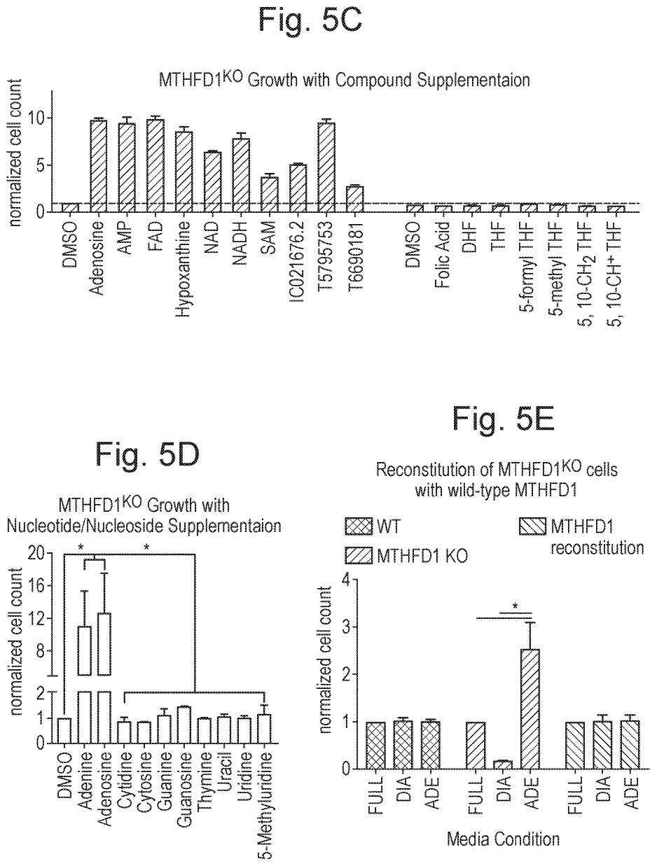

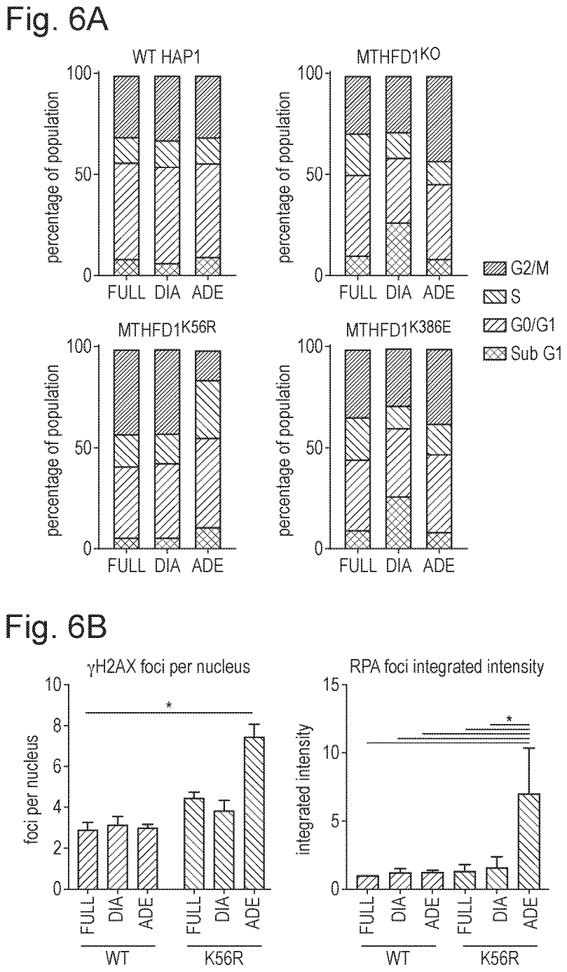

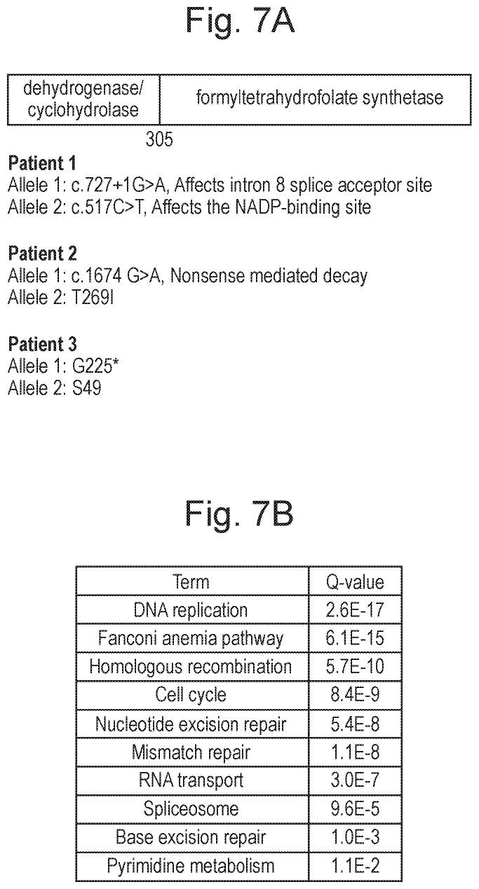

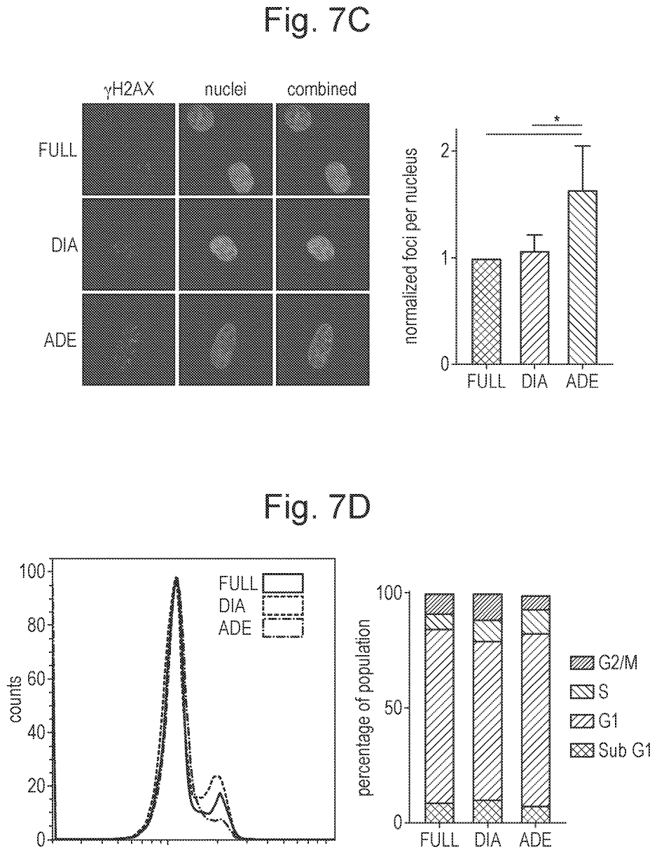

We engineered wild-type HAP1 cells (WT) and derived clonal cell lines that either contain a deletion in the MTHFD1 gene (MTHFD1^{KO}), or have been reconstituted with either the wild-type allele (MTHFD1^{res}) or point mutations in the synthetase (MTHFD1^{K386E}) or cyclohydrolase (MTHFD1^{K56R}) domains (FIG. 1B). We then tested the viability of these cell lines in normal media conditions and in media containing dialyzed serum lacking metabolites smaller than 10 kDa (FIG. 1C). MTHFD1^{KO} and MTHFD1^{K386E} cells were highly sensitive to serum dialysis, whereas the growth of WT and MTHFD1^{K56R} cells was unaffected in these conditions. To identify compounds that can counteract the detrimental effects of dialyzed serum on the growth of MTHFD1^{KO} cells, we performed a chemical screen of more than 90,000 structurally highly diverse small molecules (FIG. 5). We found that only adenine-containing compounds (e.g., NADH, NAD, FAD, SAM, AMP) or adenine precursors (e.g., hypoxanthine), but not other purines like guanines were able to rescue the growth of MTHFD1^{KO} cells (FIG. 5). Adenosine addition also enhanced the growth of synthetase mutant MTHFD1^{K386E} cells. However, in cyclohydrolase mutant MTHFD1^{K56R} cells we observed the diametrically opposite phenotype, as in that context addition of adenosine caused a strong antiproliferative response (FIG. 1C). These changes were also reflected in cell cycle analysis, where MTHFD1^{KO} and MTHFD1^{K386E} respond with adenosine-dependent G1/S arrest to dialyzed serum conditions, whereas MTHFD1^{K56R} cells arrest in G1/S following adenosine addition (FIG. 1D, FIG. 6A). Drastic increases in γH2A.X and RPA foci formation in these conditions (FIG. 1E, FIG. 6B) suggest that replication stress and DNA damage cause the phenotype. This effect was not specific to HAP1 cells, as we also observe adenosine toxicity in fibroblasts from MTHFD1 deficient patients (FIG. 1F, FIG. 7).

### Genome-Wide Genetic Screens Identify Modulators of MTHFD1-Mediated Adenosine Responses

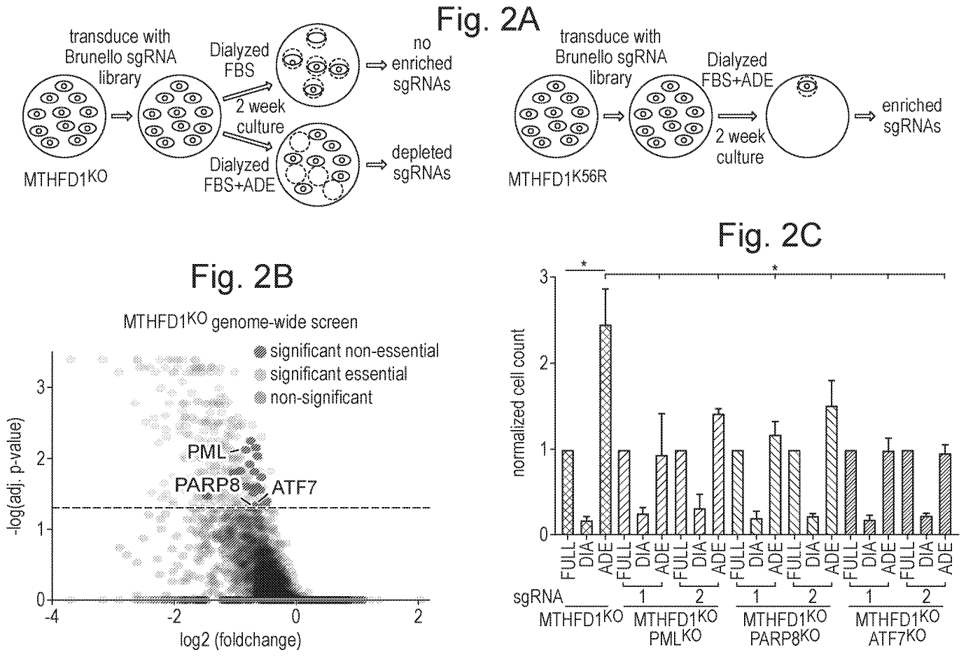

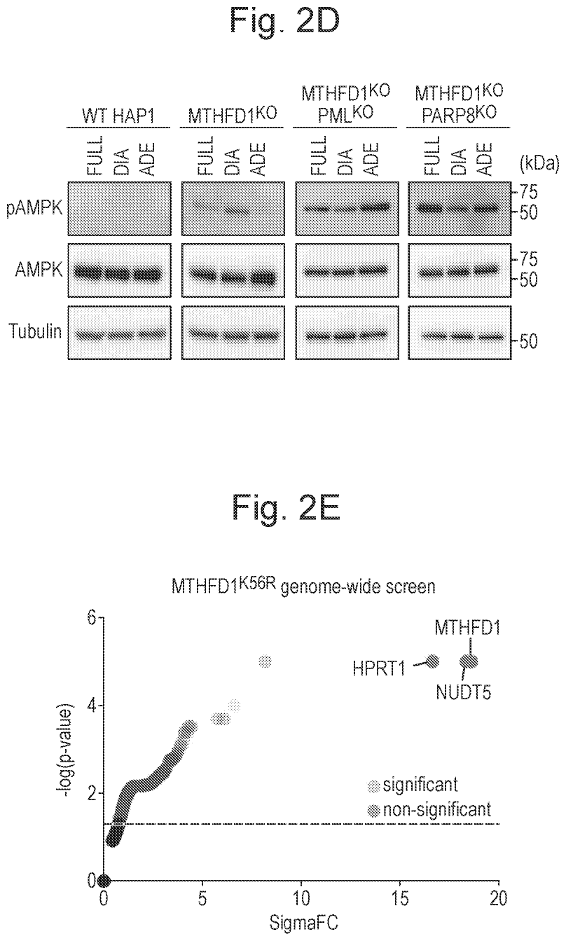

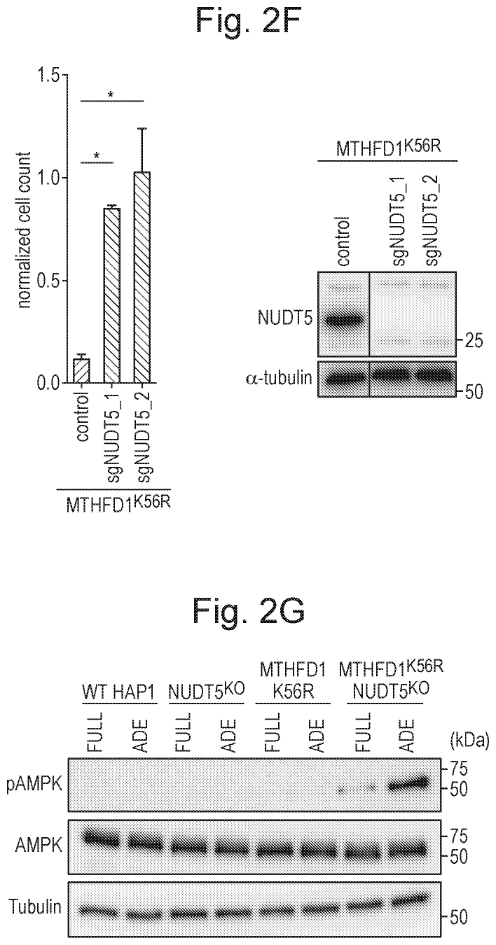

To probe the genetic dependencies caused by MTHFD1 mutations, we performed genome-wide genetic loss-of-function screens. We transduced HAP1 clones with the Brunello knockout library targeting 19,114 human genes with an average of four sgRNAs per gene. Following selection and growth for two weeks, we analyzed sgRNA abundance as a proxy for growth effects following knock-out of the respective target gene (FIG. 2A).

First, we transduced MTHFD1^{KO} cells and cultivated them for two weeks in medium containing dialyzed serum. We did not detect any significantly enriched sgRNAs in the few surviving cells in these toxic conditions (Data FIG. 5). This data indicates that no knock-out of a single non-essential gene can phenocopy the growth-restoring effect of adenosine addition, an observation that is consistent with the lack of any pharmacological pathway inhibitor being able to achieve this effect (FIG. 5).

In a second screen, we cultivated the same mutagenized MTHFD1^{KO} cells in media containing adenosine. Following 14-day growth, no sgRNAs were significantly enriched, suggesting that gene knock-outs cannot further enhance the growth promoting effects of adenosine. In contrast, sgRNAs for 290 target genes were significantly depleted in this population (FIG. 2B, Data FIG. 6). As expected, the majority of these genes are commonly essential and their knock-out is also detrimental to the proliferation of HAP1 WT cells in normal media conditions (22). However, 22 genes were selectively depleted in MTHFD1^{KO} cells grown with adenosine, among them genes coding for the PML nuclear body scaffold, the poly(ADP-ribose) polymerase PARP8, and the activating transcription factor ATF7. We confirmed that double knock-out in MTHFD1^{KO}PML^{KO}, MTHFD1^{KO}PARP8^{KO}, and MTHFD1^{KO}ATF7^{KO} cells blunted the growth-promoting effects of adenosine (FIG. 2C).

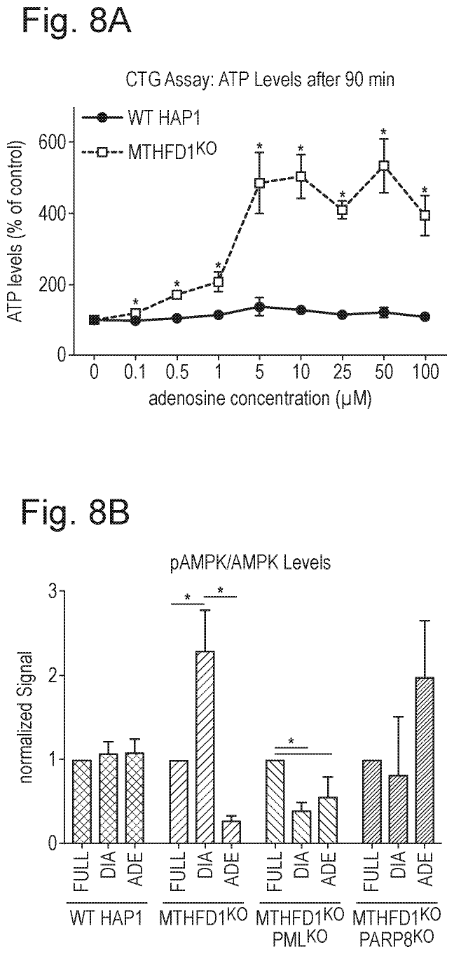

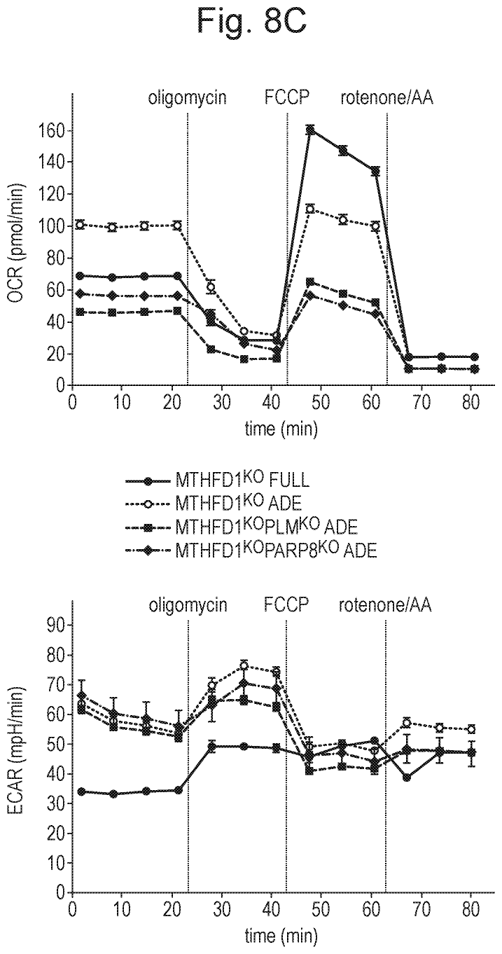

We reasoned that if adenosine was being metabolized in the MTHFD1^{KO} cells, some of the adenosine would be converted to ATP via the purine salvage pathway. Confirming our hypothesis, after a short 90-minute incubation with adenosine, we observed a significant dose-dependent increase in ATP levels in the MTHFD1^{KO} cell lines, whereas ATP levels stayed constant in the WT cell line (FIG. 8A). These data further suggest that the knockout of MTHFD1 led to a deficiency in ATP levels. Consistently we found that the central cellular energy sensor AMPK was activated in the MTHFD1^{KO} cells in standard medium, as measured by pAMPK immunoblotting (FIG. 2D, 8B). This activation was more pronounced when moving cells to media containing dialyzed serum, whereas adenosine addition completely countered AMPK activation. In MTHFD1^{KO}PML^{KO} and MTHFD1^{KO}PARP8^{KO} double knock-out cells, AMPK activation persisted despite adenosine addition (FIG. 2D, 8B), validating these genes as modulators of AMPK signaling. By performing bioenergetics studies, we find that the knockout of PML and PARP8 ablates adenosine-mediated, OXPHOS-based ATP production, leading to a continued ATP deficiency and chronic AMPK activation (FIG. 8C,D)

Finally, we also conducted a genome-wide genetic screen in MTHFD1^{K56R} with addition of an, in that context, toxic adenosine concentration. We discovered that the knock-out of three genes was able to strongly prevent adenosine toxicity: MTHFD1, HPRT1, and NUDT5 (FIG. 2E, Data FIG. 7). MTHFD1 served as a positive control, as a shift from the K56R mutation to the full knockout was expected to correlate with the transition from adenosine toxicity to adenosine dependence. HPRT1 is an enzyme in the purine salvage pathway that plays a key role in recycling purines (23, 24). We hypothesize that the HPRT1 knockout prevents adenosine-mediated toxicity by preventing functional utilization of exogenous adenosine in intracellular biochemical pathways. The third gene that can strongly prevent adenosine toxicity in MTHFD1^{K56R} cells, NUDT5, is a hydrolase involved in the catabolism of ADP-ribose and in nuclear ATP synthesis (25, 26). We validated that NUDT5 knock-out with two independent sgRNAs resulted in significantly enhanced proliferation in adenosine containing media compared to the parental MTHFD1^{K56R} line (FIG. 2F). Surprisingly, this increased growth occurs concomitant with an even stronger activation of AMPK (FIG. 2G).

A NUDT5 Scaffolding Function Rather than Enzymatic Activity is Essential for Modulating Adenosine Responses

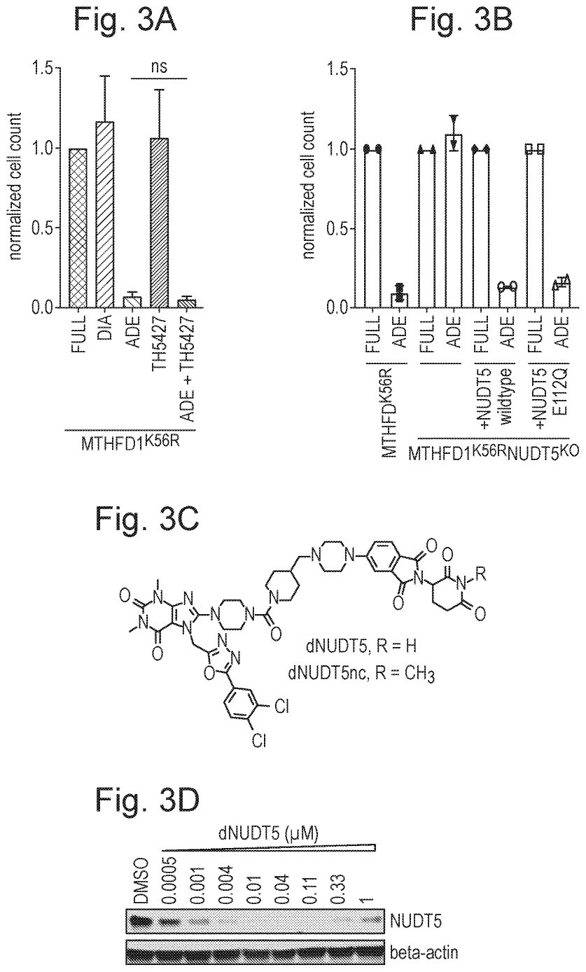

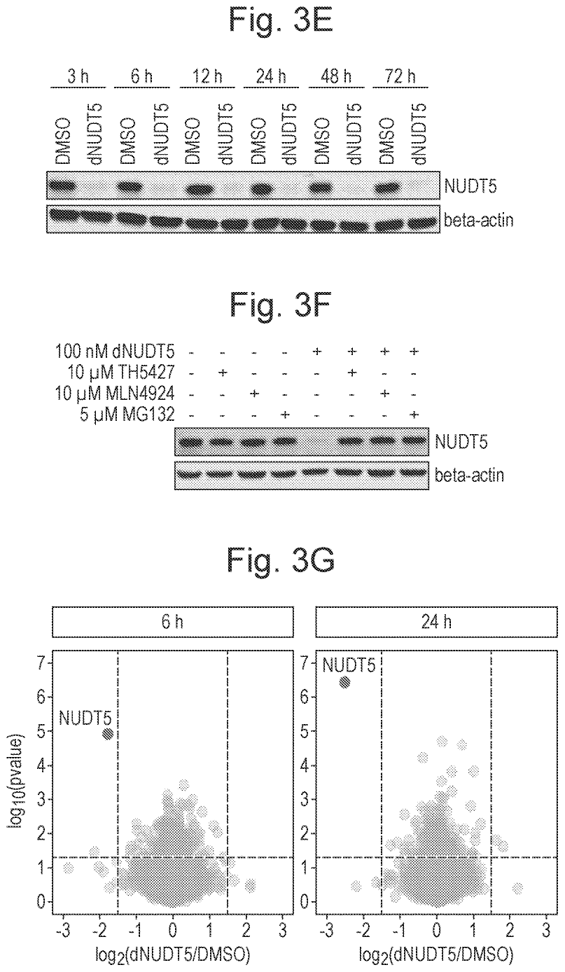

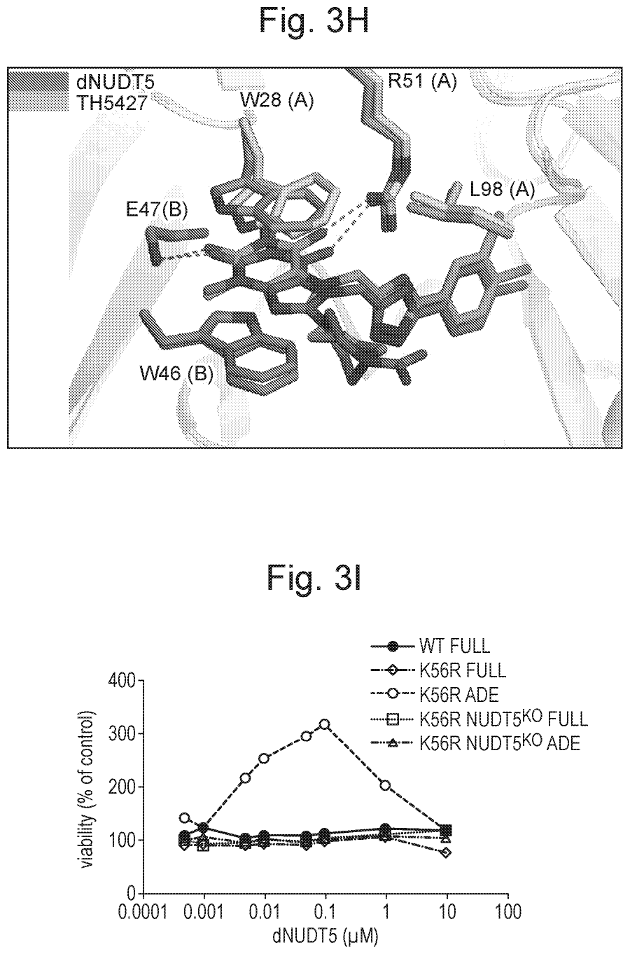

NUDT5 enzymatic activity has been previously linked to purine and adenine metabolism with recent reports suggesting the enzyme can generate ATP from ADP-ribose in the cell's nucleus (25). To assess whether the enzymatic activity of NUDT5 was essential for rescuing the viability of MTHFD1^{K56R} in adenosine-containing media, we first used a well-characterized chemical inhibitor of the enzyme's activity, TH5427 (FIG. 3A)(27). Even at doses more than 1000× higher than its biochemical IC₅₀ of 29 nM, TH5427 treatment did not affect the viability of MTHFD1^{K56R} cells in adenosine-containing media. To assess whether this effect was due to compromised compound uptake or activity in HAP1 cells, we next reconstituted MTHFD1^{K56R}NUDT5^{KO} cells with either wildtype NUDT5 or a catalytically inactive NUDT5(E112Q) mutant. Interestingly, both variants of the protein resensitized MTHFD1^{K56R}NUDT5^{KO} cells to adenosine, strongly suggesting that the enzymatic function of NUDT5 is dispensable for adenosine-mediated toxicity (FIG. 3B).

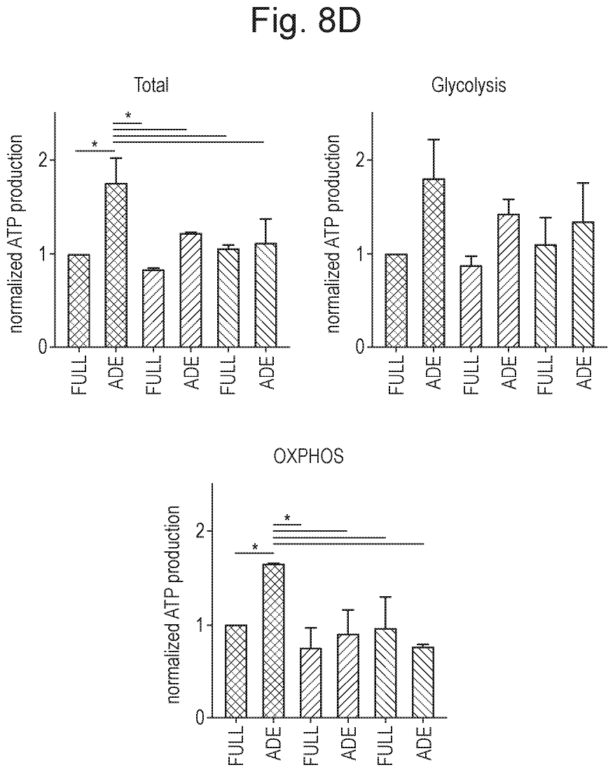

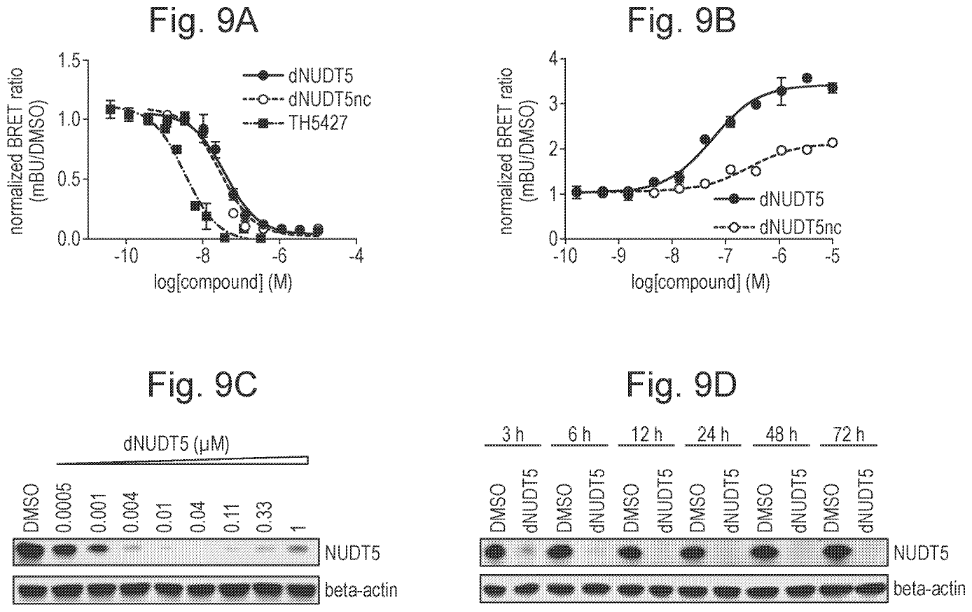

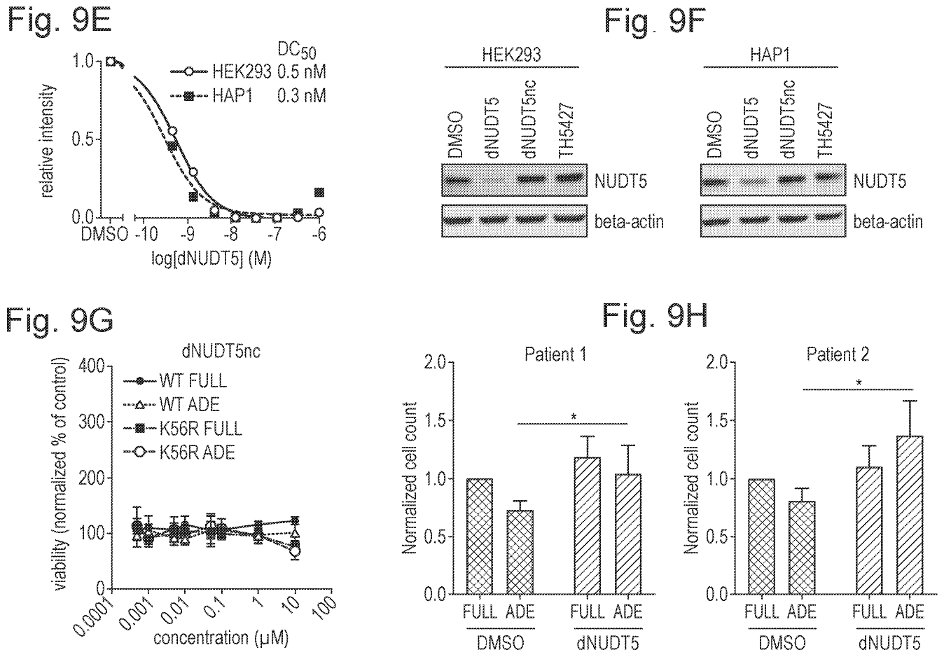

To confirm that the physical presence of NUDT5 protein, rather than its enzymatic function or any downstream cellular adaptation, would explain the effect observed with the genetic knock-out, we decided to develop a small molecule tool compound for the acute chemical degradation of NUDT5. The resulting compound, dNUDT5 (FIG. 3C), is a PROTAC that induces proximity between NUDT5 and the cereblon (CRBN) E3 ligase complex. We also obtained a corresponding negative control, dNUDT5nc, by N-methylation of the imide ring to attenuate CRBN binding. Both dNUDT5 and dNUDT5nc engaged their cognate target NUDT5 in live cells as shown by NanoBRET assays (FIG. 9A). However, only dNUDT5 was able to induce proficient ternary complex formation between NUDT5 and CRBN (FIG. 9B) This translated into strong NUDT5 degradation in HEK293 and HAP-1 cells in a concentration- and time-dependent manner (DC₅₀=0.5 nM and 0.3 nM, respectively, FIG. 3D, E, FIG. 9C-F). In line with the need for concomitant ligand binding of both NUDT5 and CRBN for efficient ternary complex formation, we observed that degradation efficiency is declining at concentrations beyond 300 nM, a hook effect commonly observed with PROTACs. Rescue experiments confirmed that NUDT5 degradation by dNUDT5 was E3 ligase- and proteasome-dependent (FIG. 3F). Next, we performed mass spectrometry analyses of HAP-1 cells treated with dNUDT5. These data indicate dNUDT5 is highly specific for NUDT5 with no other protein being degraded in this context (FIG. 3G). A crystal structure of dNUDT5 bound to NUDT5 confirmed that the binding mode was not significantly altered compared to the parental NUDT5 ligand (FIG. 3H), consistent with the observation that TH5427 is able to prevent NUDT5 degradation by dNUDT5 (FIG. 3F).

After extensively characterizing our degrader, we treated WT and MTHFD1^{K56R} cells with dNUDT5. While the compound did not affect the viability of WT cells, it prevented adenosine toxicity in MTHFD1^{K56R} with dose-responsiveness perfectly matching NUDT5 degradation, with maximum rescue of cell viability at 100 nM concentration (FIG. 3I). As expected, the negative control compound dNUDT5nc did not cause any effects in these assays. (FIG. 9G). The availability of a chemical tool for rapid NUDT5 degradation further enabled us to test the effect on adenosine toxicity in cells derived from MTHFD1 deficiency patients. Matching the MTHFD1^{K56R} cell data, we show that addition of dNUDT5 also reverses adenosine-mediated toxicity, highlighting the potential role of NUDT5 in folate-related disorders (FIG. 9H).

### NUDT5 Controls the Repression of De Novo Purine Biosynthesis in Response to Adenosine Addition

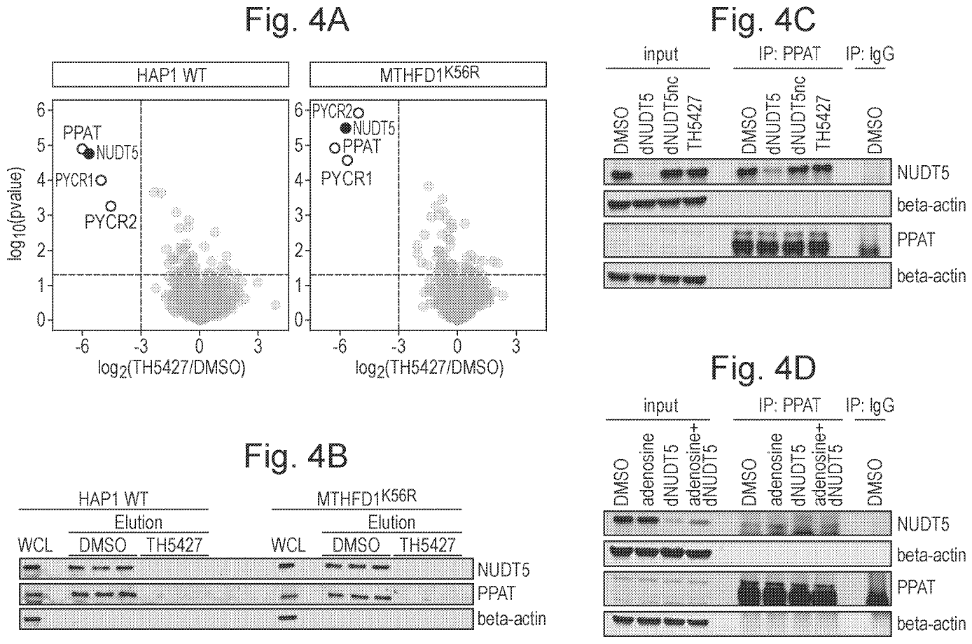

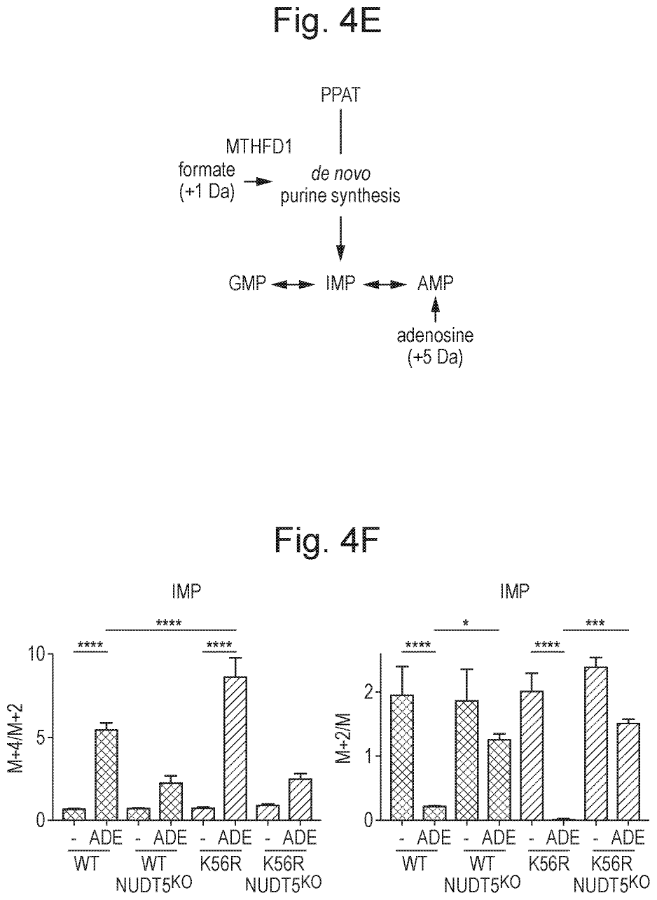

To identify NUDT5 binding partners that may be regulating adenosine-mediated toxicity, we performed chemoproteomics experiments (28) to investigate binders of the NUDT5 inhibitor TH5427 (FIG. 4A). Interestingly, we noted that in addition to NUDT5, phosphoribosyl pyrophosphate amidotransferase (PPAT), which catalyzes the first step of the de novo purine nucleotide biosynthetic pathway, was co-purified with NUDT5. We confirmed this interaction and showed that both proteins can be competed with an excess of non-immobilized TH5427 (FIG. 4B). Previous large-scale unbiased protein-protein interaction studies have also suggested PPAT as an interactor of NUDT5 (29), indicating that this interaction is not compound-mediated. Consistently, we find that the NUDT5-PPAT interaction is also observed with pull-down of endogenous PPAT (FIG. 4C). Treatment of cells with dNUDT5, but not the inactive degrader or catalytic inhibitor TH5427, strongly reduced the amount of NUDT5 interacting with PPAT and NUDT5 (FIG. 4C). Adenosine treatment appears to enhance the interaction between NUDT5 and PPAT, and partially interfere with NUDT5 degradation by dNUDT5, possibly by occupying the same NUDT5 binding pocket (FIG. 4D).

Given that PPAT is the first enzyme in the purine de novo synthesis pathway, we assessed whether loss of NUDT5 affected the flux through this pathway. We treated cells with isotope labeled formate (+1 Da) that is incorporated into de novo synthesized purines via 10-CHO-THF produced by the MTHFD1 synthetase activity (FIG. 4E). To assess incorporation of externally added adenosine into different metabolites, we used isotope labeled adenosine (+5 Da). As every de novo-synthesized purine contains two formate-derived carbons, we analyzed the relative abundance of the M+2 peak for different purines, including AMP, GTP and IMP.

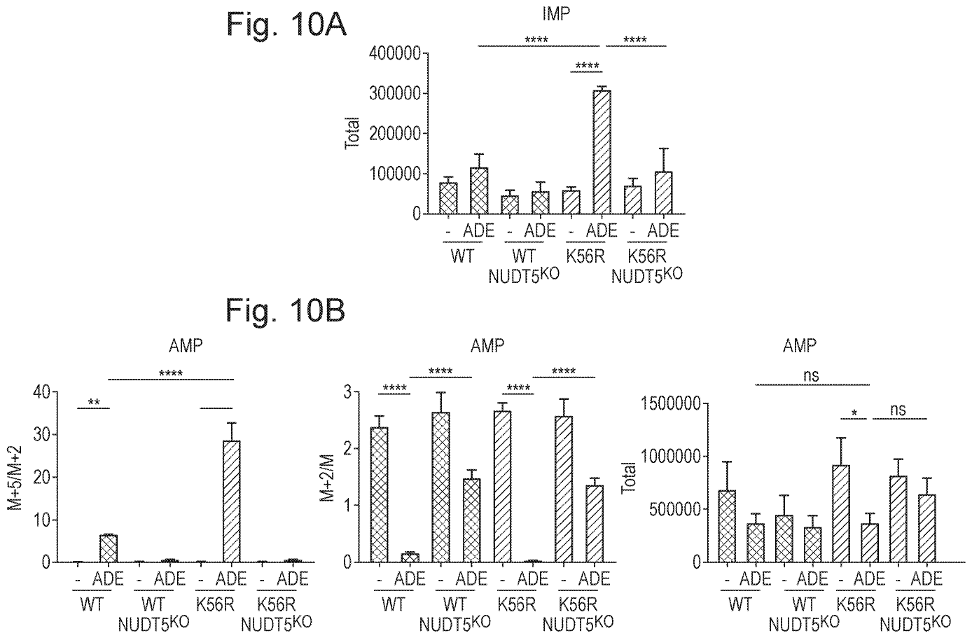

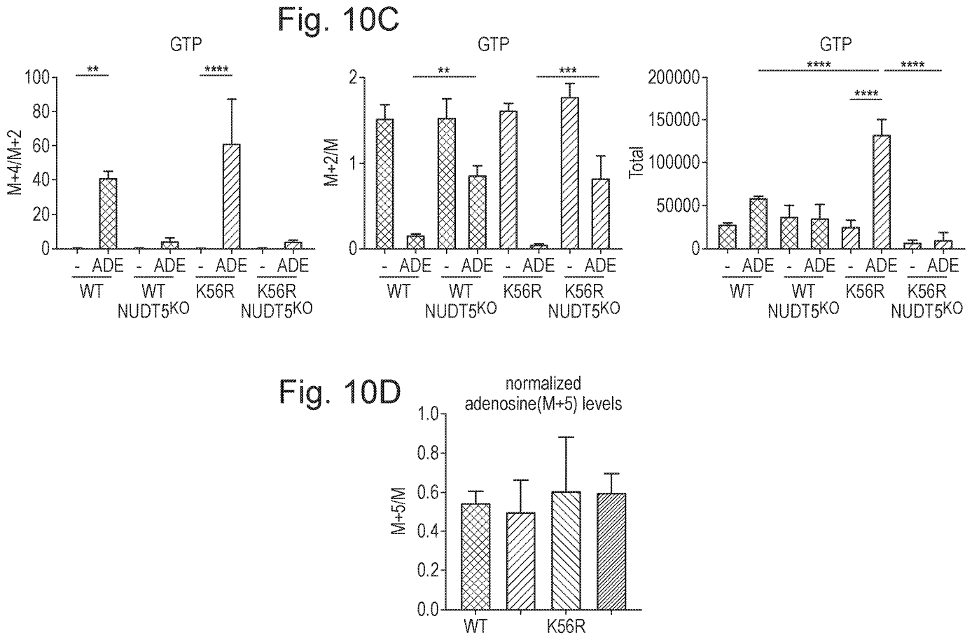

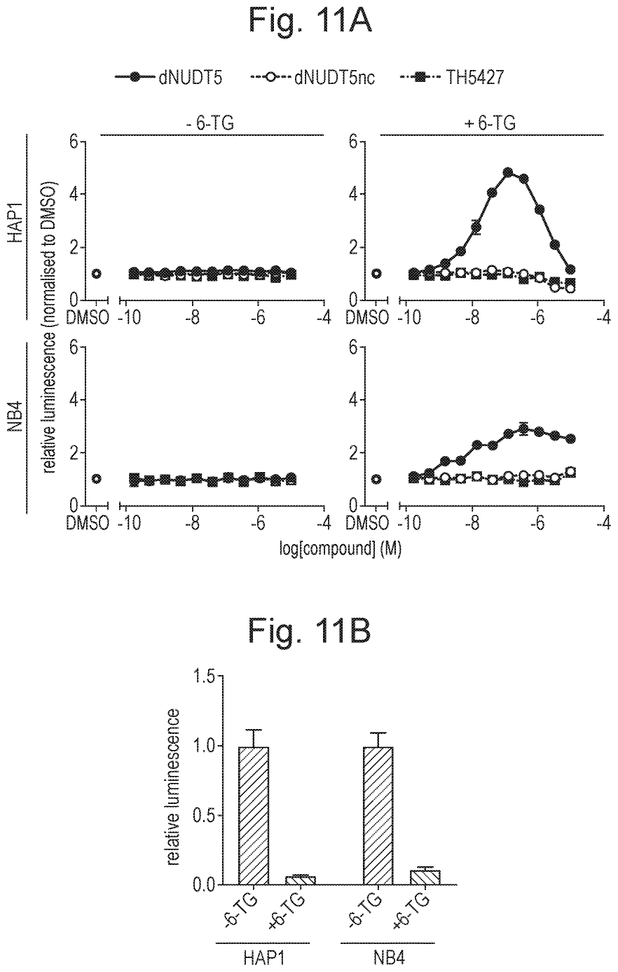

For all metabolites, adenosine addition resulted in a strong repression of de novo synthesis in both WT and MTHFD1^{K56R} cells (FIG. 4F, FIG. 10A-C). However, NUDT5 knock-out prevented this repression and resulted in a drastically elevated proportion of de novo-derived AMP, GTP and IMP as well as a significant decrease in total purine levels (FIG. 10A-C). This observation is intriguing given the previously published role of NUDT5 in regulating 6-thioguanine (6-TG) toxicity (30). The increased de novo purine synthesis coupled with decreased purine levels possibly suggests that the knockout of NUDT5 leads to decreased 6-TG by diluting out the effect of 6-TG metabolized through the purine salvage pathway. Indeed, we found that only dNUDT5 was able to rescue 6-TG toxicity whilst neither the negative control nor TH5427 gave any significant effects, again strongly suggesting a critical scaffolding role of NUDT5 in the purine salvage pathway (FIG. 11).

### Discussion

By initially characterizing the pharmacological and genetic dependencies of MTHFD1 mutations, we discovered that the NUDIX hydrolase NUDT5 acts as a novel regulator of de novo purine synthesis. Specifically, we describe an orthogonal scaffolding role of NUDT5 in repressing purine synthesis. The knockout or degradation of NUDT5 leads to increased flux through de novo purine synthesis, suppression of the purine salvage pathway and decreased total intracellular purine levels. These findings thereby significantly enhance our understanding of the biology of PPAT in regulating the first step in de novo purine biosynthesis. Historical results have shown that allosteric regulation of PPAT by adenosine addition causes the formation of a higher molecular weight complex with reduced enzymatic activity (31). Here, we find the physical interaction of NUDT5 with PPAT underlies the purine salvage-mediated reduction of flux through de novo synthesis. Cell death caused by adenosine in MTHFD1 mutant conditions appears to be caused by nucleotide imbalance and resulting DNA damage, consistent with earlier findings of altered adenine to guanine nucleotide balance with modulation of de novo synthesis (32). Our findings can have translational applications, as indicated by modulation of the toxicity of the approved cancer drug 6-TG with dNUDT5, as well as the finding that degradation of NUDT5 in MTHFD1-deficient patient-derived cells reverses the cell arrest phenotype brought on by adenosine supplementation.

### Materials and Methods

### Cell Culture and Transfection

HAP1 (KBM7-derived, Horizon Discovery, C859), and HAP1 MTHFD1^{KO}, MTHFD1^{K56R}, and MTHFD1^{K386E} cell lines were cultured in Iscove's Modified Dulbecco's Medium (IMDM, Sigma), supplemented with 10% Fetal Bovine Serum (FBS, Sigma) and 1% Penicillin/Streptomycin (P/S, Sigma).

Patient-derived fibroblast and control fibroblast lines were cultured in Minimum Essential Medium alpha media (MEMα, no nucleosides, Gibco), supplemented with 10% FBS and 1% P/S. These patient-derived fibroblast lines were previously characterized in Burda et al. 2015. For the experiments using dialyzed FBS, the dialyzed FBS was purchased from Gibco and added to IMDM at a final concentration of 10%. All the cell lines were incubated in 5% CO₂ atmosphere at 37° C.

HAP1 MTHFD1^{KO} and MTHFD1^{K56R} cell lines were transfected with Turbofectin 8.0 (Origene) according to manufacturer's instructions.

### Western Blot Analysis

For Western blots, proteins were separated on polyacrylamide gels with SDS running buffer (50 mM Tris, 380 mM Glycine, 7 mM SDS) and transferred to PVDF membranes. All membranes were then blocked with blocking buffer (5% w/v milk powder, BioRad) in TBST (50 mM Tris (tris(hydroxymethylaminomethane), 150 mM NaCl, 0.05% (v/v) Tween 20, adjusted to pH 7.6) Proteins were probed with antibodies against phospho-AMPK (50081S, 1:2000, Cell Signaling Technologies), AMPK (5832S, 1:2000, Cell Signaling Technologies), PML (ab179466, 1:1000, abeam), PARP8 (NBP213733, 1:1000, Novus Biologicals), ATF2/7 (82870, 1:1000, Cell Signaling Technologies), alpha-Tubulin (ab7291, 1:1000, Abeam), phospho-UBTF (ab182583, 1:1000, abeam), UBTF (HPA006385, 1:1000, Sigma), RBM38 (AV40862, 1:1000, Sigma), or NUDT5 (ab129172, 1:1000, abeam), detected by HRP (horse-radish peroxidase) conjugated donkey anti-rabbit IgG antibody (Pierce) or donkey anti-mouse antibody IgG antibody (Pierce) and visualized with Clarity ECL Western Blotting substrate (BioRad), according to provided protocol.

For detecting TH5427 interactors and the PPAT-NUDT5 interaction, protein samples were prepared using RIPA buffer containing benzonase (E1014-25KU, Sigma-Aldrich). Protein concentration was measured by DC assay (U.S. Pat. Nos. 5,000,113, 5,000,114, 5,000,115, Biorad). 30 μg protein out of each sample was separated on a SDS-page gel (3450125, Biorad) and transferred to a nitrocellulose membrane (10600014, Sigma-Aldrich) at 0.4 A for 1 h. The membranes were blocked by 5% BLOT-QuickBlocker in PBS-T at room temperature for 1 h (786-011, Bioscience) and incubated with primary antibodies at 4° C. overnight. Then, fluorophor conjugated secondary antibodies diluted 1:500 in 2×BLOT-QuickBlocker in PBS-T were added onto the membrane for 1 h at room temperature. Protein bands were visualized by a Odyssey® DLx Imaging System (Licor). The following primary antibodies were used: NUDT5 (ab129172, Abeam, 1:1000, anti-rabbit) or NUDT5 (sc-398644, 1:200, anti-mouse), β-actin (sc-69879, Santa Cruz Biotechnology, 1:200) and PPAT (15401-1-AP, Proteintech, 1:1000). The following secondary antibodies were used: Alexa Fluor 750 goat anti-mouse IgG (A21037, Life technologies) and goat anti-rabbit IgG (H+L) cross-adsorbed secondary Antibody, Alexa Fluor 647 (A21244, Life technologies).

### Single Metabolite Supplementation Screen

Nucleotide metabolites (adenine, adenosine, guanine, guanosine, cytidine, cytosine, uridine, uracil, thymine, 5-methyluridine) were purchased from Sigma and dissolved in DMSO at a concentration of 50 mM. deoxynucleotides (dATP, dCTP, dTTP, dGTP). Folate metabolites (Folic Acid from Sigma, 5-Formyl tetrahydrofolic acid, 5-methyltetrahydrofolic acid, 5,10-Methenyl tetrahydrofolic acid, T,10-methylene tetrahydrofolic acid, tetrahydrofolic acid, and dihydrofolic acid purchased from Schircks Laboratories) were dissolved in DMSO at a concentration of 40 mM. For the AMPK activity modulation studies, A-769662 (se-203790, Santa Cruz Biotechnology) and MK8722 (HY-111363, MedChemExpress) were dissolved in DMSO to create stock solutions of 100 mM and 20 mM, respectively.

MTHFD1^{KO} cells in dialyzed FBS media were treated with a single nucleotide or folic acid metabolite for 72 hours. Cells were then stained with Hoechst, imaged, and counted using an Operetta (Perkin Elmer). For the AMPK activator studies, cells were stained with Hoechst, imaged, and counted using an Opera (Perkin Elmer). Similarly, MTHFD1^{K56R} cells in dialyzed FBS media were treated with adenosine for 72 hours. Cells were then stained with Hoechst, imaged, and counted using an Operetta (Perkin Elmer).

### High Throughput Compound Screen

MTHFD1^{KO} cells (F5 clone) in dialyzed FBS media were treated with the CeMM compound library (89,228 chemically diverse compounds). Both cell number, using Hoechst staining, and CellTiter-Glo (Promega), an indicator of metabolically active cells, was both used as a readout, depending on the stage of the screen.

Briefly, the screening was divided into the three parts (i) primary screening, (ii) follow-up and (iii) validation. During the primary screen, MTHFD1^{KO} cells were treated with 10 μM of every compound and CellTiter-Glo (Promega) was used to assess their ability to increase cell growth after 48 hours. From this primary screening, 17 compounds were selected as hits and rescreened in the follow-up part, in which the MTHFD1^{KO} cells were treated in a six-point dose response for 72 hours to discard any false positives. Finally, in the validation part, MTHFD1^{KO} cells were treated with 10 compounds in an eight-point dose response curve and the cell numbers were counted by staining with Hoechst, imaging and counting nuclei using the Opera Phenix (Perkin Elmer) and Harmony software (Perkin Elmer).

Compounds were transferred on 384-well plates using an acoustic liquid handler (Echo 550, Labcyte/Beckman Coulter) and 1,000 cells per well were dispensed on top of the drugs using a dispenser (Multidrop, Thermo Fisher Scientific) for a total of 50 l/well. ATP levels was measured after 48 h using CellTiter-Glo (Promega) in a multilabel plate reader (EnVision, Pekin Elmer). Signal was then normalized to DMSO and adenosine control wells included on each. plate.

### RNA-Seq and GO Term Enrichment Analysis

WT HAP1 and MTHFD1^{KO} cells were incubated in either dialyzed FBS media supplemented with 50 μM adenosine or an equivalent volume of DMSO. After incubating for the indicated time (6 h, 24 h, and 48 h), the RNA was extracted using the RNeasy Mini kit (Qiagen). For the patient cells, the cells were incubated in FULL, DIA, or ADE media for 24 h and then the RNA was extracted using the RNeasy Mini kit (Qiagen). RNA was sequenced by the Biomedical Sequencing Facility at CeMM using the Illumina HiSeq3000/4000 platform and the 50-bp single-end configuration. Reads were aligned with TopHat. Cufflinks was used to assemble aligned RNA-Seq reads into transcripts, estimate their abundances, and test for significant differential expression. Genes were significant if the calculated q-value was less than 0.05. RNAseq data is stored with GEO study accession number GSE201334.

To generate the Principal Component Analysis (PCA) plot, we used a custom script, where expression data for each experimental condition were first averaged across all samples. Transcripts with zero expression observed across all conditions were subsequently removed. PCA was performed using stats::prcomp function in R on centered and scaled data. The resulting two largest principal components were plotted.

For the GO term enrichment analysis, up-regulated and down-regulated proteins were defined based on log(fold change) for each experimental condition. The enrichment analysis for resulting list of proteins was performed using enrichr API (https://maayanlab.cloud/Enrichr/) (Chen et al., 2013; Kuleshov et al., 2016) through ‘enricher’ package in R (https://cran.r-project.org/web/packages/enrichR/index.html) with “GO_Biological_Process_2018” library.

Significantly enriched processes (FDR-corrected p-value <0.05) for down-regulated proteins at 48 h for MTHFD1^{KO} cells were represented in the radar plot. The plot was produced using R package ‘ggradar’ (https://github.com/ricardo-bion/ggradar). The value represented on the axes show combined enrichment score (c), where c=z log(p) when p is the Fisher's exact test p-value and z is the z-score representing deviation from expected rank as described in Chen et al. 2013.

### Brunello CRISPR KO Library Amplification and Virus Production

Human Brunello CRISPR knockout pooled library (Addgene #73178, a gift from David Root and John Doench) was electroporated into Endura Electrocompetent (Biocat) cells using the manufacturer's suggested parameters and recovered in 1 ml of recovery media. After a recovery period of 1 h, the cells were plated onto 24.5 cm² bioassay agar plates with ampicillin. Plates were then grown at 30° C. for 16 hours. Colonies were harvested from the plates and DNA was collected using a Maxi-prep kit (Qiagen).

HEK293T cells were then transfected with the maxi-prepped plasmid DNA. 7.5 million HEK293 Ts were seeded in a 15 cm dish in 25 ml of Dulbecco's Modified Eagle's Medium (DMEM) supplemented 10% FBS and 1% P/S. The next day, the HEK293 Ts were transfected using a master mix containing pMD2G (Addgene plasmid #12259, a gift from Didier Trono) and psPAX2 (Addgene #12260, a gift from Didier Trono) packaging plasmids and PEI transfection reagent (1 mg/ml, Sigma). For each plate, 11.65 μg of library plasmid, 5.83 μg of MD2G and 8.74 μg of PSPAX2 was used. First, a master mix was made which contained the MD2G, PAX2 and plasmid library in 888 μl of serum- and antibiotic-free DMEM media. 145 μl of PEI was added to the mixture and the mixture was vortexed for 10 seconds. The mixture was incubated for 15 min at room temperature to allow for complex formation. During this 15 min incubation time period, the media was aspirated from the 15 cm dish containing the HEK293T cells and 16 ml of DMEM (with FBS and P/S) was added. After the 15 min incubation, 2300 μl of DMEM (with 10% FBS and 1% P/S) was added to the complexes and pipetted up and down to mix. The complex mixture was added to the plate and the dish was gently swirled to distribute the complexes. Supernatant was harvested after 48 hours, filtered through a 0.45 um filter and aliquots frozen at −80° C.

Virus was then titrated in MTHFD1^{KO} cells. Briefly, MTHFD1^{KO} cells were treated with various dilutions of virus (1:4 to 1:200 of the total volume in the well) and Polybrene (5 μg/ml, Santa Cruz Biotechnology) in IMDM (supplemented with 10% FBS and 1% P/S) for 48 hours. The media was then aspirated and replaced with media containing 1 μg/ml Puromycin (Sigma) for 48 hours. After 48 hours, the dead cells were washed away and the live cells were counted. The dilution of virus that led to 25-35% cell survival (corresponding to a MOI of ~1) as compared to a no-puromycin control was used for further experiments. For the K56R genetic screen, the virus was titrated in the same manner with the MTHFD1^{K56}R cells.

### Genome-Wide CRISPR Screens

The genome-wide CRISPR screen was performed as described by Joung et al (Joung et al., 2017). Briefly, MTHFD1^{KO} cells were transduced with the previously titrated virus in media containing 5ug/ml Polybrene. A sufficient number of cells/dishes were treated to achieve more than 500× coverage of the Brunello plasmid library. After 48 hours, the media was aspirated and fresh media supplemented with lug/ml Puromycin was added. After another 48 hours, the cells were either collected (Control condition) or changed to a different media condition. For the dialyzed condition (DIA), the media composition was IMDM with 10% dialyzed FBS and 1% P/S. For the adenosine condition (ADE), the media composition was IMDM with 10% dialyzed FBS, 1% P/S, and 50 M adenosine. For both conditions, media was replaced every 3-4 days for a total of 14 days. After that, the cells were collected and the genomic DNA was extracted using a DNeasy Blood and Tissue Kit. Guide RNA sequences were amplified and attached with adapters using the primers from Joung et al (Joung et al., 2017). Product was bead purified (A63880, Beckman Coulter) and sequenced. Sequenced guides were analyzed using CRISPRAnalyzeR (excluding guides with read counts less than 20 for a more robust analysis and potential hits were calculated using the MaGECK algorithm(Li et al., 2014) (adjusted p-value <0.05). These potential hits were cross-referenced with the previously published list of HAP1 essential genes (Blomen et al., 2015) and all essential genes were excluded. To determine the gene hits in the ADE condition, the non-essential genes hits in the ADE and DIA were compared.

For the K56R genome-wide screen, the cells were transduced in the same manner as the MTHFD1^{KO} cells. The transduced cells were then subjected to adenosine supplemented (50 μM) media for 2 weeks. After that, the cells were collected and the genomic DNA was extracted using a DNeasy Blood and Tissue Kit. Guide RNA sequences were amplified and attached with adapters using primers adapted from Mayor-Ruiz et al (Mayor-Ruiz et al., 2019). Product was gel purified and sequenced. Sequenced guides were analyzed with PinAPL-Py online tool (Spahn et al., 2017).

### Generation of MTHFD1 Mutants

MTHFD1 cDNA was ordered from Genscript and cloned into lentiviral compatible vector (Addgene plasmid #17448, a kind gift from Eric Campeau and Paul Kaufman). K56R and K386E mutants were created using the Q5 site-directed mutagenesis kit according to manufacturer's instructions. The constructs were then packaged as described earlier. MTHFD1^{KO} cells were then transduced with a single construct and single clones were generated. For the genome-wide screen, the K56R mutant was also cloned into the lentiviral backbone with neomycin, and not puromycin, resistance (Addgene plasmid #17447, a kind gift from Eric Campeau and Paul Kaufman).

### Generation of CRISPR Screen Hit KO Cell Lines

To generate single-gene KOs to validate the MTHFD1^{KO} screen, guide RNA (gRNA) target sequences were chosen from the Brunello library sequence list and cloned into LentiCRISPRv2 (Addgene plasmid #52961, a gift from Feng Zhang) (Table Si). HAP1 MTHFD1^{KO} cells were transiently transfected with LentiCRISPRv2 containing the gRNAs by using TurboFectin (OriGene) according to manufacturer's instructions. Transfected cells were selected with puromycin (Thermo Fisher Scientific) for 48 hours. Resistant cells were then split into single cell clones for single clone picking. Knockout clones were verified by both Sanger sequencing and western blot.

For the generation of NUDT5 knockouts, gRNA target sequences were chosen from the Brunello library sequence list and cloned into a modified pX330 vector (Addgene plasmid #64324, a gift from Ralf Kuehn). K56R cells were transiently transfected using TurboFectin (Origene) according to manufacturer's instructions. 48 hours after transfection, mCherry positive cells were sorted out and expanded for another 72 hours prior to validation experiments.

The catalytically inactive NUDT5 mutant (E112Q) plasmid was generated with the Q5 Site-directed Mutagenesis Kit (NEB, E0554S) (Wright et al 2016). Virus was generated as previously described, transduced into MTHFD1^{K56R}, NUDT5^{KO} cells, and single clones generated.

### Bioenergetic Measurements (Extracellular Flux Analysis)

Cell lines were seeded in 96-well plates at 100,000 cells/well on the same day of the experiment. Before measurement, medium was exchanged for XF Base Medium (Agilent 102353-100) containing glucose (10 mM), sodium pyruvate (1 mM), L-glutamine (2 mM) and adjusted to pH 7.4. Cells were incubated for 1 h at 37° C. before measurement. Measurements were carried out on a Seahorse XF96 (Agilent) with a MitoStress test kit (Agilent, 103015-100), following the manufacturer's instructions. Oligomycin, FCCP, and a mix of Rotenone and Antimycin A were injected at desired timepoints at a final concentration of 1 M, 1.5 M and 0.6 M, respectively. Data were analyzed with SeahorseWave (Agilent) and Prism (Graph Pad). ATP production was calculated using the Bioenergetics spreadsheet from the Russell Jones lab.

### Labelled Analogue Chemical Synthesis

Isotope labelled compound was synthesized from commercially available 8-¹³C-adenine as described in Juen et al. 2016 (Juen et al., 2016). Solvents and inorganic reagents were obtained from Lactan, Carl Roth, Sigma-Aldrich or TCI and were used as received. Reagents were obtained from abcr, Enamine or Sigma-Aldrich and were used as received. 8-¹³C-adenine was obtained from Cambridge Isotope Laboratories. Automated preparative flash chromatography was performed using KP-SIL Biotage cartridges on a Biotage Isolera Prime. ¹H NMR and ¹³C NMR spectra were recorded on a Bruker Avance III 400 MHz NMR spectrometer. Chemical shifts are given in parts per million (ppm), coupling constants J are given in Hertz, and spin multiplicities are given as s (singlet), d (doublet), t (triplet), q (quartet), m (multiplet), or b (broad signal). HRMS was recorded on a maXis UHR-TOF (Bruker) with ESI.

RNA Extraction and Real-Time Polymerase Chain Reaction (qPCR)

Primers used for qPCR are listed in Table S1. After treatment of cell lines in the different media conditions, cells were collected and RNA was extract with the RNeasy Mini Kit (Qiagen). RNA was reverse-transcribed using the LunaScript RT SuperMix Kit (NEB) as per manufacterer's instructions. Quantitative PCR was performed with the Luna Universal qPCR Master Mix (NEB) on a Lightcycle 480 (Roche). Fold Change was calculated by the ΔΔCₜ method and normalized to the TUBB gene.

### γH2AX Immunofluorescence Staining

Cells were pretreated in different media conditions for 24 hours and then fixed in 4% para-formaldehyde, and permeabilized with Triton-X. The fixed cells were then blocked with 5% BSA, diluted in PBS, for 1 hour and then incubated overnight with anti-γH₂AX antibody (9718T, 1:500, Cell Signalling Technology) at 4° C. The next day, the cells were washed and incubated with anti-rabbit Alex-Fluor 546 and Hoechst for 1 hour room temperature. The cells were washed again with PBS prior to imaging. Foci were quantified using a custom CellProfiler pipeline.

### Cell Cycle Analysis

Cells were pretreated in different media conditions for 24 hours, trypsinized and washed 2 times with ice-cold PBS. After washing, cells were resuspended in 0.5 ml of ice-cold PBS. 2 ml of ice-cold 70% EtOH was then added to the cells, drop by drop, while gently vortexing. Cells were then stored at −20° C. for at least 20 minutes. After fixation, cells were resuspended in PBS for 5 minutes and then stained with propidium iodide (#4087S, Cell Signaling Technology) according to manufacturer's protocol.

### Targeted Metabolomics and Stable Isotope Tracing

All cell lines were incubated in the respective media conditions (full FBS media, dialyzed FBS media, and dialyzed FBS media supplemented with 1 mM isotope labelled formate or 1 mM isotope labelled formate and 50 μM isotope labelled adenosine) for 24 hours prior to collection (Formate, Cambridge Isotope Laboratories, CDLM-6203-0.5)(Adenosine, Silantes, 125303601). Cells were collected and counted and 5 million cells for each replicate were washed with 1×PBS, pelleted, immediately snap-frozen, and stored at −80° C.

Cell extraction was performed in 1.5 mL Eppendorf tubes by adding 500 μL of ice-cold 80:20 (v/v) MeOH:H₂O solution to the cell pellet and vigorously vortexing. Samples were centrifuged at 10,000 g for 10 min at 4° C. before transferring the cell extract supernatant into 1.5 mL HPLC vials. The extraction of the cell pellets was repeated a second time and supernatants of the same samples were combined.

Cell extract samples were dried using a nitrogen evaporator. The dried residue was reconstituted in 50 μL water. An aliquot of 10 μL reconstituted sample extract was mixed with 10 μL of isotopically labelled internal standard mixture in a HPLC vial, vortexed and used for the metabolite analysis.

A 1290 Infinity II UHPLC system (Agilent Technologies) coupled with a 6470 triple quadrupole mass spectrometer (Agilent Technologies) was used for the LC-MS/MS analysis. The chromatographic separation for samples was carried out on a ZORBAX RRHD Extend-C18, 2.1×150 mm, 1.8 μm analytical column (Agilent Technologies). The column was maintained at a temperature of 40° C. and 4 μL of sample was injected per run. The mobile phase A was 3% methanol (v/v), 10 mM tributylamine, 15 mM acetic acid in water and mobile phase B was 10 mM tributylamine, 15 mM acetic acid in methanol. The gradient elution with a flow rate of 0.25 mL/min was performed for a total time of 24 min. Afterwards a back flushing of the column using a 6-port/2-position divert valve was carried out for 8 min using acetonitrile, followed by 8 min of column equilibration with 100% mobile phase A.

The triple quadrupole mass spectrometer was operated in an electrospray ionization negative mode, spray voltage 2 kV, gas temperature 150° C., gas flow 1.3 L/min, nebulizer 45 psi, sheath gas temperature 325° C., sheath gas flow 12 L/min. The metabolites of interest were detected using a dynamic MRM mode. The MassHunter 10.0 software (Agilent Technologies) was used for the data processing. Ten-point linear calibration curves with internal standardization were constructed for the quantification of metabolites.

### Intron Tagging Plasmid Cloning

The intron tagging plasmids were cloned as follows. The generic sgRNA targeting

plasmid and the GFP-donor or mCherry-donor plasmid were generated as previously described (Reicher et al., 2020). Briefly, the pX330 plasmid expression Cas9 and the generic sgRNA targeting the donor plasmid was generated by digesting pU6-(BbsI)_CBh-Cas9-T2A-mCherry (Addgene #64324) with BbsI followed by ligation with an annealed oligo duplex. mCherry was replaced with a Blasticidin resistance (BSD) using Gibson Assembly.

The GFP-donor plasmid containing the coding sequence of EGFP flanked by generic sgRNA targeting sites, splice acceptor and slice donor sites and 20 amino acid linkers was assembled from 4 fragments using Gibson assembly. The DNA fragment with a 25 nucleotide overlap to the pUC19 vector and 32 nucleotide overlap to the N-terminus of EGFP was generated from overlapping oligos (Sigma) and is comprised of a generic sgRNA targeting site that is not present in the human genome followed by a splice acceptor site and a flexible 20 amino acid glycine-serine linker. This fragment is followed by a fragment with the coding sequence of EGFP without a start or stop codon that was generated by PCR. The third fragment has a 27 nucleotide overlap to the C-terminus of EGFP and a 25 nucleotide overlap to the pUC19 vector and was generated from overlapping oligos (Sigma) and comprises a flexible 20 amino acid glycine-serine linker followed by a splice donor site and the generic sgRNA targeting site. The pUC19 vector was linearized by PCR for Gibson Assembly (NEBuilder HiFi DNA Assembly) with the other three fragments. The mCherry-donor plasmid was similarly constructed.

The plasmid containing the RPA2 intron sgRNA sequence was designed on Benchling and cloned as previously described. The sequence is listed in table S1.

### Generation of Intron Tagged Cell Lines

The three plasmids (intron targeting, donor targeting, GFP/mCherry donor) were transfected into WT HAP1, MTHFD1^{KO}, or K56R cells using Turbofectin (OriGene) as per the manufacturer's instructions. After 48 hours, cells were collected and sorted by flow cytometry using a Sony-Cell Sorter SH800ZD. These cells were then diluted and split into single clones. After expansion, the colonies were visualized on an Opera Phenix (Perkin Elmer) to determine clones successfully tagged with the GFP/mCherry at the intron.

### Synthesis and Compound Characterization

Commercial reagents and solvents were purchased from commercial suppliers and used without further purification. All reactions involving moisture sensitive reagents were carried out under a nitrogen atmosphere using standard vacuum line techniques and dry solvents.

An Elga DV 25 system was used for deionizing water.

Thin layer chromatography was performed on aluminium plates coated with 60 F254 silica gel. Plates were visualized using UV light (254 nm).

Flash column chromatography was performed on a Biotage Isolera one flash column chromatography platform.

¹H and ¹³C NMR spectra were obtained using Bruker NMR spectrometers (400 MHz). The proton and carbon chemical shift values are reported in parts per million (ppm, δ scale) downfield from tetramethylsilane (TMS) and the indicated solvent. NMR spectra were processed and analyzed using MestReNova software. Spin multiplicities are given as s (singlet), d (doublet), t (triplet), q (quartet), dd (doublet of doublets), m (multiplet) and b (broad), coupling constants J are given in hertz (Hz), and signal area integration in natural numbers.

LCMS was performed with a Kinetex 5μ EVO C18 100A 100×3.0 mm column on a Waters SFO and 515 HPLC pump and Waters Binary Gradient 2545 device using linear gradient of solvent A (93% H2O, 5% acetonitrile and 2% of 0.5 M ammonium acetate pH 6.0) and solvent B (18% H2O, 80% acetonitrile and 2% ammonium acetate pH 6.0), eluting at a flow rate of 2 mL/min: 5% B for 0.35 min, 5% B to 95% B for 1 min, 95% to 5% B for 0.1 min and 5% B for 0.8 min. LCMS was used as a measure of compound purity using either UV absorbance (Waters UV/visible Detector 2489), ELSD signal (Waters ELS Detector 2424) or ESI+TIC (SQ Detector 2). Preparative HPLC was performed on the same system with a Kinetex 5u EVO C18 100A 150×21.2 mm column using linear gradient of solvent A over 20 min from 85% to 10% eluting at a flow rate of 20 mL/min. LCMS was acquired using Waters FractionLynx software and processed using MestReNova software.

### Purification, Crystallization and Structure Determination

NUDT5 (residues 1-208) was cloned into a pNIC28-Bsa4 bacterial expression vector. Protein expression was performed in Rosetta™ (DE3) cells and purification was performed as previously described [3]. NUDT5 at 20 mg/ml concentration was complexed with PROTAC and co-crystalized in a buffer 0.1M Tris, pH 8.5, 0.2 M Sodium Acetate, 30% PEG 4000 at 20° C. The diffraction datasets were collected at Diamond Light Source beamline I03. Data integration and scaling were performed using XDS [4] and AIMLESS [5], respectively. Phases were obtained using PDB ID: 5NWH as the search model in Molecular Replacement in PHASER [6]. Final structure was obtained after multiple rounds manual building in COOT [7] and refining with Refmac5 [8]. Structural validation was performed using MolProbity [9] and structures were visualized with PyMOL. Crystallographic data statistics are summarized in Table S2.

### Immunoprecipitation

HAP-1 WT cells were treated with 100 nM TH5427, dNUDT5 or DMSO for 20 h in a 100 mm dish. Cells were washed four times with 2 mL PBS before lysing with 500 μL 0.8% NP-40 based lysis buffer (Tris [pH 7.5], 0.8% NP-40, 5% glycerol, 1.5 mM MgCl2, 100 mM NaCl, 25 mM NaF, 1 mM Na3VO4, 1 mM PMSF, 1 mM DTT, 10 μg/mL TPCK, 1 μg/mL Leupeptin, 1 μg/mL Aprotinin, 10 μg/mL soybean trypsin inhibitor). The cell suspension was transferred to a 2 mL reaction tube, incubated on ice for 30 min and then centrifuged for 30 min at 20000×g (4° C.). Cleared lysates were spiked with 1 μg of PPAT (15401-1-AP, Proteintech) or rabbit IgG control antibody (3900S, Cell Signaling) and incubated on a wheel at 4° C. overnight. 30 μL magnetic protein G beads slurry (#1003D, ThermoFisher) was incubated with lysate/antibody mix for 15 min on room temperature on a wheel. After 3× washing with 1 mL lysis buffer protein were eluted with 30 μL 2× Laemmli buffer by boiling for 10 min at 70° C.

### Chemical Pulldown

Profiling of the TH5427 proteome wide specificity was performed in triplicates as previously described [10](Huber et. al. 2014 Nature). In brief, NHS-activated sepharose beads (17090601, Cytiva) were derivatised with a amine functionalised TH5427 analog (c-TH5427) (manuscript in preparation) at a coupling density of 0.5 mM with 0.75 μL TEA per 50 μL beads in DMSO. Free binding sites were blocked by 2.5 μL ethanolamine per 50 μL beads. Beads were washed with excess of DMSO and 0.8% NP-40 based lysis buffer. HAP-1 WT and HAP-1 MTHFD1 K56R cell pellets were thawed on ice and lysed with 3× excess lysis buffer containing 1 μL/mL benzonase. The cell suspension was drawn through a 21G needle 10× before clearing cell debris by centrifugation for 30 min at 20000×g (4° C.) in a table-top centrifuge. 20 μM of TH5427 or DMSO was spiked into 1 mL of lysate (5 mg/mL and incubated for 30 min on a wheel at 4° C. After mixing cell lysates with 50 uL beads for 2 h on a wheel at 4° C. the suspension was transferred in filter columns, washed 4× 1 mL lysis buffer and subsequently eluted with 2× 50 μL 2× Laemmli buffer by heating for 5 min at 95° C. Elution fractions were used for gel loading or digested for LC-MS/MS analysis.

### Proteomics

### Untargeted Global Proteomics and Chemoproteomics

Proteomics samples were digested with trypsin using STrap columns following the manufacturer's protocol (C02-micro, ProtiFi). For untargeted global proteomics HAP1 cells were treated with DMSO or 100 nM dNUDT5 for 6 h or 24 h. Then, the cell pellets were collected and lysed in 5% SDS containing 1 μL/mL benzonase (E1014, Millipore) and subsequent short sonication to shear genomic DNA (1× 2 s pulse, 20% amplitude, microtip, Sonics Vibra Cell). 50 μg protein from the total cell lysate or 80% of the elution fraction of the chemical pulldown were first reduced by 20 mM dithiothreitol (M02712, Fluorochem), and alkylated by 40 mM iodoacetamide (I1149, Sigma). Phosphoric acid and S-Trap protein binding buffer were added into the sample lysates. Then, the SDS lysate/S-Trap buffer were loaded into a S-Trap column (C02-micro-80, ProtiFi). 1 μg of trypsin (V5111, Promega) was used to digest each sample at 37° C. for 20 h. The samples were then eluted and dried by vacuum centrifugation. Peptide pellets were resuspended in mass spectrometry grade water with 2% acetonitrile (85188, Thermo Scientific) and 0.1% trifluoroacetic acid (85183, Thermo Scientific). The samples were injected into Orbitrap Fusion™ Lumos™ Tribrid™ Mass Spectrometer (Thermo Scientific) or QE for untargeted global protemics or chemoproteomics, respectively.

### Data Analysis

Raw data were searched against the human database (UP000005640, downloaded 08/21) using DIA-NN (Demichev et al. 2020 Nature Methods) by enabling FASTA digest for library free search and Deep learning-based spectra, RTs and IMs prediction. Trypsin with 1 missed cleavage and Ox(M) and Ac(N-term) were enabled. Precursor FDR was kept at 1% and MBR enabled. The protein groups matrix was further analysed using Perseus (1.6.2.1) or R studio (4.1.1). Potential contaminants in the chemoproteomic data were filtered against the top 300 most common Homo sapiens contaminants reported in CRAPome (Mellacheruvu et al. 2013 Nature Methods). Proteins were removed if detected less than 2 times in each group. After column-wise normalisation by median subtraction volcano plots were generated by calculating fold change and −log 10(p-value) from a both-sided t-test.

### NanoBRET Assay

For NanoBRET target engagement assay, intracellular TE Nano-Glo substrate/inhibitor (N2161, Promega) was used according to the manufacturer's instruction. In brief, 200,000 HEK293T cells/ml was seeded in a T-75 flask. N-terminal NanoLuciferse NUDT5 was reverse transfected into the cells using Fugene HD transfection reagent (E2312, Promega). After 24 h the transfected HEK293 (200,000 cells/ml) were replated onto a 384-well plate containing compounds. 2.5 nM NU008116a was used as the final tracer concentration. After 2 h, NanoLuc substrate and inhibitor was added into each well and the signals were measured by PHERAstar Microplate Reader.

For NanoBRET protein-protein interaction assay, NanoBRET Nano-Glo Detection System (N1662, Promega) was used according to the manufacturer's instruction. In brief, 220,000 HEK293 cells/ml was seeded in a 6-well plate. After 6 h, N-terminal nanoLuc NUDT5 and Halo-tag CRBN were then transfected into the cells using Fugene HD transfection reagent (E2312, Promega). The transfected HEK293 (220,000 cells/ml) were then replated onto a 384-well plate containing compounds. After 4 h incubation, NanoLuc substrate was added into each well and the signals were measured by PHERAstar Microplate Reader.

Compounds were dispensed in the 384-well plates by using Echo Acoustic Dispenser.

### 6-Thioguanine Assay

NB-4 and HAP1 cells (1000 cells/well in 40 uL) were seeded onto a 384-well plate containing compounds. DMSO or 0.2 μg/ml 6-thioguanine (A4882, Sigma-Aldrich) was added into respective wells. After 72 h incubation cell viability was measured by using CellTiter-Glo 2.0 Assay (G9242, Promega). Compounds in the 384-well plates were prepared by using Echo Acoustic Dispenser.

### Statistical Analysis

All statistical tests were performed in Prism (Prism 8). For comparisons among three or more conditions, ANOVA was performed with Tukey's HSD to correct for multiple comparisons. For comparisons between two conditions, T-tests were performed with a two-tailed p-value.

### Tables

TABLE S1
Table of primer sequences used for CRISPR KO,
qPCR and intron tagging

| qPCR Primer | Sequence |
|---|---|
| TUBB FWD | TACTACAATGAAGCCACA |
| TUBB REV | AGACTGACCAAATACAAAG |
| PKM1 FWD | TCACTCCACAGACCTCATGG |
| PKM1 REV | GAAGATGCCACGGTACAGGT |
| PKM2 FWD | ATCGTCCTCACCAAGTCTGG |
| PKM2 REV | GAAGATGCCACGGTACAGGT |

sgRNA Target Sequence for CRISPR KO

| | |
|---|---|
| PML gRNA 1 | GCGGTACCAGCGCGACTACG |
| PML gRNA 2 | CTGCGCGTGAACCGCGCCAA |
| PARP8 gRNA 1 | TTTCAAGGAACCTAACGCAG |
| PARP8 gRNA 2 | TTTGTGTTGTGCTCACATCG |
| ATF7 guide 1 | AGCCCACCCCTAGTACTGGG |
| ATF7 guide 2 | CCCAACCTCTGTCATCACAC |
| NUDT5 gRNA1 | ACAACGTACATGGATCCTAC |
| NUDT5 gRNA2 | CAAACAGTATATCATTTCAG |

sgRNA Target Sequence for RPA2 intron tag

| | |
|---|---|
| RPA2 Intron | GTCCCTGCCATCAAGCAGGG |

TABLE S2
Data collection and refinement statistics for dNUDT5 bound to NUDT5

| Resolution range (Å) | 56.72-2.001 | (2.073-2.001) |
|---|---|---|
| Space group | C 1 2 1 | |
| Unit cell a, b, c (α, β, γ) | 113.55 40.202 99.945 90 121.922 90 | |
| Total reflections | 179823 | (17066) |
| Unique reflections | 26218 | (2577) |
| Multiplicity | 6.9 | (6.6) |
| Completeness (%) | 99.74 | (98.69) |
| Mean I/sigma(I) | 8.69 | (0.51) |
| Wilson B-factor | 39.43 | |
| R-merge | 0.1231 | (1.417) |
| R-meas | 0.1333 | (1.538) |
| R-pim | 0.05052 | (0.5925) |
| CC1/2 | 0.997 | (0.585) |
| CC* | 0.999 | (0.859) |
| Refinement | | |
| Reflections used in refinement | 26173 | (2566) |
| Reflections used for R-free | 1989 | (199) |
| R-work | 0.2025 | (0.3558) |
| R-free | 0.2485 | (0.3986) |

| | |
|---|---|
| Number of non-hydrogen atoms | 3194 |
| macromolecules | 2980 |
| ligands | 76 |
| solvent | 138 |
| Protein residues | 388 |
| RMS(bonds) | 0.008 |
| RMS(angles) | 0.96 |
| Ramachandran favored (%) | 96.60 |
| Ramachandran allowed (%) | 3.14 |
| Ramachandran outliers (%) | 0.26 |
| Rotamer outliers (%) | 1.24 |
| Clashscore | 4.02 |
| Average B-factor | 47.09 |
| macromolecules | 46.47 |
| ligands | 72.85 |
| solvent | 46.15 |

Statistics for the highest-resolution shell are shown in parentheses.

### REFERENCES IN INTRODUCTION AND EXAMPLES

1. G. S. Ducker, J. D. Rabinowitz, One-Carbon Metabolism in Health and Disease. Cell Metab 25, 27-42 (2017).
2. P. Burda et al., Characterization and review of MTHFD1 deficiency: four new patients, cellular delineation and response to folic and folinic acid treatment. J Inherit Metab Dis 38, 863-872 (2015).
3. H. Fu et al., Folate Intake and Risk of Pancreatic Cancer: A Systematic Review and Updated Meta-Analysis of Epidemiological Studies. Dig Dis Sci 66, 2368-2379 (2021).
4. A. J. MacFarlane et al., Nuclear localization of de novo thymidylate biosynthesis pathway is required to prevent uracil accumulation in DNA. J Biol Chem 286, 44015-44022 (2011).
5. R. Pieroth, S. Paver, S. Day, C. Lammersfeld, Folate and Its Impact on Cancer Risk. Curr Nutr Rep 7, 70-84 (2018).
6. P. J. Stover, One-carbon metabolism-genome interactions in folate-associated pathologies. J Nutr 139, 2402-2405 (2009).
7. N. Gonen, Y. G. Assaraf, Antifolates in cancer therapy: structure, activity and mechanisms of drug resistance. Drug Resist Updat 15, 183-210 (2012).
8. G. Peters, J. H. Hooijberg, G. Kaspers, G. Jansen, Folates and antifolates in the treatment of cancer; role of folic acid supplementation on efficacy of folate and non-folate drugs. Trends in Food Science & Technology 16, 289-297 (2005).
9. M. S. Field, E. Kamynina, J. Chon, P. J. Stover, Nuclear Folate Metabolism. Annu Rev Nutr 38, 219-243 (2018).
10. S. Sdelci et al., MTHFD1 interaction with BRD4 links folate metabolism to transcriptional regulation. Nat Genet 51, 990-998 (2019).
11. G. S. Ducker et al., Reversal of Cytosolic One-Carbon Flux Compensates for Loss of the Mitochondrial Folate Pathway. Cell Metab 23, 1140-1153 (2016).
12. G. Burgos-Barragan et al., Mammals divert endogenous genotoxic formaldehyde into one-carbon metabolism. Nature 548, 549-554 (2017).
13. Y. Zheng et al., Mitochondrial One-Carbon Pathway Supports Cytosolic Folate Integrity in Cancer Cells. Cell 175, 1546-1560 e1517 (2018).
14. J. He, L. N. Zou, V. Pareek, S. J. Benkovic, Multienzyme interactions of the de novo purine biosynthetic protein PAICS facilitate purinosome formation and metabolic channeling. J Biol Chem 298, 101853 (2022).
15. C. Staben, J. C. Rabinowitz, Nucleotide sequence of the Saccharomyces cerevisiae ADE3 gene encoding C1-tetrahydrofolate synthase. J Biol Chem 261, 4629-4637 (1986).
16. J. M. Song, J. C. Rabinowitz, Function of yeast cytoplasmic C₁-tetrahydrofolate synthase. Proc Natl Acad Sci USA 90, 2636-2640 (1993).
17. C. K. Barlowe, D. R. Appling, Molecular genetic analysis of Saccharomyces cerevisiae C1-tetrahydrofolate synthase mutants reveals a noncatalytic function of the ADE3 gene product and an additional folate-dependent enzyme. Mol Cell Biol 10, 5679-5687 (1990).
18. C. K. Barlowe, M. E. Williams, J. C. Rabinowitz, D. R. Appling, Site-directed mutagenesis of yeast C1-tetrahydrofolate synthase: analysis of an overlapping active site in a multifunctional enzyme. Biochemistry 28, 2099-2106 (1989).
19. M. G. West, D. W. Horne, D. R. Appling, Metabolic role of cytoplasmic isozymes of 5,10-methylenetetrahydrofolate dehydrogenase in Saccharomyces cerevisiae. Biochemistry 35, 3122-3132 (1996).
20. K. E. Christensen, H. Patel, U. Kuzmanov, N. R. Mejia, R. E. MacKenzie, Disruption of the mthfd1 gene reveals a monofunctional 10-formyltetrahydrofolate synthetase in mammalian mitochondria. J Biol Chem 280, 7597-7602 (2005).
21. M. S. Field, E. Kamynina, D. Watkins, D. S. Rosenblatt, P. J. Stover, Human mutations in methylenetetrahydrofolate dehydrogenase 1 impair nuclear de novo thymidylate biosynthesis. Proc Natl Acad Sci USA 112, 400-405 (2015).
22. V. A. Blomen et al., Gene essentiality and synthetic lethality in haploid human cells. Science 350, 1092-1096 (2015).
23. R. J. Torres, J. G. Puig, Hypoxanthine-guanine phosophoribosyltransferase (HPRT) deficiency: Lesch-Nyhan syndrome. Orphanet J Rare Dis 2, 48 (2007).
24. M. Vogel et al., HPRT and Purine Salvaging Are Critical for Hematopoietic Stem Cell Function. Stem Cells 37, 1606-1614 (2019).
25. R. H. Wright et al., ADP-ribose-derived nuclear ATP synthesis by NUDIX5 is required for chromatin remodeling. Science 352, 1221-1225 (2016).
26. M. Zha, C. Zhong, Y. Peng, H. Hu, J. Ding, Crystal structures of human NUDT5 reveal insights into the structural basis of the substrate specificity. J Mol Biol 364, 1021-1033 (2006).
27. B. D. G. Page et al., Targeted NUDT5 inhibitors block hormone signaling in breast cancer cells. Nat Commun 9, 250 (2018).
28. U. Rix, G. Superti-Furga, Target profiling of small molecules by chemical proteomics. Nat Chem Biol 5, 616-624 (2009).
29. E. L. Huttlin et al., Dual proteome-scale networks reveal cell-specific remodeling of the human interactome. Cell 184, 3022-3040 e3028 (2021).
30. J. G. Doench et al., Optimized sgRNA design to maximize activity and minimize off-target effects of CRISPR-Cas9. Nat Biotechnol 34, 184-191 (2016).
31. E. W. Holmes, J. B. Wyngaarden, W. N. Kelley, Human glutamine phosphoribosylpyrophosphate amidotransferase. Two molecular forms interconvertible by purine ribonucleotides and phosphoribosylpyrophosphate. J Biol Chem 248, 6035-6040 (1973).
32. E. Zoref-Shani, O. Sperling, Dependence of the metabolic fate of IMP on the rate of total IMP synthesis. Studies in cultured fibroblasts from normal subjects and from purine-overproducing mutant patients. Biochim Biophys Acta 607, 503-511 (1980).

### ASPECTS OF THE INVENTION

1. A compound of formula (I):

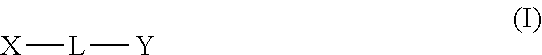

or a pharmaceutically acceptable salt thereof,
wherein X is a moiety capable of binding to NUDT5;
L is a linker comprising at least one heterocyclic moiety, said heterocyclic moiety being a bivalent, saturated, nitrogen-containing heterocyclic moiety; and
Y is a moiety capable of binding to E3 ligase.
2. The compound or pharmaceutically acceptable salt according to aspect 1 wherein the heterocyclic moiety is a 5- to 7-membered heterocyclic moiety.
3. The compound or pharmaceutically acceptable salt according to aspect 1 or aspect 2 wherein the heterocyclic moiety comprises one or two nitrogen atoms.
4. The compound or pharmaceutically acceptable salt according to any preceding aspect wherein the heterocyclic moiety is selected from the following:

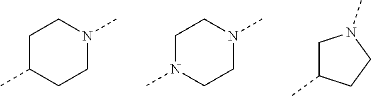

wherein a dotted line indicates a covalent bond attaching the heterocyclic moiety to X, Y, or another portion of linker L.
5. The compound or pharmaceutically acceptable salt according to any preceding aspect wherein linker L comprises a bicyclic moiety of formula (II):

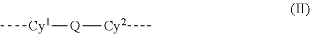

wherein:
Cy¹ and Cy² are each bivalent, saturated, nitrogen-containing heterocyclic moieties;
Q is selected from a covalent bond, C₁-C₆ alkylene; —NH—, —O—, —C(═O)—, and any combination thereof; and
a dotted line indicates a covalent bond attaching the bicyclic moiety of formula (II) to X, Y, or another portion of linker L.
6. The compound or pharmaceutically acceptable salt according to aspect 5 wherein the linker L comprises a bicyclic moiety of formula (III):

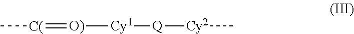

wherein Cy¹, Cy², Q and the dotted lines are as defined in aspect 5.
7. The compound or pharmaceutically acceptable salt according to aspect 5 or aspect 6 wherein Cy¹ and Cy² are each independently as defined in any one of claims 2 to 4.
8. The compound according to any preceding aspect wherein L is:

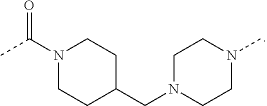

9. The compound according to any preceding aspect wherein L is:

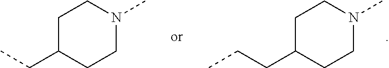

10. A compound of formula (IA):

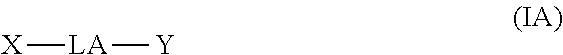

or a pharmaceutically acceptable salt thereof,
wherein X is a moiety capable of binding to NUDT5;
LA is a linker of formula:

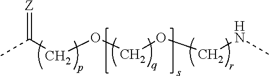

wherein
Z is O or S;
p, q, r and s are each integers independently selected from 1, 2, 3 or 4, each q being independently selected from 1, 2, 3 or 4 where s>1; and
Y is a moiety capable of binding to E3 ligase.
11. The compound or pharmaceutically acceptable salt according to aspect 10 wherein p, q and r are all 2.
12. The compound or pharmaceutically acceptable salt according to any preceding aspect wherein L or LA is substituted by 0, 1 or 2 substituents each independently selected from halo, C₁-C₆ alkyl, or C₁-C₆ alkoxy; preferably wherein L or LA is substituted by 0 substituents.
13. The compound or pharmaceutically acceptable salt according to any preceding aspect wherein X is selected from:

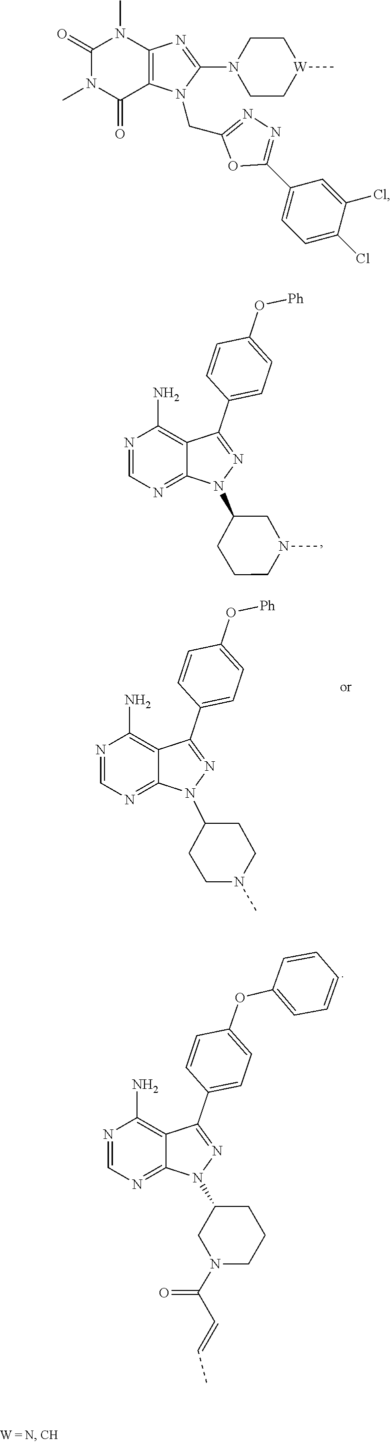

14. The compound or pharmaceutically acceptable salt according to any preceding aspect wherein Y is selected from:

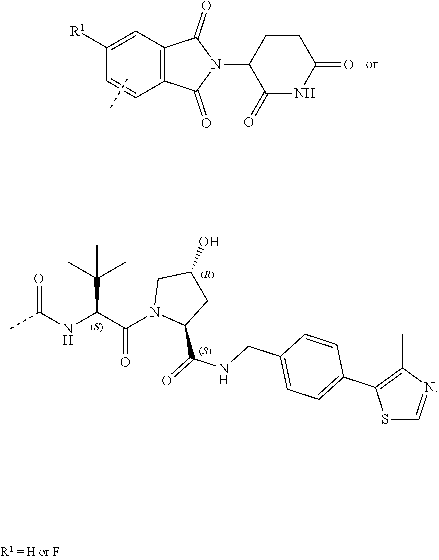

15. The compound or pharmaceutically acceptable salt according to any preceding aspect wherein X is

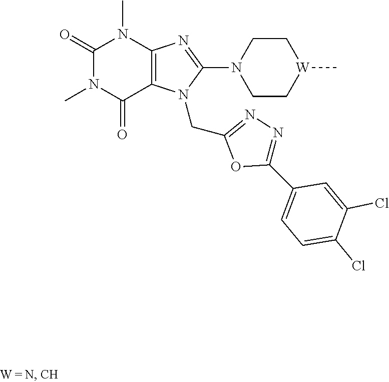

### and Y is

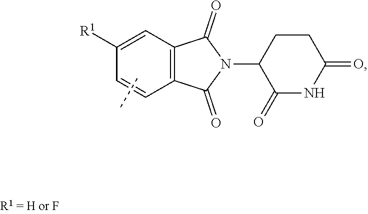

preferably wherein W is N and R¹ is H.
16. The compound or pharmaceutically acceptable salt according to any preceding aspect which is selected from

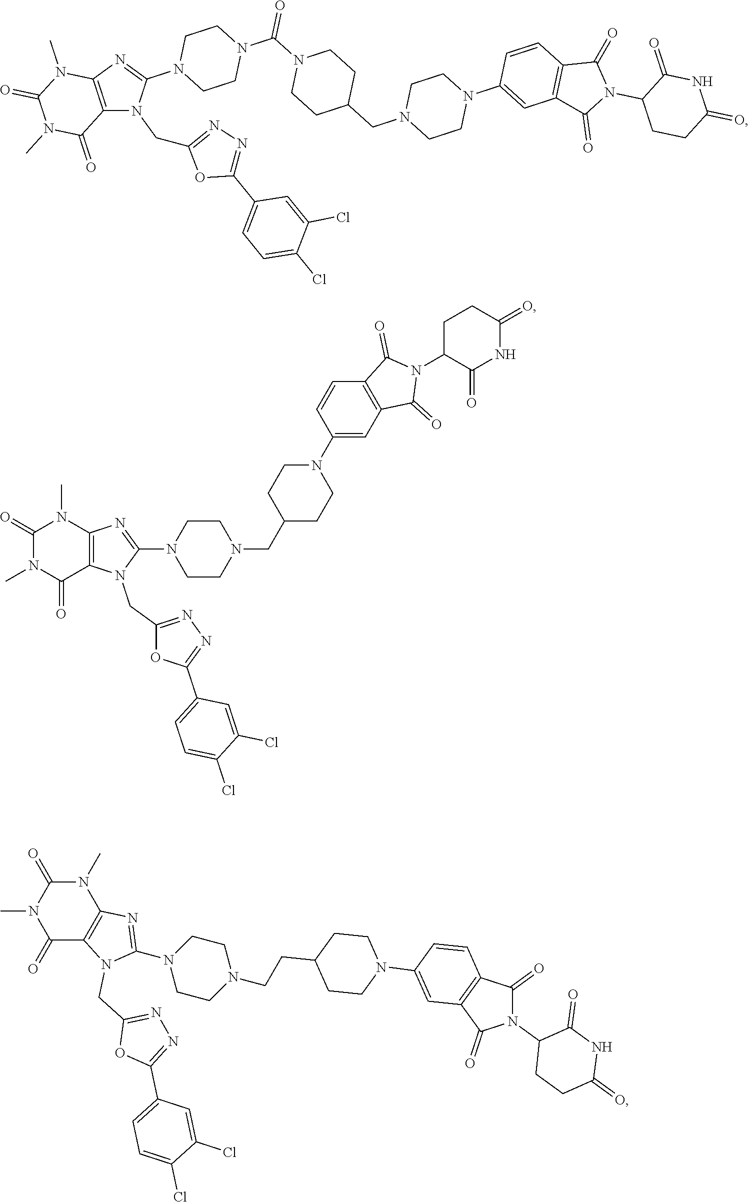 [SMILES: Cn1c(=O)c2c(nc(N3CCN(C(=O)N4CCC(CN5CCN(c6ccc7c(c6)C(=O)N(C6CCC(=O)NC6=O)C7=O)CC5)CC4)CC3)n2Cc2nnc(-c3ccc(Cl)c(Cl)c3)o2)n(C)c1=O ; Cn1c(=O)c2c(nc(N3CCN(CC4CCN(c5ccc6c(c5)C(=O)N(C5CCC(=O)NC5=O)C6=O)CC4)CC3)n2Cc2nnc(-c3ccc(Cl)c(Cl)c3)o2)n(C)c1=O ; Cn1c(=O)c2c(nc(N3CCN(CCC4CCN(c5ccc6c(c5)C(=O)N(C5CCC(=O)NC5=O)C6=O)CC4)CC3)n2Cc2nnc(-c3ccc(Cl)c(Cl)c3)o2)n(C)c1=O]

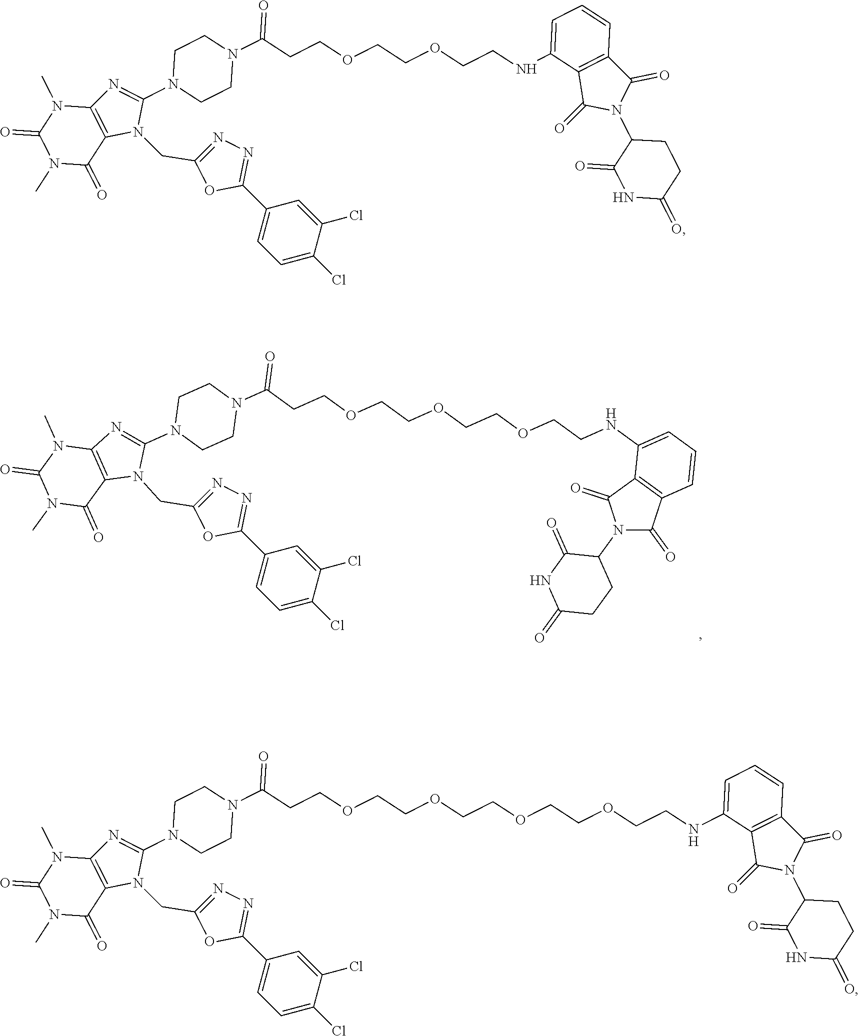 [SMILES: Cn1c(=O)c2c(nc(N3CCN(C(=O)CCOCCOCCNc4cccc5c4C(=O)N(C4CCC(=O)NC4=O)C5=O)CC3)n2Cc2nnc(-c3ccc(Cl)c(Cl)c3)o2)n(C)c1=O ; Cn1c(=O)c2c(nc(N3CCN(C(=O)CCOCCOCCOCCNc4cccc5c4C(=O)N(C4CCC(=O)NC4=O)C5=O)CC3)n2Cc2nnc(-c3ccc(Cl)c(Cl)c3)o2)n(C)c1=O ; Cn1c(=O)c2c(nc(N3CCN(C(=O)CCOCCOCCOCCOCCNc4cccc5c4C(=O)N(C4CCC(=O)NC4=O)C5=O)CC3)n2Cc2nnc(-c3ccc(Cl)c(Cl)c3)o2)n(C)c1=O]

or a pharmaceutically acceptable salt thereof.
17. The compound according to any preceding aspect which is:

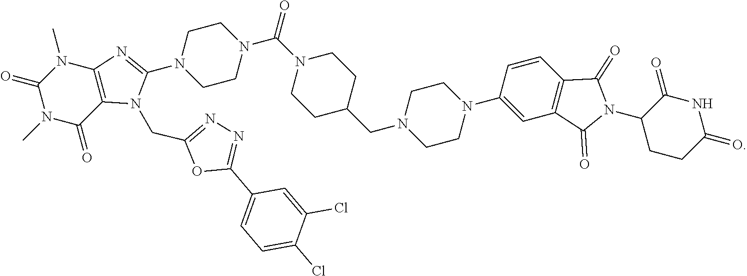 [SMILES: Cn1c(=O)c2c(nc(N3CCN(C(=O)N4CCC(CN5CCN(c6ccc7c(c6)C(=O)N(C6CCC(=O)NC6=O)C7=O)CC5)CC4)CC3)n2Cc2nnc(-c3ccc(Cl)c(Cl)c3)o2)n(C)c1=O]

18. A compound of formula (IB):

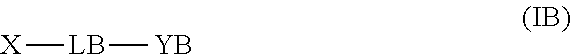

or a pharmaceutically acceptable salt thereof,
wherein X is a moiety capable of binding to NUDT5;
LB is a linker; and
YB is a moiety capable of recruiting cellular destruction machinery, except that YB is not a moiety capable of binding to VHL or CRBN.
19. The compound or pharmaceutically acceptable salt according to aspect 18, wherein:
(a) linker LB is a linker of formula L or LA as described herein; and/or
(b) YB is selected from a hydrophobic tag, an autophagy-targeting moiety, a moiety capable of binding to E2 ligase, a moiety capable of binding to E3 ligase other than a moiety capable of binding to VHL or CRBN, and a proteasome recruiter; and/or
(c) X is as defined in aspect 13, preferably as defined in aspect 15.
20. A pharmaceutical composition comprising a compound or pharmaceutically acceptable salt according to any one of aspects 1 to 19 and a pharmaceutically excipient or carrier.
21. A compound or pharmaceutically acceptable salt of any one of aspects 1 to 19 for use in the treatment of the human or animal body.
22. A compound or pharmaceutically acceptable salt of any one of aspects 1 to 19 for use in a method of treating an inflammatory disorder, autoimmune disorder, cancer or metabolic disorder in a subject.
23. A compound or pharmaceutically acceptable salt of any one of aspects 1 to 19 for use in a method of reducing the side-effects or toxicity of a second drug, wherein the subject is also being treated with said second drug.
24. The compound or pharmaceutically acceptable salt for the use of aspect 23, wherein the second drug is an anti-cancer drug or is an anti-arthritis drug.
25. The compound or pharmaceutically acceptable salt for the use of aspect 23, wherein the second drug is methotrexate or 6-thioguanine (6-TG).
26. The compound or pharmaceutically acceptable salt for the use of any one of aspects 23 to 25, wherein the second drug is being administered to treat an inflammatory disorder, autoimmune disorder, cancer or metabolic disorder in a subject.
27. The compound or pharmaceutically acceptable salt for the use of aspect 22 or 26, wherein the inflammatory disorder is arthritis, inflammatory bowel disorder, psoriasis or Crohn's disorder.
28. The compound or pharmaceutically acceptable salt for the use of aspect 27, wherein the arthritis is rheumatoid arthritis.
29. The compound or pharmaceutically acceptable salt for the use of aspect 22 or 26, wherein the cancer is a leukaemia, breast cancer, head cancer, neck cancer, sarcoma, or a gestational choriocarcinoma.
30. A compound or pharmaceutically acceptable salt according to any one of aspects 1 to 19 and methotrexate for simultaneous, separate, or sequential administration to treat cancer or arthritis.
31. A compound or pharmaceutically acceptable salt according to any one of aspects 1 to 19 and 6-thioguanine for simultaneous, separate, or sequential administration to treat cancer.
32. The pharmaceutical composition of aspect 20, further comprising methotrexate.
33. The pharmaceutical composition of aspect 20, further comprising 6-thioguanine.
34. A compound or pharmaceutically acceptable salt according to any one of aspects 1 to 19 for use in a method of treatment of a subjecting by inhibiting NUDT14.
35. The compound or pharmaceutically acceptable salt for the use of aspect 34, wherein the compound inhibits both NUDT14 and NUDT5.
36. A compound able to reduce the association of NUDT5 and phosphoribosyl pyrophosphate amidotransferase (PPAT) for use in a method of treatment.
37. A compound able to reduce the association of NUDT5 and phosphoribosyl pyrophosphate amidotransferase (PPAT) for use in a method of treating an inflammatory disorder, autoimmune disorder, cancer or metabolic disorder in a subject.
38. The compound for use in the method of aspect 37, wherein the compound is given to a subject also treated with a second drug, with the compound reducing a side-effect or the toxicity of the second drug.
39. The compound for use in the method of any one of aspects 36 to 38, wherein the compound is as defined in any one of aspects 1 to 19.
40. A method of screening for a therapeutic agent comprising determining the ability of a test agent to reduce the association between NUDT5 and PPAT.
41. The method of aspect 40, wherein the method further comprises assessing the ability of test agents able to reduce the association between NUDT5 and PPAT to reduce the side-effects or toxicity of methotrexate or 6-thioguanine.
42. The method of aspect 40 or 41, wherein if the test agent is able to reduce the association between NUDT5 and PPAT formulating the agent with a pharmaceutic carrier or excipient to produce a pharmaceutical composition.

1. A compound of formula (I): 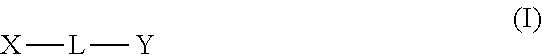
or a pharmaceutically acceptable salt thereof,
wherein X is a moiety capable of binding to NUDT5;
L is a linker comprising at least one heterocyclic moiety, said heterocyclic moiety being a bivalent, saturated, nitrogen-containing heterocyclic moiety; and
Y is a moiety capable of binding to E3 ligase.

2. The compound or pharmaceutically acceptable salt according to claim 1 wherein the heterocyclic moiety is a 5- to 7-membered heterocyclic moiety.

3. The compound or pharmaceutically acceptable salt according to claim 1 wherein the heterocyclic moiety comprises one or two nitrogen atoms.

4. The compound or pharmaceutically acceptable salt according to claim 1 wherein the heterocyclic moiety is selected from the following: 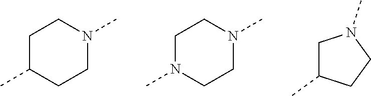
wherein a dotted line indicates a covalent bond attaching the heterocyclic moiety to X, Y, or another portion of linker L.

5. The compound or pharmaceutically acceptable salt according to claim 1 wherein linker L comprises a bicyclic moiety of formula (II): 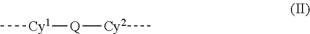
wherein:
Cy¹ and Cy² are each bivalent, saturated, nitrogen-containing heterocyclic moieties;
Q is selected from a covalent bond, C₁-C₆ alkylene; —NH—, —O—, —C(═O)—, and any combination thereof; and
a dotted line indicates a covalent bond attaching the bicyclic moiety of formula (II) to X, Y, or another portion of linker L.

6. The compound or pharmaceutically acceptable salt according to claim 5 wherein the linker L comprises a bicyclic moiety of formula (III): 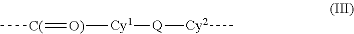
wherein Cy¹, Cy², Q and the dotted lines are as defined in claim 5.

7. The compound or pharmaceutically acceptable salt according to claim 5 wherein Cy¹ and Cy² are each independently claim a 5- to 7-membered heterocyclic moiety, optionally each comprising one or two nitrogen atoms, and further optionally each selected from the following: 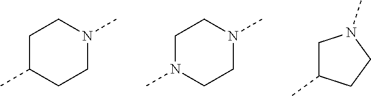
wherein a dotted line indicates a covalent bond attaching the heterocyclic moiety to X, Y, or another portion of linker L.

8. The compound according to claim 1 wherein L is:

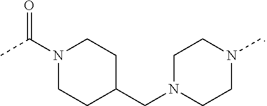

9. The compound according to claim 1 wherein L is:

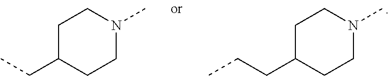

10. A compound of formula (IA): 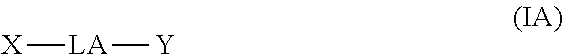
or a pharmaceutically acceptable salt thereof,
wherein X is a moiety capable of binding to NUDT5;
LA is a linker of formula: 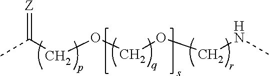
wherein
Z is O or S;
p, q, r and s are each integers independently selected from 1, 2, 3 or 4, each q being independently selected from 1, 2, 3 or 4 where s>1; and
Y is a moiety capable of binding to E3 ligase.

11. The compound or pharmaceutically acceptable salt according to claim 10 wherein p, q and r are all 2.

12. The compound or pharmaceutically acceptable salt according to claim 1 wherein L is substituted by 0, 1 or 2 substituents each independently selected from halo, C₁-C₆ alkyl, or C₁-C₆ alkoxy; optionally wherein L is substituted by 0 substituents.

13. The compound or pharmaceutically acceptable salt according to claim 1 wherein X is selected from:

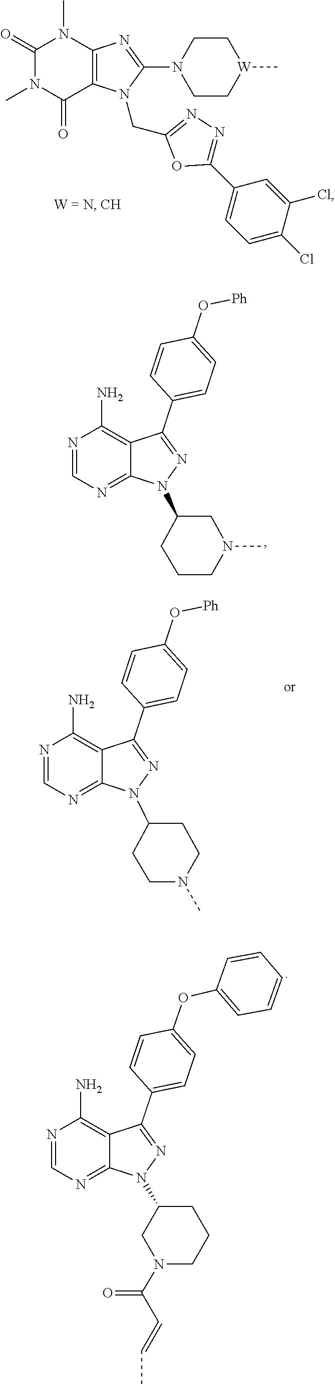

14. The compound or pharmaceutically acceptable salt according to claim 1 wherein Y is selected from:

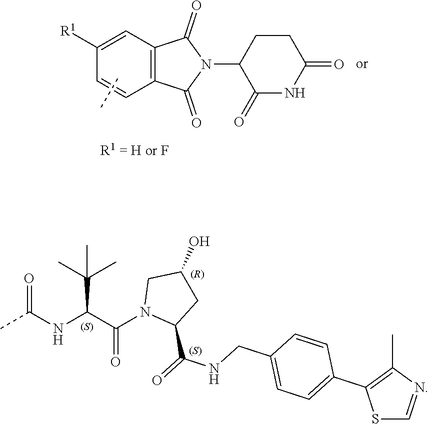

15. The compound or pharmaceutically acceptable salt according to claim 1 wherein X is 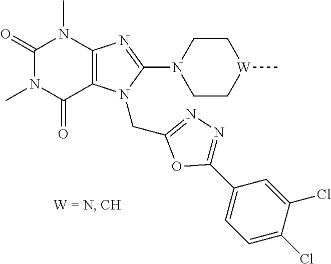
and Y is 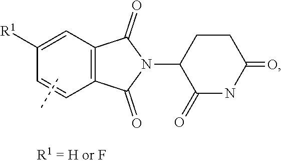
optionally wherein W is N and R¹ is H.

16. The compound or pharmaceutically acceptable salt according to claim 1 which is selected from 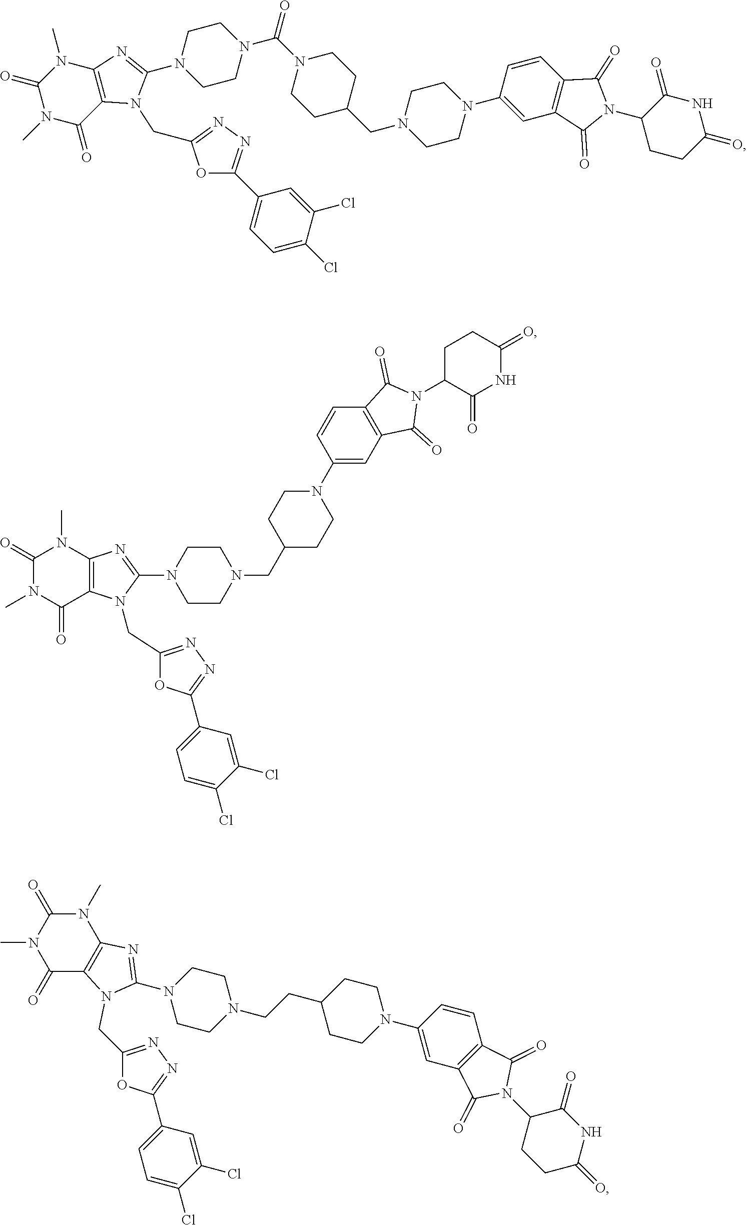 [SMILES: Cn1c(=O)c2c(nc(N3CCN(C(=O)N4CCC(CN5CCN(c6ccc7c(c6)C(=O)N(C6CCC(=O)NC6=O)C7=O)CC5)CC4)CC3)n2Cc2nnc(-c3ccc(Cl)c(Cl)c3)o2)n(C)c1=O ; Cn1c(=O)c2c(nc(N3CCN(CC4CCN(c5ccc6c(c5)C(=O)N(C5CCC(=O)NC5=O)C6=O)CC4)CC3)n2Cc2nnc(-c3ccc(Cl)c(Cl)c3)o2)n(C)c1=O ; Cn1c(=O)c2c(nc(N3CCN(CCC4CCN(c5ccc6c(c5)C(=O)N(C5CCC(=O)NC5=O)C6=O)CC4)CC3)n2Cc2nnc(-c3ccc(Cl)c(Cl)c3)o2)n(C)c1=O] 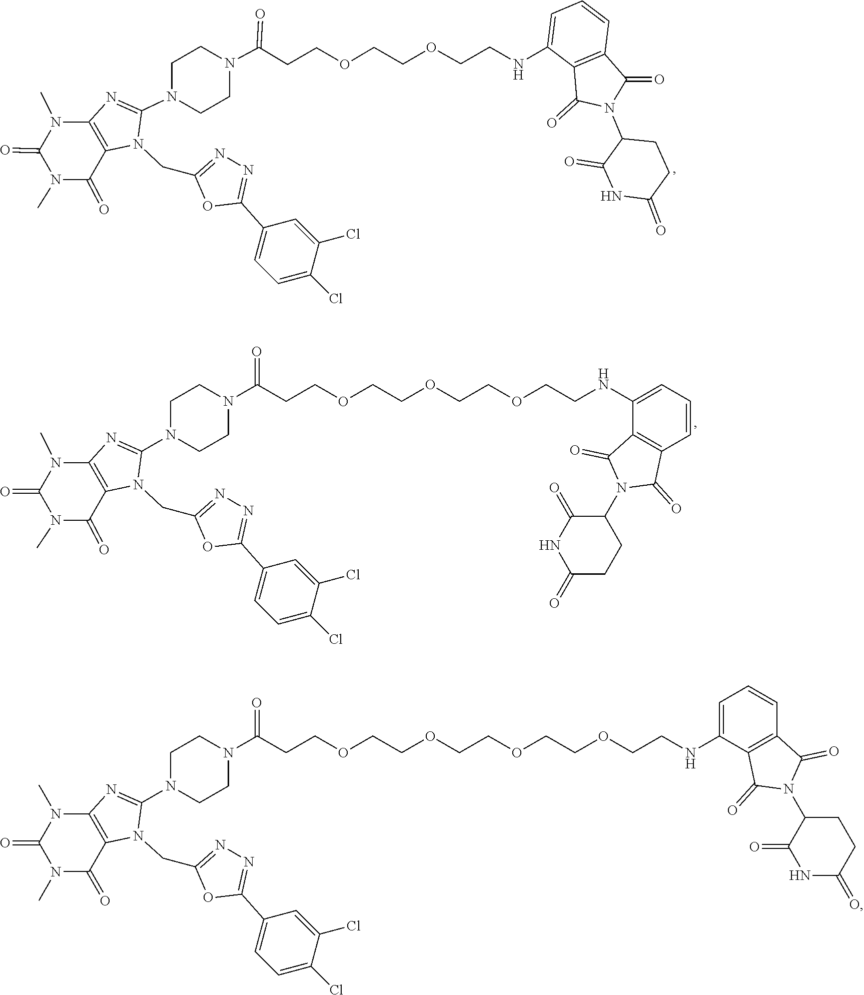 [SMILES: Cn1c(=O)c2c(nc(N3CCN(C(=O)CCOCCOCCNc4cccc5c4C(=O)N(C4CCC(=O)NC4=O)C5=O)CC3)n2Cc2nnc(-c3ccc(Cl)c(Cl)c3)o2)n(C)c1=O ; Cn1c(=O)c2c(nc(N3CCN(C(=O)CCOCCOCCOCCNc4cccc5c4C(=O)N(C4CCC(=O)NC4=O)C5=O)CC3)n2Cc2nnc(-c3ccc(Cl)c(Cl)c3)o2)n(C)c1=O ; Cn1c(=O)c2c(nc(N3CCN(C(=O)CCOCCOCCOCCOCCNc4cccc5c4C(=O)N(C4CCC(=O)NC4=O)C5=O)CC3)n2Cc2nnc(-c3ccc(Cl)c(Cl)c3)o2)n(C)c1=O]
or a pharmaceutically acceptable salt thereof.

17. The compound according to claim 1 which is:

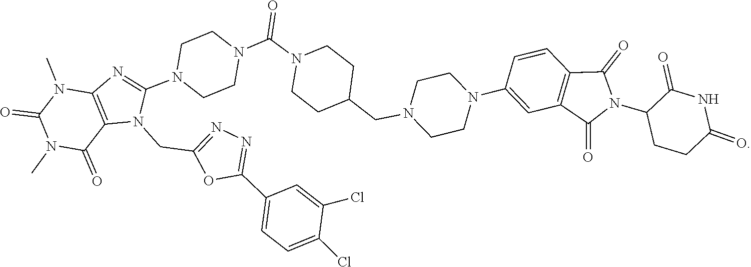 [SMILES: Cn1c(=O)c2c(nc(N3CCN(C(=O)N4CCC(CN5CCN(c6ccc7c(c6)C(=O)N(C6CCC(=O)NC6=O)C7=O)CC5)CC4)CC3)n2Cc2nnc(-c3ccc(Cl)c(Cl)c3)o2)n(C)c1=O]

18. A compound of formula (IB): 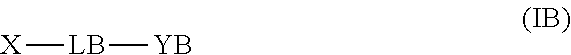
or a pharmaceutically acceptable salt thereof,
wherein X is a moiety capable of binding to NUDT5;
LB is a linker; and
YB is a moiety capable of recruiting cellular destruction machinery, except that YB is not a moiety capable of binding to VHL or CRBN.

19. The compound or pharmaceutically acceptable salt according to claim 18, wherein:
(a) linker LB is a linker of formula L or LA as described herein; and/or
(b) YB is selected from a hydrophobic tag, an autophagy-targeting moiety, a moiety capable of binding to E2 ligase, a moiety capable of binding to E3 ligase other than a moiety capable of binding to VHL or CRBN, and a proteasome recruiter; and/or
(c) X is selected from:

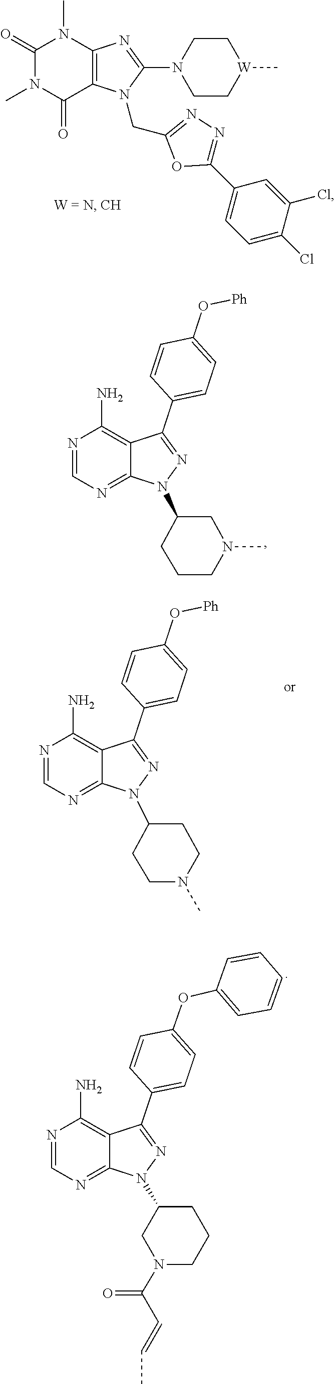

20. A pharmaceutical composition comprising a compound or pharmaceutically acceptable salt according to claim 1 and a pharmaceutically acceptble excipient or carrier.

21. The pharmaceutical composition according to claim 20 further comprising methotrexate and/or 6-thioguanine.

22. A compound or pharmaceutically acceptable salt of claim 1, for use in the treatment of the human or animal body.

23. The compound or composition for use according to claim 22 wherein the compound or composition is for use in a method of treating an inflammatory disorder, autoimmune disorder, cancer or metabolic disorder in a subject.

24-25. (canceled)


SEQUENCE LISTING (15 sequences)

SEQ ID NO: 1 - DNA, 18 bp, synthetic construct
  source 1..18: mol_type = other DNA
tactacaatgaagccaca

SEQ ID NO: 2 - DNA, 19 bp, synthetic construct
  source 1..19: mol_type = other DNA
agactgaccaaatacaaag

SEQ ID NO: 3 - DNA, 20 bp, synthetic construct
  source 1..20: mol_type = other DNA
tcactccacagacctcatgg

SEQ ID NO: 4 - DNA, 20 bp, synthetic construct
  source 1..20: mol_type = other DNA
gaagatgccacggtacaggt

SEQ ID NO: 5 - DNA, 20 bp, synthetic construct
  source 1..20: mol_type = other DNA
atcgtcctcaccaagtctgg

SEQ ID NO: 6 - DNA, 20 bp, synthetic construct
  source 1..20: mol_type = other DNA
gaagatgccacggtacaggt

SEQ ID NO: 7 - DNA, 20 bp, synthetic construct
  source 1..20: mol_type = other DNA
gcggtaccagcgcgactacg

SEQ ID NO: 8 - DNA, 20 bp, synthetic construct
  source 1..20: mol_type = other DNA
ctgcgcgtgaaccgcgccaa

SEQ ID NO: 9 - DNA, 20 bp, synthetic construct
  source 1..20: mol_type = other DNA
tttcaaggaacctaacgcag

SEQ ID NO: 10 - DNA, 20 bp, synthetic construct
  source 1..20: mol_type = other DNA
tttgtgttgtgctcacatcg

SEQ ID NO: 11 - DNA, 20 bp, synthetic construct
  source 1..20: mol_type = other DNA
agcccacccctagtactggg

SEQ ID NO: 12 - DNA, 20 bp, synthetic construct
  source 1..20: mol_type = other DNA
cccaacctctgtcatcacac

SEQ ID NO: 13 - DNA, 20 bp, synthetic construct
  source 1..20: mol_type = other DNA
acaacgtacatggatcctac

SEQ ID NO: 14 - DNA, 20 bp, synthetic construct
  source 1..20: mol_type = other DNA
caaacagtatatcatttcag

SEQ ID NO: 15 - DNA, 20 bp, synthetic construct
  source 1..20: mol_type = other DNA
gtccctgccatcaagcaggg